(19) United States
(12) Patent Application Publication
(10) Pub. No.: US 2022/0106607 A1
(43) Pub. Date: Apr. 7, 2022

(54) GENE FOR PARTHENOGENESIS

(71) Applicant: Keygene N.V., Wageningen (NL)

(72) Inventors: Charles Joseph UNDERWOOD, Wageningen (NL); Diana RIGOLA, Wageningen (NL); Peter Johannes VAN DIJK, Wageningen (NL); Rik Hubertus Martinus OP DEN CAMP, Wageningen (NL); Michael Eric SCHRANZ, Wageningen (NL); Catharina Adriana VIJVERBERG, Wageningen (NL)

(73) Assignee: Keygene N.V., Wageningen (NL)

(21) Appl. No.: 17/535,297

(22) Filed: Nov. 24, 2021

Related U.S. Application Data
(63) Continuation of application No. PCT/EP2020/064991, filed on May 29, 2020.

(30) Foreign Application Priority Data
May 29, 2019 (EP) 19177252.4
Oct. 1, 2019 (EP) 19200872.0
Apr. 17, 2020 (EP) 20170243.8

(51) Int. Cl. C12N 15/82; C07K 14/415

(52) U.S. Cl. CPC C12N 15/8287; C07K 14/415

(57) ABSTRACT

The invention provides the nucleotide sequence and amino acid sequences of the parthenogenesis gene of Taraxacum as well as (functional) homologues, fragments and variants thereof, which provides parthenogenesis as a part of apomixis. Also parthenogenetic plants and methods for making these are provided, as are molecular markers and methods of using these.

### CROSS-REFERENCE TO RELATED APPLICATIONS

The present application is a Continuation of International Patent Application No. PCT/EP2020/064991, filed May 29, 2020, which claims priority to: Europe Patent Application No. 19177252.4 filed May 29, 2019; Europe Patent Application No. 19200872.0 filed Oct. 1, 2019; and Europe Patent Application No. 20170243.8 filed Apr. 4, 2020; the entire contents of all of which are hereby incorporated by reference.

### SEQUENCE LISTING

The instant application contains a Sequence Listing which has been submitted electronically in ASCII format and is hereby incorporated by reference in its entirety. Said ASCII copy, created on Oct. 15, 2021, is named 085342-4100_SequenceListing.txt and is 155,000 bytes in size.

### FIELD OF THE INVENTION

The present invention relates to the field of biotechnology and in particular to plant biotechnology including plant breeding. The invention relates in particular to the identification and uses of genes relating to and useful e.g. in apomixis and haploid induction. The invention in particular relates to the gene that is associated with parthenogenesis, as well as the encoded protein, and fragments of both. The invention further relates to methods for suppressing and/or inducing parthenogenesis in plants and crops, to the use of the gene and/or the protein or their fragments for apomixis in particular in combination with apomeiotic gene(s), or for the production of haploid plants of which the chromosomes can be doubled to produce doubled haploids.

### BACKGROUND OF THE INVENTION

Apomixis (also called agamospermy) is asexual plant reproduction through seeds. Apomixis has been reported in some 400 flowering plant species (Bicknell and Koltunow, 2004). Apomixis in flowering plants occurs in two forms:

(1) gametophytic apomixis, in which the embryo arises from an unreduced, unfertilized egg cell by parthenogenesis;
(2) sporophytic apomixis in which the embryo arises somatically from a sporophytic cell.

Examples of gametophytic apomicts are dandelions (Taraxacum sp.), hawkweeds (Hieracium sp.), Kentucky blue grass (Poa pratensis) and eastern gamagrass (Tripsacum dactyloides). Examples of sporophytic apomixis are citrus (Citrus sp.) and mangosteen (Garcinia mangostana). Gametophytic apomixis involves two developmental processes:

(1) the avoidance of meiotic recombination and reduction (apomeiosis); and
(2) development of the egg cell into an embryo, without fertilization (parthenogenesis).

Apomictically produced seeds are genetically identical to the parental plant. It has been recognized since long that apomixis can be extremely useful in plant breeding (Asker, 1979; Hermsen, 1980; Asker and Jerling, 1990; Vielle-Calzada et al., 1995). The most obvious advantage of the introduction of apomixis into crops is the true breeding of heterotic F1 hybrids. In most crops F1 hybrids are the best performing varieties. However, in sexual crops F1 hybrids have to be produced each generation again by crossing of inbred homozygous parents, because self-fertilization of F1 hybrids causes loss of heterosis by recombination in the genomes of the F2 progeny plants. Producing sexual F1 seeds is a recurrent, complicated and costly process. In contrast, apomictic F1 hybrids would breed true eternally. In other words, genetic fixation of F1 hybrids and production of uniform progeny plants through seed becomes possible.

F1 fixation by apomixis is a special case of the general property of apomixis that any genotype, whatever its genetic complexity, would breed true in one step. This implies that apomixis could be used for immediate fixation of polygenic quantitative traits. It should be noted that most yield traits are polygenic. Apomixis could be used for the stacking (or pyramiding) of multiple traits (for example various resistances, several transgenes, or multiple quantitative trait loci). Without apomixis, in order to fix such a suite of traits, each trait locus must be made homozygous individually and later on combined. As the number of loci involved in a trait increases, the making of these trait loci homozygous by crossing becomes time consuming, logistically challenging and thereby costly. Moreover specific epistatic interactions between alleles are lost by homozygosity. With apomixis it becomes possible to fix this type of non-additive genetic variation. Therefore, apomixis, clonal reproduction through seeds, has the potential to cause of paradigm shift in plant breeding, commercial seed production and agriculture (van Dijk et al. 2016, Van Dijk and Schauer 2016).

Besides instantaneously fixing any genotype, whatever its complexity, there are important additional agricultural uses of apomixis. Sexual interspecific hybrids and autopolyploids often suffer from sterility due to meiotic problems. Since apomixis skips meiosis, with apomixis these problems of interspecific hybrids and autopolyploids can be solved. Since apomixis prevents female hybridization, apomixis coupled with male sterility has been proposed for the containment of transgenes, preventing transgene introgression in wild relatives of transgenic crops (Daniell, 2002). In insect pollinated crops (e.g. Brassica) apomictic seed set would not be limited by insufficient pollinator services. This is becoming more important in the light of the increasing health problems of pollinating bee populations (Varroa mite infections, African killer bees etc.). In tuber propagated crops, like potato, apomixis would maintain the superior genotype clonally, but reduce or even remove the current risk of virus transmission and related cost in clean production, containment and certification. Also the storage costs of apomictic seeds are much less than that of tubers or other vegetatively propagated plant parts. In ornamentals apomixis could replace labour intensive and expensive tissue culture propagation. It is thought that in general apomixis strongly reduces the costs of cultivar development and plant propagation.

Unfortunately apomixis does not occur in any of the major crops. There have been numerous attempts to introduce apomixis in sexual crops. For instance, introgression of apomixis genes, mutation of sexual model species, de novo generation of apomixis by hybridization, and cloning of candidate genes. Introgression of apomixis genes from wild apomicts into crop species through wide crosses have not been successful so far (e.g. apomixis from Tripsacum dactyloides into maize—Savidan, Y., 2001; Morgan et al., 1998; WO97/10704). As to mutating sexual model species, WO2007/066214 describes the use of an apomeiosis mutant called Dyad in Arabidopsis. However, the Dyad is a recessive mutation with very low penetrance. In a crop species this mutation is of limited use. Generation of apomixis de novo by hybridization between two sexual ecotypes has not resulted in agronomical interesting apomicts (US2004/0168216 A1 and US2005/0155111 A1). Cloning of candidate apomixis genes by transposon tagging in maize has been described in US2004/0148667. Orthologs of the elongate gene have been claimed, which are supposed to induce apomixis. However, according to Barrell and Grossniklaus (2005), the elongate gene skips meiosis II and therefore does not maintain the maternal genotype, which makes it much less useful.

It has been described in US2006/0179498 that so called Reverse Breeding would be an alternative for apomixis. However, this is a technically complicated in vitro laboratory procedure, whereas apomixis is an in vivo procedure that is carried out by the plants themselves. Moreover, with reverse breeding, once the parental lines have been reconstructed (doubled gamete homozygotes) crossing still has to be carried out.

Apomixis in natural apomicts generally has a genetic basis (reviewed by Ozias-Akins and Van Dijk, 2007). Therefore an alternative method could be the isolation of apomixis genes from natural apomictic species. However this is not an easy task, because natural apomicts often have a polyploid genome and positional cloning in polyploids is very difficult. Other complicating factors are suppression of recombination in apomixis specific chromosomal regions, repetitive sequences and segregation distortion is crosses.

### SUMMARY OF THE INVENTION

As described herein, there is a need for procedures for inducing apomixis in crops, which are devoid of at least some of the limitations of the present state of the art. Particularly, there is a need for methods for producing apomictic plants and apomictic seeds. There is also a need to provide for genes and proteins involved in the process of apomixis, particularly parthenogenesis, which are suitable for use in introducing apomixis in crops and which can substantially mimic apomictic pathways.

The inventors have now identified and isolated the parthenogenesis locus and gene, the alleles associated with the parthenogenetic phenotype (indicated herein as the parthenogenetic allele or Par allele) and the non-parthenogenetic phenotype (indicated herein as the sexual or non-parthenogenesis allele or par allele), their genetic sequences, i.e. promoter or 5′UTR sequences, coding sequences, 3′UTR sequences and encoded protein sequences. Parthenogenesis can be directly introduced into sexual plants, possibly by random or targeted mutagenesis, by transformation or by somatic hybridization. By genetically modifying the sexual alleles of the parthenogenesis locus of sexual plants, e.g. by mutagenesis, transgenesis or by insertion via introduction of double strand breaks at specific sites and homologous recombination, a Par allele may be introduced and the plant and/or its offspring may become capable of developing an egg cell into an embryo.

### Definitions

As used herein, the term “locus” (plural: loci) means a specific place (or places) or a site on a chromosome where for example a gene or genetic marker is found. For example, the “parthenogenesis locus” refers to the position in the genome where the parthenogenesis gene is located, the allele contributing to the parthenogenetic phenotype i.e. the (parthenogenesis allele or Par allele) and/or its sexual counterpart(s), i.e. the non-parthenogenesis gene(s) (non-parthenogenesis allele(s) or par allele(s)). A gene, allele, protein or nucleic acid being “functional in parthenogenesis” is to be understood herein as contributing to the parthenogenetic phenotype and/or converting the ability to a plant or plant cell to develop an egg cell into an embryo.

As used herein, the term “allele(s)” means any of one or more alternative forms of a gene at a particular locus. In a diploid and/or polyploid cell of an organism, alleles of a given gene are located at a specific location, or locus on a chromosome, wherein one allele is present on each chromosome of the set of homologous chromosomes. A diploid and/or polyploid, or plant species may comprise a large number of different alleles at a particular locus.

The term “dominant allele” as used herein refers the relationship between alleles of one gene in which the effect on phenotype of one allele (i.e. the dominant allele) masks the contribution of a second allele (i.e. the recessive allele) at the same locus. For genes on an autosome (any chromosome other than a sex chromosome), the alleles and their associated traits are autosomal dominant or autosomal recessive. Dominance is a key concept in Mendelian inheritance and classical genetics. For example, a dominant allele may code for a functional protein whereas the recessive allele does not. In an embodiment, the genes and fragments or variants thereof as taught herein refer to dominant alleles of the parthenogenesis gene.

The term “female ovary” (plural: “ovaries”) as used herein refers to an enclosure in which spores are formed. It can be composed of a single cell or can be multicellular. All plants, fungi, and many other lineages form ovaries at some point in their life cycle. Ovaries can produce spores by mitosis or meiosis. Generally, within each ovary, meiosis of a megaspore mother cell produces four haploid megaspores. In gymnosperms and angiosperms, only one of these four megaspores is functional at maturity, and the other three degenerate. The megaspore that pertains divides mitotically and develops into the female gametophyte (megagametophyte), which eventually produces one egg cell.

The term “female gamete” as used herein refers to a cell that fuses under normal (sexual) circumstances with another (“male”) cell during fertilization (conception) in organisms that sexually reproduce. In species that produce two morphologically distinct types of gametes, and in which each individual produces only one type, a female is any individual that produces the larger type of gamete (called an ovule (ovum) or egg cell). In plants, the female ovule is produced by the ovary of the flower. When mature, the haploid ovule produces the female gamete which is then ready for fertilization. The male cell is (mostly haploid) pollen and is produced by the anther.

The term “genetic marker” or “polymorphic marker” refers to a region on the genomic DNA which can be used to “mark” a particular location on the chromosome. If a genetic marker is tightly linked to a gene or is ‘on’ a gene it “marks” the DNA on which the gene is found and can therefore be used in a (molecular) marker assay to select for or against the presence of the gene, e.g. in marker assisted breeding/selection (MAS) methods. Examples of genetic markers are AFLP (amplified fragment length polymorphism, EP534858), microsatellite, RFLP (restriction fragment length polymorphism), STS (sequence tagged site), SNP (Single Nucleotide Polymorphism), SFP (Single Feature Polymorphism; see Borevitz et al., 2003), SCAR (sequence characterized amplified region), CAPS markers (cleaved amplified polymorphic sequence) and the like. The further away the marker is from the gene, the more likely it is that recombination (crossing over) takes place between the marker and the gene, whereby the linkage (and co-segregation of marker and gene) is lost. The distance between genetic loci is measured in terms of recombination frequencies and is given in cM (centiMorgans; 1 cM is a meiotic recombination frequency between two markers of 1%). As genome sizes vary greatly between species, the actual physical distance represented by 1 cM (i.e. the kilobases, kb, between two markers) also varies greatly between species.

It is understood that, when referring to “linked” markers herein, this also encompasses markers “on” the gene itself.

“MAS” refers to “marker assisted selection”, whereby plants are screened for the presence and/or absence of one or more genetic and/or phenotypic markers in order to accelerate the transfer of the DNA region comprising the marker (and optionally lacking flanking regions) into an (elite) breeding line.

A “molecular marker assay” (or test) refers to a (DNA based) assay that indicates (directly or indirectly) the presence or absence of an allele e.g. a Par or par allele in a plant or plant part. Preferably it allows one to determine whether a particular allele is homozygous or heterozygous at the parthenogenesis locus in any individual plant. For example, in one embodiment a nucleic acid linked to the parthenogenesis locus is amplified using PCR primers, the amplification product is digested enzymatically and, based on the electrophoretically resolved patterns of the amplification product, one can determine which allele(s) is/are present in any individual plant and the zygosity of the allele at the parthenogenesis locus (i.e. the genotype at each locus). Examples are SCAR markers (sequence characterized amplified region), CAPS markers (cleaved amplified polymorphic sequence) and similar marker assays.

As used herein, the term “heterozygous” means a genetic condition existing when two different alleles reside at a specific locus, but are positioned individually on corresponding sets of homologous chromosomes in the cell. Conversely, as used herein, the term “homozygous” means a genetic condition existing when two (or more in case of polyploidy) identical alleles reside at a specific locus, but are positioned individually on corresponding sets of homologous chromosomes in the cell.

A “variety” is used herein in conformity with the UPOV convention and refers to a plant grouping within a single botanical taxon of the lowest known rank, which grouping can be defined by the expression of the characteristics and can be distinguished from any other plant grouping by the expression of at least one of the said characteristics and is considered as a unit with regard to its suitability for being propagated unchanged (stable).

The terms “protein” or “polypeptide” are used interchangeably and refer to molecules consisting of a chain of amino acids, without reference to a specific mode of action, size, 3 dimensional structure or origin. A “fragment” or “portion” of a protein may thus still be referred to as a “protein”. An “isolated protein” is used to refer to a protein which is no longer in its natural environment, for example in vitro or in a recombinant bacterial or plant host cell.

The term “gene” means a DNA sequence comprising a region (transcribed region), which is transcribed into an RNA molecule (e.g. an pre-mRNA which is processed to an mRNA) in a cell, operably linked to suitable regulatory regions (e.g. a promoter). A gene may thus comprise several operably linked sequences, such as a promoter, a 5′ leader sequence comprising e.g. sequences involved in translation initiation, a (protein) coding region (cDNA or genomic DNA) and a 3′ non-translated sequence comprising e.g. transcription termination sites.

A “chimeric gene” (or recombinant gene) refers to any gene, which is not normally found in nature in a species, in particular a gene in which one or more parts of the nucleotide sequence are present that are not associated with each other in nature. For example the promoter is not associated in nature with part or all of the transcribed region or with another regulatory region. The term “chimeric gene” is understood to include expression constructs in which a promoter or transcription regulatory sequence is operably linked to one or more coding sequences or to an antisense (reverse complement of the sense strand) or inverted repeat sequence (sense and antisense, whereby the RNA transcript forms double stranded RNA upon transcription).

A “3′UTR” or “3′ non-translated sequence” (also often referred to as 3′ untranslated region, or 3′ end) refers to the nucleotide sequence found downstream of the coding sequence of a gene, which comprises for example a transcription termination site and (in most, but not all eukaryotic mRNAs) a polyadenylation signal (such as e.g. AAUAAA or variants thereof). After termination of transcription, the mRNA transcript may be cleaved downstream of the polyadenylation signal and a poly(A) tail may be added, which is involved in the transport of the mRNA to the cytoplasm (where translation takes place).

A “5′UTR” or “leader sequence” or “5′ untranslated region” is a region of the mRNA transcript, and the corresponding DNA, between the +1 position where mRNA transcription begins and the translation start codon of the coding region (usually AUG on the mRNA or ATG on the DNA). The 5′UTR usually contains sites important for translation, mRNA stability and/or turnover, and other regulatory elements.

“Expression of a gene” refers to the process wherein a DNA region, which is operably linked to appropriate regulatory regions, particularly a promoter, is transcribed into an RNA, which is biologically active, i.e. which is capable of being translated into a biologically active protein or peptide (or active peptide fragment) or which is active itself (e.g. in posttranscriptional gene silencing or RNAi). An active protein in certain embodiments refers to a protein being constitutively active. The coding sequence is preferably in sense-orientation and encodes a desired, biologically active protein or peptide, or an active peptide fragment. In gene silencing approaches, the DNA sequence is preferably present in the form of an antisense DNA or an inverted repeat DNA, comprising a short sequence of the target gene in antisense or in sense and antisense orientation.

A “transcription regulatory sequence” is herein defined as a nucleotide sequence that is capable of regulating the rate of transcription of a (coding) sequence operably linked to the transcription regulatory sequence. A transcription regulatory sequence as herein defined will thus comprise all of the sequence elements necessary for initiation of transcription (promoter elements), for maintaining and for regulating transcription, including e.g. attenuators or enhancers. Although mostly the upstream (5′) transcription regulatory sequences of a coding sequence are referred to, regulatory sequences found downstream (3′) of a coding sequence are also encompassed by this definition.

As used herein, the term “promoter” refers to a nucleic acid fragment that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. Optionally the term “promoter” includes herein also the 5′UTR region (e.g. the promoter may herein include one or more parts upstream (5′) of the translation initiation codon of a gene, as this region may have a role in regulating transcription and/or translation. A “constitutive” promoter is a promoter that is active in most tissues under most physiological and developmental conditions. An “inducible” promoter is a promoter that is physiologically (e.g. by external application of certain compounds) or developmentally regulated. A “tissue specific” promoter is only active in specific types of tissues or cells. A “promoter active in plants or plant cells” refers to the general capability of the promoter to drive transcription within a plant or plant cell. It does not make any implications about the spatiotemporal activity of the promoter.

As used herein, the term “operably linked” refers to a linkage of polynucleotide elements in a functional relationship. A nucleic acid is “operably linked” when it is placed into a functional relationship with another nucleotide sequence. For instance, a promoter, or rather a transcription regulatory sequence, is operably linked to a coding sequence if it affects the transcription of the coding sequence. Operably linked means that the DNA sequences being linked are typically contiguous and, where necessary to join two protein encoding regions, contiguous and in reading frame so as to produce a “chimeric protein”. A “chimeric protein” or “hybrid protein” is a protein composed of various protein “domains” (or motifs) which is not found as such in nature but which a joined to form a functional protein, which displays the functionality of the joined domains. A chimeric protein may also be a fusion protein of two or more proteins occurring in nature. The term “domain” as used herein means any part(s) or domain(s) of the protein with a specific structure or function that can be transferred to another protein for providing a new hybrid protein with at least the functional characteristic of the domain.

The terms “target peptide” refers to amino acid sequences which target a protein, or protein fragment, to intracellular organelles such as plastids, preferably chloroplasts, mitochondria, or to the extracellular space or apoplast (secretion signal peptide). A nucleotide sequence encoding a target peptide may be fused (in frame) to the nucleotide sequence encoding the amino terminal end (N-terminal end) of the protein or protein fragment, or may be used to replace a native targeting peptide.

A “nucleic acid construct” or “vector” is herein understood to mean a man-made nucleic acid molecule resulting from the use of recombinant DNA technology and which is used to deliver exogenous DNA into a host cell. The vector backbone may for example be a binary or superbinary vector (see e.g. U.S. Pat. No. 5,591,616, US 2002138879 and WO95/06722), a co-integrate vector or a T-DNA vector, as known in the art and as described elsewhere herein, into which a gene or chimeric gene is integrated or, if a suitable transcription regulatory sequence is already present, only a desired nucleotide sequence (e.g. a coding sequence, an antisense or an inverted repeat sequence) is integrated downstream of the transcription regulatory sequence. Vectors usually comprise further genetic elements to facilitate their use in molecular cloning, such as e.g. selectable markers, multiple cloning sites and the like.

A “recombinant host cell” or “transformed cell” or “transgenic cell” are terms referring to a new individual cell (or organism) arising as a result of at least one nucleic acid molecule, especially comprising a gene or chimeric gene encoding a desired protein or a nucleotide sequence which upon transcription yields an antisense RNA or an inverted repeat RNA (or hairpin RNA) for silencing of a target gene/gene family, having been introduced into said cell. An “isolated nucleic acid” is used to refer to a nucleic acid which is no longer in its natural environment, for example in vitro or in a recombinant bacterial or plant host cell.

A host cell” is the original cell to be transformed with a transgene to become a recombinant host cell. The host cell is preferably a plant cell or a bacterial cell. The recombinant host cell may contain the nucleic acid construct as an extra-chromosomally (episomal) replicating molecule, or more preferably, comprises the gene or chimeric gene integrated in the nuclear or plastid genome of the host cell.

A “recombinant plant” or “recombinant plant part” or “transgenic plant” is a plant or plant part (seed or fruit or leaves, for example) which comprises a recombinant gene or chimeric gene, even though the gene may not be expressed, or not be expressed in all cells.

An “elite event” is a recombinant plant which has been selected to comprise the recombinant gene at a position in the genome which results in good phenotypic and/or agronomic characteristics of the plant. The flanking DNA of the integration site can be sequenced to characterize the integration site and distinguish the event from other transgenic plants comprising the same recombinant gene at other locations in the genome.

The term “selectable marker” is a term familiar to one of ordinary skill in the art and is used herein to describe any genetic entity which, when expressed, can be used to select for a cell or cells containing the selectable marker. Selectable marker gene products confer for example antibiotic resistance, or more preferably, herbicide resistance or another selectable trait such as a phenotypic trait (e.g. a change in pigmentation) or a nutritional requirement. The term “reporter” is mainly used to refer to visible markers, such as green fluorescent protein (GFP), eGFP, luciferase, GUS and the like.

The term “orthologue” of a gene or protein refers herein to the homologous gene or protein found in another species, which has the same function as the gene or protein, but (usually) diverged in sequence from the time point on when the species harboring the genes diverged (i.e. the genes evolved from a common ancestor by speciation). Orthologues of the Taxaracum parthenogenesis gene may thus be identified in other plant species based on both sequence comparisons (e.g. based on percentages sequence identity over the entire sequence or over specific domains) and functional analysis.

The terms “homologous” and “heterologous” refer to the relationship between a nucleic acid or amino acid sequence and its host cell or organism, especially in the context of transgenic organisms. A homologous sequence is thus naturally found in the host species (e.g. a lettuce plant transformed with a lettuce gene), while a heterologous sequence is not naturally found in the host cell (e.g. a lettuce plant transformed with a sequence from potato plants). Depending on the context, the term “homologue” or “homologous” may alternatively refer to sequences which are descendent from a common ancestral sequence (e.g. they may be orthologues).

“Stringent hybridization conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tm) for the specific sequences at a defined ionic strength and pH. The Tm is the temperature (under defined ionic strength and pH) at which 50% of the target sequence hybridizes to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridizations (Northern blots using a probe of e.g. 100 nt) are for example those which include at least one wash in 0.2×SSC at 63° C. for 20 min, or equivalent conditions. Stringent conditions for DNA-DNA hybridization (Southern blots using a probe of e.g. 100 nt) are for example those which include at least one wash (usually 2) in 0.2×SSC at a temperature of at least 50° C., usually about 55° C., for 20 min, or equivalent conditions. See also Sambrook et al. (1989) and Sambrook and Russell (2001).

“High stringency” conditions can be provided, for example, by hybridization at 65° C. in an aqueous solution containing 6×SSC (20×SSC contains 3.0 M NaCl, 0.3 M Na-citrate, pH 7.0), 5×Denhardt's (100×Denhardt's contains 2% Ficoll, 2% Polyvinyl pyrollidone, 2% Bovine Serum Albumin), 0.5% sodium dodecyl sulphate (SDS), and 20 μg/ml denaturated carrier DNA (single-stranded fish sperm DNA, with an average length of 120-3000 nucleotides) as non-specific competitor. Following hybridization, high stringency washing may be done in several steps, with a final wash (about 30 min) at the hybridization temperature in 0.2-0.1×SSC, 0.1% SDS.

“Moderate stringency” refers to conditions equivalent to hybridization in the above described solution but at about 60-62° C. In that case the final wash is performed at the hybridization temperature in 1×SSC, 0.1% SDS.

“Low stringency” refers to conditions equivalent to hybridization in the above described solution at about 50-52° C. In that case, the final wash is performed at the hybridization temperature in 2×SSC, 0.1% SDS. See also Sambrook et al. (1989) and Sambrook and Russell (2001).

“Sequence identity” and “sequence similarity” can be determined by alignment of two peptide or two nucleotide sequences using global or local alignment algorithms, depending on the length of the two sequences. Sequences of similar lengths are preferably aligned using a global alignment algorithms (e.g. Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined herein). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). When sequences have a substantially different overall lengths, local alignments, such as those using the Smith Waterman algorithm, are preferred.

Alternatively percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to oxidoreductase nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

The term “sexual plant reproduction” as used herein refers to a developmental pathway where a (e.g. diploid) somatic cell referred to as the “megaspore mother cell” undergoes meiosis to produce four reduced megaspores. One of these megaspores divides mitotically to form the megagametophyte (also known as the embryo sac), which contains a reduced egg cell (i.e. cell having a reduced number of chromosomes compared to the mother) and two reduced polar nuclei. Fertilization of the egg cell by one sperm cell of the pollen grain generates a (e.g. diploid) embryo, while fertilization of the two polar nuclei by the second sperm cell generates the (e.g. triploid) endosperm (process referred to as double fertilization).

The term “megaspore mother cell” or “megasporocyte” as used herein refers to a cell that produces megaspores by reduction, usually meiosis, to create four haploid megaspores which will develop into female gametophytes. In angiosperms (also known as flowering plants), the megaspore mother cell produces a megaspore that develops into a megagametophyte through two distinct processes including megasporogenesis (formation of the megaspore in the nucellus, or megasporangium), and megagametogenesis (development of the megaspore into the megagametophyte).

The term “asexual plant reproduction” as used herein is a process by which plant reproduction is achieved without fertilization and without the fusion of gametes. Asexual reproduction produces new individuals, genetically identical to the parent plants and to each other, except when mutations or somatic recombinations occur. Plants have two main types of asexual reproduction including vegetative reproduction (i.e. involves budding, tillering, etc of a vegetative piece of the original plant) and apomixis.

The term “apomixis” as used herein refers to the formation of seeds by asexual processes. One form of apomixis is characterized by: 1) apomeiosis, which refers to the formation of unreduced embryo sacs in the ovary, and 2) parthenogenesis, which refers to the development of the unreduced egg into an embryo. A few hundred wild plant species feature apomictic reproduction and propagate asexually. Apomeiosis is a process that results into the production of unreduced egg cells, with the same chromosome number and identical or highly similar genotype as the somatic tissue of the mother plant. The unreduced egg cells can be derived from an unreduced megaspore (diplospory) or from a somatic initial cell (apospory). In the case of diplospory, megasporogenesis is replaced by a mitotic division or by a modified meiosis. The modified meiosis is preferably of the first division restitution type, without recombination. Alternatively the modified meiosis can be of the second division restitution type. In a preferred embodiment, apomeiosis is of the diplosporous type affecting the first meiotic division. Apomixis is known to occur in different forms including at least two forms known as gametophytic apomixis and sporophytic apomixis (also referred to as adventive embryony). Examples of plants where gametophytic apomixis occurs include dandelion (Taraxacum sp.), hawkweed (Hieracium sp.), Kentucky blue grass (Poa pratensis), eastern gamagrass (Tripsacum dactyloides) and others. Examples of plants where sporophytic apomixis occurs include citrus (Citrus sp.) mangosteen (Garcinia mangostana) and others.

The term “diplospory” as used herein refers to a situation where an unreduced embryo sac is derived from the megaspore mother cell either directly by mitotic division or by aborted meiotic events. Three major types of diplospory have been reported, named after the plants in which they occur, and they are the Taraxacum, Ixeris and Antennaria types. In the Taraxacum type, the meiotic prophase is initiated but then the process is aborted resulting in two unreduced dyads one of which gives rise to the embryo sac by mitotic division. In the Ixeris type, two further mitotic divisions of the nuclei to give rise to an eight-nucleate embryo sac follow equational division following meiotic prophase. The Taraxacum and Ixeris types are known as meiotic diplospory because they involve modifications of meiosis. By contrast, in the Antennaria type, referred to as mitotic diplospory, the megaspore mother cell does not initiate meiosis and directly divides three times to produce the unreduced embryo sac. In gametophytic apomixis by diplospory, an unreduced gametophyte is produced from an unreduced megaspore. This unreduced megaspore results from either a mitotic-like division (mitotic displory) or a modified meiosis (meiotic displory). In both gametophytic apomixis by apospory and gametophytic apomixis by diplospory, the unreduced egg cell develops parthenogenetically into an embryo. Apomixis in Taraxacum is of the diplosporous type, which means that the first female reduction division (meiosis I) is skipped, resulting in two unreduced megaspores with the same genotypes as the mother plant. One of these megaspores degenerates and the other surviving unreduced megaspore gives rise to the unreduced megagametophyte (or embryo sac), containing an unreduced egg cell. This unreduced egg cell develops without fertilization into an embryo with the same genotype as the mother plant. The seeds resulting from the process of gametophytic apomixis are referred to as apomictic seeds.

The term “diplospory function” refers to the capability to induce diplospory in a plant, preferably in the female ovary, preferably in a megaspore mother cell and/or in a female gamete. Thus a plant in which diplospory function is introduced, is capable of performing the diplospory process, i.e. producing unreduced gametes via a meiosis I restitution.

The term “diplospory as part of gametophytic apomixis” refers to the diplospory component of the process of apomixis, i.e. the role that diplospory plays in the formation of seeds by asexual processes. In particular, next to diplospory function, parthenogenesis function is required as well in establishing the process of apomixis. Thus, a combination of diplospory and parthenogenesis functions may result in apomixis.

The term “diplosporous plant” as used herein refers to a plant, which undergoes gametophytic apomixis through diplospory or a plant that has been induced (e.g. by genetic modifications) to undergo gametophytic apomixis through diplospory. In both cases, diplosporous plants produce apomictic seeds when combined with a parthenogenesis factor.

The term “apomictic seeds” as used herein refers to seeds, which are obtained from apomictic plant species or by plants or crops induced to undergo apomixis, particularly gametophytic apomixis through diplospory. Apomictic seeds are characterised in that they are a clone and genetically identical to the parent plant and germinate plants that are capable of true breeding. In the present invention, the “apomictic seeds” also refers to “clonal apomictic seeds”.

The term “apomictic plant(s)” as used herein, refers to a plant that reproduce itself asexually, without fertilization. An apomictic plant may be a sexual plant that has been modified to become apomictic, e.g. a sexual plant, which has for instance been genetically modified with one or more of the parthenogenesis genes as taught herein so as to obtain an apomictic plant, or a plant that is the progeny of an apomictic plant. In that case, apomictically produced offspring are genetically identical to the parent plant.

A “clone” of a cell, plant, plant part or seed is characterized in that they are genetically identical to their siblings as well as to the parent plant from which they are derived. Genomic DNA sequences of individual clones are nearly identical, however, mutations may cause minor differences.

The term “true breeding” or “true breeding organism” (also known as pure-bred organism) as used herein refers to an organism that always passes down a certain phenotypic trait unchanged or nearly unchanged to its offspring. An organism is referred to as true breeding for each trait to which this applies, and the term “true breeding” is also used to describe individual genetic traits.

The term “F1 hybrid” (or filial 1 hybrid) as used herein refers to the first filial generation of offspring of distinctly different parental types. The parental types may or may not be inbred lines. F1 hybrids are used in genetics, and in selective breeding, where it may appear as F1 crossbreed. The offspring of distinctly different parental types produce a new, uniform phenotype with a combination of characteristics from the parents. F1 hybrids are associated with distinct advantages such as heterosis, and thus are highly desired in agricultural practice. In an embodiment of the invention, the methods, genes, proteins, variants or fragments thereof as taught herein can be used to fix the genotype of F1 hybrids, regardless of its genetic complexity, and allows production of organisms that can breed true in one step.

The term “pollination” or “pollinating” as used herein refers to the process by which pollen is transferred from the anther (male part) to the stigma (female part) of the plant, thereby enabling fertilization and reproduction. It is unique to the angiosperms, the flower-bearing plants. Each pollen grain is a male haploid gametophyte, adapted to being transported to the female gametophyte, where it can effect fertilization by producing the male gamete (or gametes), in the process of double fertilization. A successful angiosperm pollen grain (gametophyte) containing the male gametes is transported to the stigma, where it germinates and its pollen tube grows down the style to the ovary. Its two gametes travel down the tube to where the gametophyte(s) containing the female gametes are held within the carpel. One nucleus fuses with the polar bodies to produce the endosperm tissues, and the other with the ovule to produce the embryo.

The term “parthenogenesis” as used herein refers to a form of asexual reproduction in which growth and development of embryos occur without fertilization. The genes and proteins of the invention can, in combination with a diplosporous factor, for instance a gene or chemical factor, produce apomictic offspring.

The term “pyramiding or stacking gene” as used herein, refers to the process of combining related or unrelated genes from different parental line into one plant, which underlie desirable or favourable traits (e.g. disease resistance traits, colour, drought resistance, pest resistance, etc.). Pyramiding or stacking gene can be performed using traditional breeding methods or can be accelerated by using molecular markers to identify and keep plants that contain the desired allele combination and discard those that do not have the desired allele combination. In an embodiment of the present invention, the parthenogenesis genes as taught herein may be advantageously used in gene pyramiding or stacking program to produce apomictic plants or to introduce apomixis in sexual crops.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”. It is further understood that, when referring to “sequences” herein, generally the actual physical molecules with a certain sequence of subunits (e.g. amino acids) are referred to.

As used herein, the term “plant” includes plant cells, plant tissues or organs, plant protoplasts, plant cell tissue cultures from which plants can be regenerated, plant calli, plant cell clumps, and plant cells that are intact in plants, or parts of plants, such as embryos, pollen, ovules, fruit, flowers, leaves (e.g. harvested lettuce crops), seeds, roots, root tips and the like.

### DETAILED DESCRIPTION OF THE INVENTION

### Nucleotide Sequences of the Invention

The present inventors for the first time identified the gene, coding sequence, promoter, 3′UTR and protein responsible for parthenogenesis. Said genetic sequence, promoter sequence, coding sequence and 3′UTR sequence are located on the Par allele. The inventors also identified the genetic sequences, promoter sequences, coding sequences, and 3′UTR sequences located on the sexual counterparts of the Par allele, i.e. on the par alleles. As sexual counterparts of the dominant allele that causes parthenogenesis, these par alleles are indicated also herein as being associated with parthenogenesis, albeit that their presence does not contribute to the parthenogenetic phenotype, as the presence of a par allele may be indicative for the sexual phenotype, i.e. the non-parthenogenic phenotype. As the Par allele may be a dominant allele, confirmation of the sexual phenotype may require the assessment of all alleles of the Par locus as par alleles and/or the require the assessment of the absence of a Par allele. In other words “associated with” is herein to be understood as indicative for the parthenogenic or the non-parthenogenic phenotype, and optionally for being functional in parthenogenesis. Modification of a par allele, for instance by modifying one or more expression regulatory sequences of the par allele such as the promoter sequence that results in altered expression of the encoded protein, may confer the par allele to a Par allele capable of inducing a parthenogenetic phenotype.

Both the Par and par alleles comprise genes with coding sequences that encode a protein denominated herein as the “PAR protein”, which comprises a zinc finger C2H2-type domain (IPR13087), preferably a zinc finger K2-2-like domain having the consensus sequence C.{2}C.{7}[K/R]A.{2}GH [R/N].H, which can also be annotated as: CXXCXXXXXXX[K/R]AXXGHX[R/N]XH (SEQ ID NO: 37), wherein X may be any naturally occurring amino acid, wherein [K/R] indicates that the amino acid on position 12 is lysine or arginine, and wherein R/N] indicates that the amino acid on position 19 is arginine or asparagine (see Englbrecht et al., 2004). In addition to the zinc finger C2H2-type domain, preferably a zinc finger K2-2-like domain as defined herein, the protein comprises an EAR motif having the consensus amino acid sequence DLNXXP (SEQ ID NO: 58) or DLNXP (SEQ ID NO: 59), wherein X may by any naturally occurring amino acid (see Kagale et al., 2010). Preferably, the protein is at most 400 amino acids, wherein said protein comprises one or two EAR motifs as indicated herein and a zinc finger K2-2-like domain as defined herein. Preferably, the protein is at most 400 amino acids, wherein said protein comprises only one or two EAR motifs as indicated herein and only one zinc finger K2-2-like domain as defined herein, i.e. no further EAR motifs as defined herein and no further zinc finger K2-2-like domains as defined herein. In addition to the features of the maximum size of 400 amino acids, the only one or two EAR motifs as indicated herein and a single zinc finger K2-2-like domain as defined, the PAR protein may comprise only one further zinc finger domain having the zinc finger consensus sequence of C.{2}C.{12}H.{3}H, which can also be annotated as: CXXCXXXXXXXXXXXXHXXH (SEQ ID NO: 38), but more preferably comprises no further zinc finger domains having the zinc finger consensus sequence of C.{2}C.{12}H.{3}H (SEQ ID NO: 38).

The invention therefore provides for a nucleic acid that is associated with parthenogenesis in plants, wherein said nucleic acid comprises a nucleotide sequence encoding the PAR protein as defined herein. The invention also provides for the promoter sequence and 3′UTR operably linked to the nucleotide sequence encoding said PAR protein. Taraxacum officinale comprises one dominant Par allele capable of inducing parthenogenesis and two sexual counter parts, i.e. par allele-1 and par allele-2, which encode PAR proteins having the respectively amino acid sequence of SEQ ID NO: 1, 6 or 11. The Par allele comprises a gene having the nucleotide sequence of SEQ ID NO: 5, par allele-1 comprises a par gene having the nucleotide sequence of SEQ ID NO: 10, and par allele-2 comprises a par gene having the nucleotide sequence of SEQ ID NO: 15. The Par gene comprises a promoter sequence having SEQ ID NO: 2, a coding sequence having SEQ ID NO: 3 and a 3′UTRs having SEQ ID NO: 4. The par gene-1 comprises promoter sequence having SEQ ID NO: 7, a coding sequence having SEQ ID NO: 8 and a 3′UTRs having SEQ ID NO: 9. The par gene-2 comprises promoter sequence having SEQ ID NO: 12, a coding sequence having SEQ ID NO: 13 and a 3′UTRs having SEQ ID NO: 14. The invention therefore provides for a nucleic acid that is associated with parthenogenesis in plants, wherein said nucleic acid comprises at least one of:

a) a gene that encodes a protein having an amino acid sequence of SEQ ID NO: 1, 6 or 11;

b) a promoter having the nucleotide sequence of SEQ ID NO: 2, 7 or 12;

c) a coding sequence having the nucleotide sequence of SEQ ID NO: 3, 8 or 13;

d) a 3′UTR having the nucleotide sequence of SEQ ID NO: 4, 9 or 14;

e) a gene having the nucleotide sequence of SEQ ID NO: 5, 10 or 15;

f) a variant of any one of a)-e); and

g) a fragment of any one of a)-f).

Table 1 provides an overview of all SEQ ID NOs used herein.
Preferably said nucleic acid is functional in parthenogenesis.

In one embodiment, the nucleic acid of the invention comprises or consist of at least one of:

a) a gene that encodes a protein having an amino acid sequence of SEQ ID NO: 1;

b) a promoter having the nucleotide sequence of SEQ ID NO: 2;

c) a coding sequence having the nucleotide sequence of SEQ ID NO: 3;

d) a 3′UTR having the nucleotide sequence of SEQ ID NO: 4;

e) a gene having the nucleotide sequence of SEQ ID NO: 5;

f) a variant of any one of a)-e); and

g) a fragment of any one of a)-f).

Preferably, the nucleic acid of this embodiment and/or a product derived therefrom, such as its RNA transcript or encoded protein, is indicative for the parthenogenesis, e.g. a plant comprising said nucleic acid indicates said plant to show parthenogenesis, meaning that it has the ability to develop an embryo from a reduced or unreduced egg cell. Preferably said nucleic acid and/or a product derived therefrom, such as its RNA transcript or encoded protein, is functional in parthenogenesis, even more preferably induces or is capable of inducing parthenogenesis, preferably when present in a plant or plant cell.

In another embodiment, the nucleic acid of the invention comprises or consists of at least one of:

a) a gene that encodes a protein having an amino acid sequence of SEQ ID NO: 6 or 11;

b) a promoter having the nucleotide sequence of SEQ ID NO: 7 or 12;

c) a coding sequence having the nucleotide sequence of SEQ ID NO: 8 or 13;

d) a 3′UTR having the nucleotide sequence of SEQ ID NO: 9 or 14;

e) a gene having the nucleotide sequence of SEQ ID NO: 10 or 15;

f) a variant of any one of a)-e); and

g) a fragment of any one of a)-f).

Preferably, the said nucleic acid of this embodiment and/or a product derived therefrom, such as its RNA transcript or encoded protein, does not induce or is not capable of inducing parthenogenesis, preferably when present in a plant or plant cell in a homozygous state. In other words, the presence of the nucleic acid of this embodiment may be indicative for the non-parthenogenesis phenotype or sexual phenotype, e.g. a plant comprising said nucleic acid indicates said plant to be of the sexual phenotype, i.e. not capable of developing an embryo from an egg cell.

The Par allele may be a dominant allele. In case the Par allele is dominant, in order to confirm that a plant is of the non-parthenogenetic phenotype, all alleles of the Par locus in said plant require to be assessed as par alleles, and the presence of a single Par allele is sufficient to indicate the plant as capable of parthenogenesis.

The nucleic acid of the invention may be used for screening and/or genotyping. Optionally, functionality in parthenogenesis of a putative nucleic acid or gene and/or its derived product, or the capability of a putative nucleic acid and/or its derived product to induce parthenogenesis, may be assessed by reducing expression, by silencing or by knocking out said nucleic acid or gene in a parthenogenetic plant, e.g. by introducing an early stop in the coding sequence of said gene. The subsequent loss of parthenogenetic phenotype means that the putative nucleic acid and/or its derived product is capable of inducing parthenogenesis. Capability to induce parthenogenesis may also be assessed by complementation of a loss-of-function apomictic plant with the putative nucleic acid and/or its derived product (mRNA or protein). Such loss-of-function apomictic plant may be a Taraxacum officinale isolate A68 that has been modified to lose the apomictic phenotype by reducing expression of functional Par allele (e.g. by deletion or knocking out). Such loss-of-function apomictic plant may be a Taraxacum officinale isolate A68 that comprises a Par allele wherein SEQ ID NO: 23 as defined herein has been modified to any one of SEQ ID NO: 24-27 (see Table 2). Such loss of function apomictic plant of Taraxacum officinale isolate A68 may be obtained by targeted genome editing using a CRISPR-Cas9/guide RNA complex, wherein said guide RNA (also indicated herein as gRNA) comprises the target specific sequence of SEQ ID NO: 19, as exemplified herein. Deletion of the Par allele of Taraxacum officinale isolate A68 results in loss-of-parthenogenesis and therefore in loss-of-apomixis. In case said putative nucleic acid, or its derived product, has the capability to induce parthenogenesis, the apomictic phenotype will be restored (or rescued) upon introduction of said nucleic acid or derived product in said isolate, e.g. by transfecting said isolate with a vector comprising said nucleic acid and/or encoding said product. Such vector preferably comprises sequences suitable for driving expression of the encoded product in the isolate. For instance a putative nucleic acid encoding possibly a PAR protein of the invention may be operably linked within said vector to the promoter defined herein by SEQ ID NO: 2 and optionally to 3′UTR defined herein by SEQ ID NO: 4. For Taraxacum officinale isolate A68, high seed set in the absence of cross pollination is a clear indication for apomixis. Selfing in this isolate can be excluded as an alternative explanation, because due to a unbalanced triploid male and female meiosis, sexually produced egg cells and pollen grains will have a very low fertility.

Preferably the variant nucleic acid as defined herein is a homologue or orthologue of gene, promoter, coding sequence and/or 3′UTR of the Par or par alleles of Taraxacum officinale isolate A68 as defined herein. Preferably said variant nucleic acid and/or a product derived therefrom, such as its RNA transcript or encoded protein, is associated with parthenogenesis as defined herein and optionally induces or is capable of inducing parthenogenesis, preferably when present in a plant or plant cell. The variant preferably encodes for, or is operably linked to a sequence encoding, a PAR protein as defined herein. Orthologues of the Par and par genes as identified in Taraxacum officinale isolate A68 in other plant species can be identified based on the characteristics of the PAR protein as defined herein. Such gene may encode for, but is not limited to, any one of the PAR proteins selected from the group consisting of: PAR protein from Ananas comosus (e.g. UniProtKB: A0A199URK4), PAR protein from Apostasia shenzhenica (e.g. UniProtKB: A0A2I0AZW3), PAR protein from Arabidopsis thaliana (e.g. UniProtKB: Q8GXP9, A0A178V2S4, O81793, A0A178V1Q3, A0MFC1, O81801), PAR protein from Arabidopsis lyrata subsp. Lyrata (e.g. UniProtKB: D7MC52 or D7MCE8), PAR protein from Arachis ipaensis (e.g. SEQ ID NO: 45 or SEQ ID NO: 49), PAR protein from Brachypodium distachyon (e.g. UniProtKB: I1J0D9), PAR protein from Brassica oleracea var. oleracea (e.g. UniProtKB: A0A0D3A1Q6 or A0A0D3A1Q3), PAR protein from Brassica campestris (e.g. UniProtKB: A0A398AHT1), PAR protein from Brassica rapa (e.g. SEQ ID NO: 47), PAR protein from Brassica rapa subsp. Pekinensis (e.g. UniProtKB: M4D574 or M4D571), PAR protein from Brassica oleracea (e.g. UniProtKB: A0A3P6ESB1 or A0A3P6F726), PAR protein from Brassica campestris (e.g. UniProtKB: A0A3P5ZMM3 or A0A3P5Z1M1), PAR protein from Cajanus cajan (e.g. SEQ ID NO: 46), PAR protein from Capsella rubella (e.g. UniProtKB: R0H2J1 or ROHOC2), PAR protein from Cephalotus follicularis (e.g. UniProtKB: A0A1Q3CSK1), PAR protein from Cicer arietinum (e.g. UniProtKB: A0A3Q7YBZ1, A0A1S2YZL9, A0A3Q7Y0Z6 or A0A1S2YZM6; or SEQ ID NO: 55, 56 or 57), PAR protein in Cichorium endivia (e.g. SEQ ID NO: 39), PAR protein from Cucumis sativus (e.g. UniProtKB: A0A0A0KGW4 or A0A0A0L0X7), PAR protein from Cucumis melo (e.g. UniProtKB: A0A1S3BLF2 or A0A1S3B298), PAR protein from Cucumis sativus (e.g. UniProtKB: A0A0A0KAW8), PAR protein from Cucurbita moschata (e.g. SEQ ID NO: 43), PAR protein from Cuscuta campestris (e.g. UniProtKB: A0A484MGR1), PAR protein from Dendrobium catenatum (e.g. UniProtKB: A0A210V7N9, A0A2I0X2T2 or A0A2I0W0Q8), PAR protein from Dorcoceras hygrometricum (e.g. UniProtKB: A0A2Z7D3Y1), PAR protein from Eutrema salsugineum (e.g. UniProtKB: V4LSH0; or SEQ ID NO: 44), PAR protein from Fagus sylvatica (e.g. UniProtKB: A0A2N9E5Y5, A0A2N9HAB9, or A0A2N9H993), PAR protein from Genlisea aurea (e.g. UniProtKB: S8E1M6), PAR protein from Glycine max (e.g. SEQ ID NO: 51, 52, 53 or 54), PAR protein from Gossypium hirsutum (e.g. UniProtKB: A0A1U8LDU9), PAR protein from Helianthus annuus (e.g. SEQ ID NO: 21), PAR protein from Hevea brasiliensis (e.g. SEQ ID NO: 42), PAR protein in Hieracium aurantiacum (e.g. SEQ ID NO: 40), PAR protein from Juglans regia (e.g. UniProtKB: A0A214E6B1), PAR protein from Lactuca sativa (e.g. UniProtKB: A0A2J6KZF7; or SEQ ID NO: 22), PAR protein from Lagenaria siceraria (e.g. SEQ ID NO: 48), PAR protein from Medicago truncatula (e.g. UniProtKB: G7K024), PAR protein from Morus notabilis (e.g. UniProtKB: W9SMY3 or W9SMQ7), PAR protein from Mucuna pruriens (e.g. UniProtKB: A0A371ELJ8), PAR protein from Nicotiana attenuata (e.g. UniProtKB: A0A1J61Q16), PAR protein from Nicotiana sylvestris (e.g. UniProtKB: A0A1U7VXJ0), PAR protein from Nicotiana tabacum (e.g. UniProtKB: A0A1S4A651 or A0A1S3YHQ2), PAR protein from Oryza sativa subsp. Japonica (e.g. UniProtKB: B9FGH8), PAR protein from Oryza barthii (e.g. UniProtKB: A0A0D3FVVX3), PAR protein from Panicum miliaceum (e.g. UniProtKB: A0A3L6Q010 or A0A3L6T1D6), PAR protein from Parasponia andersonii (e.g. UniProtKB: A0A2P5BM15), PAR protein from Populus alba (e.g. UniProtKB: A0A4U5PSY9), PAR protein from Populus trichocarpa (e.g. UniProtKB: B9H661), PAR protein from Punica granatum (e.g. UniProtKB: A0A2I0IBB9, A0A218XB85 or A0A218W102), PAR protein from Senecio cambrensis (e.g. SEQ ID NO: 41), PAR protein from Prunus persica (e.g. SEQ ID NO: 50), PAR protein from Trema orientale (e.g. UniProtKB: A0A2P5EB04), PAR protein from Trifolium pratense (e.g. UniProtKB: A0A2K3N851), PAR protein from Trifolium subterraneum (e.g. UniProtKB: A0A2Z6MYD3 or A0A2Z6MDR7), PAR protein from Trifolium pratense (e.g. UniProtKB: A0A2K3PR44), PAR protein from Vitis vinifera (e.g. UniProtKB: A0A438C778, A0A438ESC4 or A0A438DBR4) and PAR protein from Zea mays (e.g. UniProtKB: A0A1D6HF46, B6UAC5, A0A3L6F4S1, A0A3L6EMC6, A0A3L6EMC6, K7UHQ6 or A0A1D6KHZ4). Such gene may also encode fora PAR protein selected from the group consisting of: PAR protein from Actinidia chinensis (UniProtKB: A0A2R6S2S9), PAR protein from Beta vulgaris (UniProtKB: XP_010690656.1), PAR protein from Solanum tuberosum (UniProtKB: XP_015159151.1), PAR protein from Solanum lycopersicum (UniProtKB: A0A3Q7GXB3), PAR protein from Capsicum baccatum (UniProtKB: A0A2G2WJR7), PAR protein from Solanum melongena (UniProtKB: AVC18974.1), PAR protein from Glycine soja (GeneBank accession: XP_028201014.1, XP_006596577.1 or UniprotKB:A0A445M3M6), PAR protein from Arachis hypogaea (UniProtKB: A0A444WUX5), PAR protein from Phaseolus vulgaris (UniProtKB: V7CIF6), PAR protein from Daucus carota (GeneBank accession: XP_017245413.1), PAR protein from Triticum aestivum (UniProtKB: A0A3B6RP64), PAR protein from Oryza sativa subsp. indica (UniProtKB: A2YH63), PAR protein from Oryza sativa subsp. japonica (UniProtKB: Q5Z7P5) and PAR protein from Theobroma cacao (UniProtKB: A0A061DL63). The invention encompasses these orthologous genes, their promoter sequences, coding sequences (including cDNA and mRNA sequences) and 3′UTRs.

The nucleic acid of the invention may be, but is not limited to, DNA, such as genomic DNA, cDNA or RNA such as mRNA. Preferably, a nucleic acid of the invention is an isolated nucleic acid. Preferably, a variant nucleic acid as defined herein preferably comprises at least about 60%, 70%, 75%, 80%, 85%, 90%, 92%, 95%, 97%, 98%, 99% or more nucleotide sequence identity to any one of the sequences of SEQ ID NO: 2, 3, 4, 5, 7, 8, 9, 10, 12, 13, 14 and 15, and/or to any one of the sequences encoding SEQ ID NO: 1, 6, and 11, or the complements thereof, respectively, preferably when aligned pairwise using e.g. the Needleman and Wunsch algorithm (global sequence alignment) with default parameters. For example, a variant of a coding sequence of SEQ ID NO: 3 preferably comprises at least 60%, 70%, 75%, 80%, 85%, 90%, 92%, 95%, 97%, 98%, 99% or more nucleotide sequence identity to SEQ ID NO: 3; a variant of a coding sequence of SEQ ID NO: 5 preferably comprises at least about 60%, 70%, 75%, 80%, 85%, 90%, 92%, 95%, 97%, 98%, 99% or more nucleotide sequence identity to SEQ ID NO: 5; and so on.

Preferably, the variant differs from any one of SEQ ID NO: 2, 3, 4, 5, 7, 8, 9, 10, 12, 13, 14 and 15, and of the sequences encoding SEQ ID NO: 1, 6, and 11, or complements thereof, by one or more nucleotide deletions, insertions and/or replacements and includes a natural and/or synthetic/artificial variant. A “natural variant” is a variant found in nature, e.g. in other Taraxacum species or in other plants. Preferably a variant is a nucleotide sequence (gene, promoter sequence or coding sequence) from a different plant species, e.g. from a different Taraxacum species than Taraxacum officinale sensu lato, e.g. different cultivars, accessions or breeding lines. Said variant may also be found in and/or isolated from plants other than those belonging to the genus Taraxacum.

As indicated herein, the nucleic acid of the invention also encompasses a fragment of the defined gene, promoter or coding sequence of the Par or par allele, or any variant thereof, as defined herein. A “fragment” comprises or consists of a contiguous nucleotide sequence of any one of SEQ ID NO: 2, 3, 4, 5, 7, 8, 9, 10, 12, 13, 14 and 15, and/or of any one of the sequences encoding SEQ ID NO: 1, 6, and 11, or a variant thereof, such as at least about 10, 12, 15, 18, 20, 30, 50, 100, 150, 200, 250, 300, 500, 1000, 2000 or more contiguous nucleotides, or its complement that is preferably capable of hybridizing to said sequence. In an embodiment, such fragment may be functional in parthenogenesis (preferably capable of inducing parthenogenesis) as defined herein. In another embodiment such fragment may not be functional in parthenogenesis, but may be associated with parthenogenesis for instance because the fragment may hybridize to a sequence that is functional in parthenogenesis, and may therefore be indicative thereof. Such fragment may be useful as e.g. PCR primer or hybridization probe and can thereby be used as a genetic marker for use in a mapping assay or in a molecular assay and/or for identifying and/or isolating Par or par alleles from other plants.

Preferably, the nucleic acid of the invention comprises or consists of a regulatory sequence, preferably the promoter sequence, of a gene encoding a PAR protein as defined herein, wherein said regulatory sequence, preferably promoter sequence, comprises a nucleic acid insert, preferably a double-stranded DNA insert, wherein said insert has a length of between 50 and 2000 bp, between 100 and 1900 bp, between 200 and 1800 bp, between 300 and 1700 bp, between 400 and 1600 bp, between 500 and 1500 bp, between 600 and 1400 bp, between 1000 and 1400, between 1200 and 1400, or between 1300 and 1400 bp. Even more preferably, said insert has a length of about 1300 bp. Preferably, the insert is associated with, and optionally is functional in the parthenogenesis phenotype as defined herein. Preferably, said insert is localized within a promoter sequence that is localized directly upstream (3′) of the sequence encoding the PAR protein, preferably such that the distance between the 3′ end of said insert and the start codon of the sequence encoding the PAR protein is between 50-200 bp, preferably about 50, 60, 70, 80, 90, 100, 110, 120, 130, 140, 150, 160, 170, 180, 190 or 200 bp, most preferably about 102 bp. Preferably, said insert is localized such that the 3′ end nucleotide of the insert is at a position that is homologous to the position of nucleotide 1798 of SEQ ID NO: 2 and/or of nucleotide 1798 of SEQ ID NO: 5. Preferably, said insert is devoid of an open reading frame. Even more preferably said insert is a Miniature Inverted-Repeat Transposable Elements (MITE) or MITE-like sequence, wherein said MITE or MITE-like sequence is a non-autonomous element characterized that contains an internal sequence devoid of an open reading frame, that is flanked by terminal inverted repeats (TIRs) which in turn are flanked by small direct repeats (target site duplications). For a further description of MITE, TIR and sequences, referred is to Guo et al, Scientific Reports. 2017 Jun. 1; 7(1):2634 which is incorporated herein by reference. Said insert, preferably said MITE or MITE-like sequence, may have at least about 50%, 60%, 70%, 80%, 90%, 95%, 98%, 99% or more identity to SEQ ID NO: 60. Preferably, said insert is associated with, and optionally is functional in the parthenogenesis phenotype as defined herein. In a further preferred embodiment, the nucleic acid of the invention comprises or consists of a regulatory sequence, preferably promoter sequence, encompassing said insert at the position as defined herein above. Preferably, the nucleic acid of the invention comprises or consists of a sequence encoding a PAR protein as defined herein operably linked to said promoter sequence, wherein preferably said promoter sequence is localized directly upstream of the sequence encoding the PAR protein. Optionally, said nucleic acid of the invention may comprise one or more further transcription regulatory sequences.

In an embodiment, the nucleic acids of the invention may originate from Taraxacum lines (e.g. Taraxacum officinale sensu lato) or from other species.

In one embodiment of the nucleic acid of the invention is from a different origin than from Taraxacum or Taraxacum officinale sensu lato.

In one embodiment, the invention encompasses a homologous or orthologous Par allele derived from a plant wherein parthenogenesis is present, such as a wild or cultivated plant and/or from other plants. Such homologue or orthologue can be easily isolated by using the provided nucleotide sequences or part thereof as primers or probes. For example, moderate or stringent nucleic acid hybridization methods can be used, using e.g. fragments of the nucleotide sequences as defined herein, or complements thereof. Variants can also be isolated from other wild or cultivated apomictic or non-apomictic plants (and/or from other plants, using known methods such as PCR, stringent hybridization methods, and the like. Thus, variants of any one of SEQ ID NO: 2, 3, 4, 5, 7, 8, 9, 10, 12, 13, 14 and 15, and/or of the sequences encoding SEQ ID NO: 1, 6, and 11, also include nucleic acids found naturally (or in a nature) in other Taraxacum plants, lines or cultivars, and/or found naturally in other plants.

For optimal expression in a host or host cell, the coding sequence as taught herein can be codon-optimized by adapting the codon usage to that most preferred in plant genes, particularly to genes native to the plant genus or species of interest (Bennetzen and Hall, 1982, J. Biol. Chem. 257, 3026-3031; Itakura et al., 1977 Science 198, 1056-1063) using available codon usage Tables (e. g. more adapted towards expression in the pant of interest). Codon usage Tables for various plant species are published for example by Ikemura (1993, In “Plant Molecular Biology Labfax”, Croy, ed., Bios Scientific Publishers Ltd.) and Nakamura et al. (2000, Nucl. Acids Res. 28, 292.) and in the major DNA sequence databases (e.g. EMBL at Heidelberg, Germany). Accordingly, a synthetic DNA sequence can be constructed so that the same or substantially the same protein can be produced using said synthetic DNA sequence. Several techniques for modifying the codon usage to that preferred by the host cells can be found in patent and scientific literature. The exact method of codon usage modification is not critical for this invention.

Small modifications to any one of SEQ ID NO: 2, 3, 4, 5, 7, 8, 9, 10, 12, 13, 14 and 15, and/or of the sequences encoding SEQ ID NO: 1, 6, and 11, or variants thereof, can be routinely made, i.e., by random or targeted mutagenesis (for instance by chemical mutagenesis or CRISPR-endonuclease mediated mutagenesis). More profound modifications to said sequences as taught herein can be routinely done by de novo DNA synthesis of a desired sequence using available techniques.

In an embodiment, the nucleic acid of the invention can be modified so that the N-terminus of the protein of the invention encoded by said nucleic acid has an optimum translation initiation context, by adding or deleting one or more amino acids at the N-terminal end of the protein. Often it is preferred that the protein of the invention, to be expressed in plants cells, starts with a Met-Asp or Met-Ala dipeptide for optimal translation initiation. An Asp or Ala codon may thus be inserted following the existing Met, or the second codon, Val, can be replaced by a codon for Asp (GAT or GAC) or Ala (GCT, GCC, GCA or GCG). The nucleotide sequence may also be modified to remove illegitimate splice sites.

In one embodiment, the nucleic acid of the invention may have a (genetically) dominant function, preferably provided by (over)expressing a functional protein having the amino acid sequence SEQ ID NO: 1, or a variant or functional fragment thereof, such as an orthologue or fragment thereof found in another plant (i.e. other than Taraxacum or Taraxacum officinale sensu lato).

Preferably, the nucleic acid of the invention encodes a protein or functional fragment(s) thereof which, when produced in the plant, is functional and induces and/or enhances parthenogenesis. For example, when the nucleic acid comprising SEQ ID NO: 3 or 5, or variant or fragment thereof, is expressed (transcribed and translated) and suitable amounts of the protein of the invention is made in the appropriate plant tissues, the parthenogenetic effect is significantly enhanced as compared to plants that only differ in that they lack said nucleic acid. Functionality can also be easily tested by (over)expressing the nucleic acid of the invention in a suitable host plant, such as a non-parthenogenetic Taraxacum line, and analyzing the parthenogenetic effect of the transformant in a bioassay, e.g. as described in the Example 2. Functionality of said nucleic acid is preferably assessed by comparing a test plant wherein one or more of these nucleic acids is (over)expressed to a control plant which only differs from the test plant in that the control plants lacks (over)expression of said nucleic acid. Alternatively, silencing or disruption of the nucleic acid of the invention that is associated with parthenogenesis may lead to loss-of-function, i.e. to reduced parthenogenesis.

The nucleic acid of the invention can be used to generate a vector or plasmid for expressing the protein of the invention in a suitable host cell, or for silencing one or more endogenous parthenogenesis genes or gene families. Hence, constructs, vectors and/or plasmids comprising a nucleic acid of the invention and/or silencing constructs are also encompassed by the present invention.

### Amino Acid Sequences According to the Invention

The invention provides for a PAR protein as defined herein. The invention also provides for a protein that is associated with parthenogenesis in plants, wherein said protein:

a) is encoded by the nucleic acid of the invention;

b) has an amino acid sequence of SEQ ID NO: 1, 6 or 11;

c) is a variant of a) and/or b); and/or

d) is a fragment of any one of a)-c),

wherein preferably said protein is functional in parthenogenesis.

In one embodiment, the protein of the invention is:

a) is encoded by the nucleic acid of any one of SEQ ID NO: 3, 8 or 13;

b) has an amino acid sequence of SEQ ID NO: 1, 6 or 11;

c) is a variant of a) and/or b); and/or

d) is a fragment of any one of a)-c),

wherein preferably the protein of the invention is suitable for inducing parthenogenesis.

In one embodiment, the protein of the invention is:

a) is encoded by the nucleic acid of SEQ ID NO: 3 or 5;

b) has an amino acid sequence of SEQ ID NO: 1;

c) is a variant of a) and/or b); and/or

d) is a fragment of any one of a)-c),

wherein preferably the protein of the invention is suitable for inducing parthenogenesis. The variant preferably is a PAR protein as defined herein. Preferably the protein or protein fragment is encoded by a nucleic acid of SEQ ID NO: 3 or 5, or variant and/or fragment thereof, or such protein comprises SEQ ID NO: 1, or variant and/or fragment thereof. Preferably said variant comprises or consists of an amino acid sequence that has at least about 50%, 60%, 70%, 80%, 90%, 95%, 98%, 99% or more identity to SEQ ID NO: 1, 6 or 11, respectively, preferably when aligned pairwise using e.g. the Needleman and Wunsch algorithm (global sequence alignment) with default parameters. A variant differs from the provided sequence by one or more amino acid residue deletions, insertions and/or replacements and include natural and/or synthetic/artificial variants. A variant of a protein having an amino acid encoded by a nucleic acid of the invention, preferably a variant of a protein encoded by any one of SEQ ID NO: 3, 5, 8, 10, 13, 15, or variant of a protein having an amino acid sequence of any one of SEQ ID NO: 1, 6 or 11, may be a homologue or orthologue. Such an orthologous protein encompassed by the present invention may be, but is not limited to, any one of the PAR proteins selected from the group consisting of: PAR protein from Ananas comosus (e.g. UniProtKB: A0A199URK4), PAR protein from Apostasia shenzhenica (e.g. UniProtKB: A0A210AZW3), PAR protein from Arabidopsis thaliana (e.g. UniProtKB: Q8GXP9, A0A178V2S4, 081793, A0A178V1Q3, A0MFC1, O81801), PAR protein from Arabidopsis lyrata subsp. Lyrata (e.g. UniProtKB: D7MC52 or D7MCE8), PAR protein from Arachis ipaensis (e.g. SEQ ID NO: 45 or SEQ ID NO 49), PAR protein from Brachypodium distachyon (e.g. UniProtKB: I1J0D9), PAR protein from Brassica oleracea var. oleracea (e.g. UniProtKB: A0A0D3A1Q6 or A0A0D3A1Q3), PAR protein from Brassica campestris (e.g. UniProtKB: A0A398AHT1), PAR protein from Brassica rapa (e.g. SEQ ID NO: 47), PAR protein from Brassica rapa subsp. Pekinensis (e.g. UniProtKB: M4D574 or M4D571), PAR protein from Brassica oleracea (e.g. UniProtKB: A0A3P6ESB1 or A0A3P6F726), PAR protein from Brassica campestris (e.g. UniProtKB: A0A3P5ZMM3 or A0A3P5Z1M1), PAR protein from Cajanus cajan (e.g. SEQ ID NO: 46), PAR protein from Capsella rubella (e.g. UniProtKB: R0H2J1 or R0H0C2), PAR protein from Cephalotus follicularis (e.g. UniProtKB: A0A1Q3CSK1), PAR protein from Cicer arietinum (e.g. UniProtKB: A0A3Q7YBZ1, A0A1S2YZL9, A0A3Q7Y0Z6 or A0A1S2YZM6; or SEQ ID NO: 55, 56 or 57), PAR protein in Cichorium endivia (e.g. SEQ ID NO: 39), PAR protein from Cucumis sativus (e.g. UniProtKB: A0A0A0KGW4 or A0A0A0L0X7), PAR protein from Cucumis melo (e.g. UniProtKB: A0A1S3BLF2 or A0A1S3B298), PAR protein from Cucumis sativus (e.g. UniProtKB: A0A0A0KAW8), PAR protein from Cucurbita moschata (e.g. SEQ ID NO: 43), PAR protein from Cuscuta campestris (e.g. UniProtKB: A0A484MGR1), PAR protein from Dendrobium catenatum (e.g. UniProtKB: A0A210V7N9, A0A210X2T2 or A0A210W0Q8), PAR protein from Dorcoceras hygrometricum (e.g. UniProtKB: A0A2Z7D3Y1), PAR protein from Eutrema salsugineum (e.g. UniProtKB: V4LSH0; or SEQ ID NO: 44), PAR protein from Fagus sylvatica (e.g. UniProtKB: A0A2N9E5Y5, A0A2N9HAB9, or A0A2N9H993), PAR protein from Genlisea aurea (e.g. UniProtKB: S8E1M6), PAR protein from Glycine max (e.g. SEQ ID NO: 51, 52, 53 or 54), PAR protein from Gossypium hirsutum (e.g. UniProtKB: A0A1U8LDU9), PAR protein from Helianthus annuus (e.g. SEQ ID NO: 21), PAR protein from Hevea brasiliensis (e.g. SEQ ID NO: 42), PAR protein in Hieracium aurantiacum (e.g. SEQ ID NO: 40), PAR protein from Juglans regia (e.g. UniProtKB: A0A214E6B1), PAR protein from Lactuca sativa (e.g. UniProtKB: A0A2J6KZF7; or SEQ ID NO: 22), PAR protein from Lagenaria siceraria (e.g. SEQ ID NO: 48), PAR protein from Medicago truncatula (e.g. UniProtKB: G7K024), PAR protein from Morus notabilis (e.g. UniProtKB: W9SMY3 or W9SMQ7), PAR protein from Mucuna pruriens (e.g. UniProtKB: A0A371ELJ8), PAR protein from Nicotiana attenuata (e.g. UniProtKB: A0A1J61Q16), PAR protein from Nicotiana sylvestris (e.g. UniProtKB: A0A1 U7VXJ0), PAR protein from Nicotiana tabacum (e.g. UniProtKB: A0A1S4A651 or A0A1S3YHQ2), PAR protein from Oryza sativa subsp. Japonica (e.g. UniProtKB: B9FGH8), PAR protein from Oryza barthii (e.g. UniProtKB: A0A0D3FWX3), PAR protein from Panicum miliaceum (e.g. UniProtKB: A0A3L6Q010 or A0A3L6T1D6), PAR protein from Parasponia andersonii (e.g. UniProtKB: A0A2P5BM15), PAR protein from Populus alba (e.g. UniProtKB: A0A4U5PSY9), PAR protein from Populus trichocarpa (e.g. UniProtKB: B9H661), PAR protein from Punica granatum (e.g. UniProtKB: A0A210IBB9, A0A218XB85 or A0A218W102), PAR protein from Senecio cambrensis (e.g. SEQ ID NO: 41), PAR protein from Prunus persica (e.g. SEQ ID NO: 50), PAR protein from Trema orientale (e.g. UniProtKB: A0A2P5EB04), PAR protein from Trifolium pratense (e.g. UniProtKB: A0A2K3N851), PAR protein from Trifolium subterraneum (e.g. UniProtKB: A0A2Z6MYD3 or A0A2Z6MDR7), PAR protein from Trifolium pratense (e.g. UniProtKB: A0A2K3PR44), PAR protein from Vitis vinifera (e.g. UniProtKB: A0A438C778, A0A438ESC4 or A0A438DBR4) and PAR protein from Zea mays (e.g. UniProtKB: A0A1D6HF46, B6UAC5, A0A3L6F4S1, A0A3L6EMC6, A0A3L6EMC6, K7UHQ6 or A0A1D6KHZ4). Such orthologous protein may also be a PAR protein selected from the group consisting of: PAR protein from Actinidia chinensis (UniProtKB: A0A2R6S2S9), PAR protein from Beta vulgaris (UniProtKB: XP_010690656.1), PAR protein from Solanum tuberosum (UniProtKB: XP_015159151.1), PAR protein from Solanum lycopersicum (UniProtKB: A0A3Q7GXB3), PAR protein from Capsicum baccatum (UniProtKB: A0A2G2WJR7), PAR protein from Solanum melongena (UniProtKB: AVC18974.1), PAR protein from Glycine soja (GeneBank accession: XP_028201014.1, XP_006596577.1 or UniprotKB: A0A445M3M6), PAR protein from Arachis hypogaea (UniProtKB: A0A444WUX5), PAR protein from Phaseolus vulgaris (UniProtKB: V7CIF6), PAR protein from Daucus carota (GeneBank accession: XP_017245413.1), PAR protein from Triticum aestivum (UniProtKB: A0A3B6RP64), PAR protein from Oryza sativa subsp. indica (UniProtKB: A2YH63), PAR protein from Oryza sativa subsp. japonica (UniProtKB: Q5Z7P5) and PAR protein from Theobroma cacao (UniProtKB: A0A061DL63).

Therefore, the variant of the protein of SEQ ID NO: 1 encompassed by the invention may be, but is not limited to, any one of the orthologues PAR proteins as defined herein.

The PAR protein of the invention, and/or the variant of the protein having SEQ ID NO: 1, 6 or 11, may be capable of inducing parthenogenesis when present in a plant or plant cell. The variant of the protein can be an endogenous or non-endogenous protein of said plant or plant cell. Optionally, the PAR protein of the invention and/or the variant of the protein having SEQ ID NO: 1, 6 or 11, is capable of inducing parthenogenesis when expression of the protein has been altered, preferably increased. Preferably, such altered expression, preferably increased expression, is within the egg cell. Altered or increased expression may be de novo expression of said protein in a plant or plant cell, or maybe increased expression of an endogenous protein in a plant or plant cell. The person skilled in the art is aware of ways to increase expression of a protein. De novo expression of the protein in a plant or plant cell may be induced by e.g., transfection of the plant or plant cell with a construct or vector encoding the protein, introgression of gene encoding the protein into progeny of the plant or plant cell, and/or modifying an endogenous sequence resulting in a sequence encoding said protein for instance by genetic modification. Optionally, such construct or vector comprises a sequence encoding the PAR protein operably linked to an egg cell promoter. The person skilled in the art is aware of egg cell promoters. Exemplary egg cell promoters that are capable of driving expression in egg cells of plants include, but are not limited to the promoter of the egg-cell specific gene ECI 0.1, ECI 0.2, ECI 0.3, EC1.4, or ECI.5 (see, e.g. Sprunck et al. Science, 338:1093-1097 (2012); AT2G21740; Steffen et al, Plant Journal 51: 281-292 (2007)), the Arabidopsis DD45 promoter (Ohnishi et al. PlantPhysiology 165: 1533-1543 (2014)). Preferably, a construct or vector of the invention comprises a sequence encoding the PAR protein operably linked to a regulatory sequence, preferably a promoter sequence, comprising a nucleic acid insert, preferably a double-stranded DNA insert, wherein said insert has a length of between 50 and 2000 bp, between 100 and 1900 bp, between 200 and 1800 bp, between 300 and 1700 bp, between 400 and 1600 bp, between 500 and 1500 bp, between 600 and 1400 bp, between 1000 and 1400, between 1200 and 1400, or between 1300 and 1400 bp. Even more preferably, said insert has a length of about 1300 bp. Preferably, the insert is associated with, and optionally is functional in the parthenogenesis phenotype as defined herein. Preferably, said insert is localized within a promoter sequence that is localized directly upstream (3′) of the sequence encoding the PAR protein, preferably such that the distance between the 3′ end of said insert and the start codon of the sequence encoding the PAR protein is between 50-200 bp, preferably about 50, 60, 70, 80, 90, 100, 110, 120, 130, 140, 150, 160, 170, 180, 190 or 200 bp, most preferably about 102 bp. Preferably, said insert is localized such that the 3′ end nucleotide of the insert is at a position that is homologous to the position of nucleotide 1798 of SEQ ID NO: 2 and/or of nucleotide 1798 of SEQ ID NO: 5. Preferably, said insert is devoid of an open reading frame. Even more preferably said insert is a Miniature Inverted-Repeat Transposable Elements (MITE) or MITE-like sequence, wherein said MITE or MITE-like sequence is a non-autonomous element characterized that contains an internal sequence devoid of an open reading frame, that is flanked by terminal inverted repeats (TIRs) which in turn are flanked by small direct repeats (target site duplications). For a further description of MITE, TIR and sequences, referred is to Guo et al, Scientific Reports. 2017 Jun. 1; 7(1):2634 which is incorporated herein by reference. Said insert, preferably said MITE or MITE-like sequence, may have at least about 50%, 60%, 70%, 80%, 90%, 95%, 98%, 99% or more identity to SEQ ID NO: 60. Preferably, said insert is associated with, and optionally is functional in the parthenogenesis phenotype as defined herein. In a further preferred embodiment, the construct or vector of the invention comprises or consists of a regulatory sequence, preferably promoter sequence, encompassing said insert at the position as defined herein above. Preferably, the construct or vector comprises or consists of a sequence encoding a PAR protein as defined herein operably linked to said promoter sequence, wherein preferably said promoter sequence is localized directly upstream of the sequence encoding the PAR protein. Optionally, said construct or vector of the invention may comprise one or more further transcription regulatory sequences.

In addition, or alternatively, such construct or vector comprises a sequence encoding the PAR protein operably linked to the promoter of SEQ ID NO: 2. Altered or increased expression of an endogenous protein may be induced by modifying one or more regulatory sequence operably linked to the coding sequence. For instance, the promoter sequence operably linked to the sequence encoding the protein may be modified, for instance by genetic modification. In a preferred embodiment, the insert as defined herein above is introduced in the promoter sequence, preferably at a position as defined herein above. Such functionality of being capable of inducing parthenogenesis may be assessed by using a suitable test for functionality in parthenogenesis of a nucleic acid encoding said variant, as described herein. The protein of the invention may be an isolated protein.

“Natural variants” are those found in nature, e.g. in cultivated or wild lettuce plants and/or other plants. Also included is a fragment, i.e. a non-full length peptide of the protein of the invention, preferably functional fragment, i.e. which is capable of inducing parthenogenesis when expressed in a suitable host plant. Fragments of the proteins as taught herein include peptides comprising or consisting of at least about 10, 20, 30, 40, 50, 100, 150, 200, 250 or more contiguous amino acid sequences encoded by the nucleic acid of the invention, especially comprising or consisting of at least about 10, 20, 30, 40, 50, 100, 150, 200, 250 or more contiguous amino acids of SEQ ID NO: 1, 6 or 11, or variant thereof (as defined herein). Sequences found in nature are also indicated herein as “wild type”.

The protein of the invention maybe isolated from natural sources, synthesized de novo by chemical synthesis (using e.g. a peptide synthesizer such as supplied by Applied Biosystems) or produced by recombinant host cells by expressing the nucleotide sequence as taught herein encoding the protein of the invention. The protein of the invention may also be produced by expression from a nucleic acid of the invention as defined herein.

Protein variants may comprise conservative amino acid substitutions within the categories basic (e. g. Arg, His, Lys), acidic (e. g. Asp, Glu), nonpolar (e. g. Ala, Val, Trp, Leu, Ile, Pro, Met, Phe, Trp) or polar (e. g. Gly, Ser, Thr, Tyr, Cys, Asn, Gln). In addition non-conservative amino acid substitutions fall within the scope of the invention.

Chimeric proteins, such as proteins composed of domains from different sources such as an N-terminal of the protein of SEQ ID NO: 1, 6 or 11 (e.g. obtained from Taxaracum or plant species X) and a middle domain and/or C-terminal domain of variant of SEQ ID NO: 1, 6 or 11 (e.g. obtained from Taxaracum or plant species Y or another plant species) are also encompassed herein. Preferably, a chimeric protein is composed of domains from at least two orthologous proteins. Such chimeric protein may have improved functionality, e.g. the sense that it may more efficiently confer parthenogenesis than the native protein when expressed in the plant host.

Also all nucleotide sequences (RNA, cDNA, genomic DNA, etc.) encoding the protein, protein variant or protein fragment of the invention are encompassed by the present invention. Due to the degeneracy of the genetic code various nucleotide sequences may encode the same amino acid sequence.

Parthenogenetic Plants and Methods of Making these

In a further aspect, the present invention relates to plants (including e.g. plant cells, organs, seeds and plant parts), and methods of making plants, which show modified parthenogenesis, optionally transgenic plants having modified, preferably induced, parthenogenesis as compared to a native or unmodified plant. Such plants can be made using different methods, e.g. as described further herein. Preferably, the plant of the invention is obtained by a technical means, preferably by a method as described herein. Such technical means are well-known to the skilled person and include genetic modifications, such as e.g. at least one of random mutagenesis, targeted mutagenesis and nucleic acid insertions.

Preferably, the plant of the invention is not obtained by an essentially biological process. Preferably, the plant of the invention is not exclusively obtained by an essentially biological process. Preferably, the plant of the invention is not obtained, preferably not directly obtained, by any essentially biological process that introduces parthenogenesis in a plant. Preferably, the plant of the invention is not exclusively obtained by any essentially biological process that introduces parthenogenesis in a plant. Preferably, the plant of the invention is not a naturally occurring plant, i.e. is not a plant that occurs in nature.

In particular, the invention provides for a method for producing a parthenogenetic plant, comprising the steps of:
a) introducing in one or more plant cells a nucleic acid of the invention, and/or its derived product, that is capable of inducing parthenogenesis and/or is functional in parthenogenesis;
b) optionally selecting a plant cell comprising said nucleic acid, wherein preferably said nucleic acid is integrated in the genome of said plant cell; and
c) regenerating a plant from said plant cell,
   wherein preferably, said nucleic acid of the invention encodes, or is operably linked to a sequence encoding, a PAR protein as defined herein that is functional in parthenogenesis, and/or is any one of SEQ ID NO: 2-5, or encoding a protein of SEQ ID NO: 1, or variant or fragment thereof.

The invention further provides a method for producing an apomictic plant, comprising the steps of:
a) introducing in one or more plant cells capable of apomeiosis a nucleic acid of the invention, and/or its derived product, that is capable of inducing parthenogenesis;
b) optionally selecting a plant cell comprising said nucleic acid, wherein preferably said nucleic acid is integrated in the genome of said plant cell; and
c) regenerating a plant from said plant cell,
   wherein preferably, said nucleic acid of the invention encodes, or is operably linked to a sequence encoding, a PAR protein as defined herein that is functional in parthenogenesis, and/or is any one of SEQ ID NO: 2-5, or encoding a protein of SEQ ID NO: 1, or variant or fragment thereof. A plant cell capable of apomeiosis may be obtained by introduction a nucleic acid capable of conferring apomeiosis. Optionally said nucleic acid is introduced in a plant cell before, together or after the introduction of a nucleic acid of the present invention.

The nucleic acid of the invention can be introduced in one or more plant cells by transforming, introgression, somatic hybridization and/or protoplast fusion. Such nucleic acid may be an exogenous nucleic acid, i.e. a nucleic acid not occurring in said plant cell in nature.

The nucleic acid of the invention can be introduced in one or more plant cells by modifying an endogenous nucleic acid to obtain the nucleic acid of the invention. Modification of endogenous genes preferably comprises random or targeted mutation of one or more nucleotides, or the insertion or deletion of a short or larger sequence for instance by homologous recombination, in the coding sequence and/or in the regulatory and/or promoter sequence in order to alter expression of an endogenous protein. Such method preferably results in the modification of one or more endogenous par alleles into a Par allele as defined herein. Random mutagenesis may be, but is not limited to, chemical mutagenesis and gamma radiation. Non-limiting examples of chemical mutagenesis include, but are not limited to, EMS (ethyl methanesulfonate), MMS (methyl methanesulfonate), NaN3 (sodium azide) D), ENU (N-ethyl-N-nitrosourea), AzaC (azacytidine) and NQO (4-nitroquinoline 1-oxide). Optionally, mutagenesis systems such as TILLING (Targeting Induced Local Lesions IN Genomics; McCallum et al., 2000, Nat Biotech 18:455, and McCallum et al. 2000, Plant Physiol. 123, 439-442, both incorporated herein by reference) may be used to generate plant lines with a modified gene as defined herein. TILLING uses traditional chemical mutagenesis (e.g. EMS mutagenesis) followed by high-throughput screening for mutations. Thus, plants, seeds and tissues comprising a gene having one or more of the desired mutations may be obtained using TILLING. Targeted mutagenesis is mutagenesis that can be designed to alter a specific nucleotides or nucleic acid sequence, such as but not limited to, oligo-directed mutagenesis, RNA-guided endonucleases (e.g. the CRISPR-technology), TALENs or Zinc finger technology.

Preferably, the modification is a modification in a promoter sequence of a gene that encodes the PAR protein as defined herein. Preferably, the modification introduces or increases the expression of the PAR protein as defined herein. Preferably, the modification introduces or increases the expression of the PAR protein as defined herein in the egg cell.

Therefore, the method of the invention may comprise the steps of:
a) modifying in one or more plant cells a nucleic acid that is, or is operably linked to, a sequence encoding a protein associated with parthenogenesis and/or functional in parthenogenesis, wherein preferably said nucleic acid is within the genome of said one or more plant cells;
b) optionally selecting a plant cell comprising said modified nucleic acid; and
c) regenerating a plant from said plant cell,

wherein preferably, said protein associated with and/or functional in parthenogenesis has an amino acid sequence according to the invention as described herein above. Preferably the nucleic acid to be modified in step a) is an endogenous nucleic acid, preferably comprising or consisting of a nucleotide sequence that is, or is operably linked to a sequence, encoding a PAR protein as defined herein and/or a protein having an amino acid sequence of SEQ ID NO: 1, 6 or 11, or a variant or fragment thereof.

In a particular preferred embodiment, said nucleic acid is the (5′UTR) promoter sequence of the gene encoding the protein associated with parthenogenesis as defined herein. Preferably, said modification is the introduction of a nucleic acid insert, preferably a double-stranded DNA insert, wherein said insert has a length of between 50 and 2000 bp, between 100 and 1900 bp, between 200 and 1800 bp, between 300 and 1700 bp, between 400 and 1600 bp, between 500 and 1500 bp, between 600 and 1400 bp, between 1000 and 1400, between 1200 and 1400, or between 1300 and 1400 bp. Even more preferably, said insert has a length of about 1300 bp. Preferably, the insert is associated with, and optionally is functional in the parthenogenesis phenotype as defined herein. Preferably, said insert is introduced within a promoter sequence that is localized directly upstream (3′) of the sequence encoding the PAR protein, preferably such that the distance between the 3′ end of said insert and the start codon of the sequence encoding the PAR protein is between 50-200 bp, preferably about 50, 60, 70, 80, 90, 100, 110, 120, 130, 140, 150, 160, 170, 180, 190 or 200 bp, most preferably about 102 bp. Preferably, said insert is introduced such that the 3′ end nucleotide of the insert is at a position that is homologous to the position of nucleotide 1798 of SEQ ID NO: 2 and/or of nucleotide 1798 of SEQ ID NO: 5. Preferably, said insert is devoid of an open reading frame. Even more preferably said insert is a Miniature Inverted-Repeat Transposable Elements (MITE) or MITE-like sequence, wherein said MITE or MITE-like sequence is a non-autonomous element characterized that contains an internal sequence devoid of an open reading frame, that is flanked by terminal inverted repeats (TIRs) which in turn are flanked by small direct repeats (target site duplications). For a further description of MITE, TIR and sequences, referred is to Guo et al, Scientific Reports. 2017 Jun. 1; 7(1):2634 which is incorporated herein by reference. Said insert, preferably said MITE or MITE-like sequence, may have at least about 50%, 60%, 70%, 80%, 90%, 95%, 98%, 99% or more identity to SEQ ID NO: 60. Preferably, said insert is associated with, and optionally is functional in the parthenogenesis phenotype as defined herein.

Preferably, the modification of the nucleotide sequence results in an introduced or increased expression of said protein, preferably in the egg cell of the plant regenerated from the plant cell. Preferably, the modified promoter sequence comprises a sequence having at least about 75%, 80%, 85%, 90%, 95%, 96%, 97%, 98%, 99% or 100% sequence identity with SEQ ID NO: 2.

Further, the method of the invention may comprise the steps of:
a) modifying in one or more plant cells capable of apomeiosis a nucleic acid that is, or is operably linked to, a sequence encoding a protein associated with parthenogenesis and/or functional in parthenogenesis, wherein preferably said nucleic acid is within the genome of said one or more plant cells;
b) optionally selecting a plant cell comprising said modified or altered nucleic acid; and
c) regenerating a plant from said plant cell,

wherein preferably, said protein associated with and/or functional in parthenogenesis has an amino acid sequence according to the protein of the invention as described herein above. Preferably the nucleic acid to be modified in step a) is an endogenous nucleic acid, preferably comprising or consisting of a nucleotide sequence that is, or is operably linked to a sequence, encoding a PAR protein as defined herein and/or a protein having an amino acid sequence of SEQ ID NO: 1, 6 or 11, or a variant or fragment thereof. Preferably the nucleic acid to be modified in step a) is an endogenous nucleic acid.

In a particular preferred embodiment, said nucleic acid is the promoter sequence of the gene encoding the protein associated with and/or functional in parthenogenesis as defined herein. Preferably, the modification of the nucleotide sequence results in an introduced or increased expression of said protein, preferably in the egg cell of the plant regenerated from said plant cell. Preferably the modified promoter sequence is a promoter sequence operably linked to the coding sequence of a PAR protein as defined herein. Preferably, said modified promoter sequence is modified to comprise the insert as defined herein above, preferably at the position as defined herein above.

Preferably, the modified promoter sequence comprises a sequence having at least about 75%, 80%, 85%, 90%, 95%, 96%, 97%, 98%, 99% or 100% sequence identity with SEQ ID NO: 2.

The invention also provides for a method of producing an apomictic hybrid seed, comprising the steps of:
a) cross-fertilizing a sexually reproducing first plant with the pollen of a second plant to produce F1 hybrid seeds; and
b) optionally selecting from the said F1 seeds a seed that comprise the apomictic phenotype;
   wherein said first and/or second plant is capable of apomeiosis and wherein said second plant comprises a nucleic acid of the invention, and wherein preferably said selecting is performed by genotyping. Preferably, said second plant comprises a nucleic acid of the invention that is any one of SEQ ID NO: 2-5, or encoding a protein of SEQ ID NO: 1, or variant or fragment thereof.

The nucleic acid of the invention may be comprised in a chimeric gene, genetic construct or nucleic acid vector. In one embodiment of the invention, the nucleic acid of the invention may be used to make a chimeric gene, and/or a vector comprising this nucleic acid for transfer of the nucleic acid into a host cell and production of a functional (preferably capable of inducing parthenogenesis) protein encoded by said nucleic acid in host cells. Vectors for the production of such protein (or protein fragment or variant) in plant cells are herein referred to as i.e. “expression vectors”. Host cells are preferably plant cells.

The construction of a chimeric gene, construct and/or vector for, optionally transient but preferably stable, introduction of a protein-encoding nucleotide sequence into the genome of a host cells is generally known in the art. To generate a chimeric gene for inducing parthenogenesis and/or improving functionality in parthenogenesis, the nucleotide sequence encoding a protein of SEQ ID NO: 1, 6 or 11, or a functional variant and/or functional fragment thereof, may be operably linked to a promoter sequence, suitable for expression in the host cells, using standard molecular biology techniques. The promoter sequence may already be present in a vector so that the nucleotide sequence encoding said protein may simply be inserted into the vector downstream of the promoter sequence. The vector may then be used to transform the host cells and the nucleic acid and/or chimeric gene of the invention may be inserted in the nuclear genome or into the plastid, mitochondrial or chloroplast genome and may be expressed in the host cell using a suitable promoter (e.g., Mc Bride et al., 1995; U.S. Pat. No. 5,693,507). In one embodiment, a nucleic acid and/or chimeric gene of the invention may comprise a suitable promoter for expression in plant cells or microbial cells (e.g. bacteria), operably linked to a nucleotide sequence encoding a protein of the invention, optionally followed by a 3′ nontranslated nucleotide sequence. The coding sequence is optionally preceded by a 5′UTR sequence. Promoter, 3′UTR and/or 5′UTR may, for example, be from a native parthenogenesis gene, or may alternatively be from other sources.

The nucleic acid as taught herein, encoding a protein capable of inducing parthenogenesis as taught herein, can be stably inserted into the nuclear genome of a single plant cell, and the so-transformed plant cell can be used to produce a transformed plant that has an altered phenotype due to the presence of said protein in certain cells at a certain time. In a non-limiting example, a T-DNA vector, comprising the nucleic acid as taught herein encoding a protein functional in parthenogenesis as taught herein, in Agrobacterium tumefaciens can be used to transform the plant cell, and thereafter, a transformed plant can be regenerated from the transformed plant cell using the procedures described, for example, in EP0116718, EP0270822, PCT publication WO84/02913 and published European Patent application EP0242246 and in Gould et al. (1991). The construction of a T-DNA vector for Agrobacterium mediated plant transformation is well known in the art. The T-DNA vector may be either a binary vector as described in EP0120561 and EP0120515 or a co-integrate vector which can integrate into the Agrobacterium Ti-plasmid by homologous recombination, as described in EP0116718. Lettuce transformation protocols have been described in, for example, Michelmore et al. (1987) and Chupeau et al. (1989).

A preferred T-DNA vector contains a promoter operably linked to nucleotide sequence encoding a protein of the invention; e.g. the promoter being operably linked to the nucleotide sequence of SEQ ID NO: 3 or a variant or functional fragment thereof, between T-DNA border sequences, or at least located to the left of the right border sequence. Preferably said promoter is a promoter comprising a nucleic acid insert, preferably a double-stranded DNA insert, wherein said insert has a length of between 50 and 2000 bp, between 100 and 1900 bp, between 200 and 1800 bp, between 300 and 1700 bp, between 400 and 1600 bp, between 500 and 1500 bp, between 600 and 1400 bp, between 1000 and 1400, between 1200 and 1400, or between 1300 and 1400 bp. Even more preferably, said insert has a length of about 1300 bp. Preferably, the insert is associated with, and optionally is functional in the parthenogenesis phenotype as defined herein. Preferably, said insert is localized within a promoter sequence that is localized directly upstream (3′) of the sequence encoding the PAR protein, preferably such that the distance between the 3′ end of said insert and the start codon of the sequence encoding the PAR protein is between 50-200 bp, preferably about 50, 60, 70, 80, 90, 100, 110, 120, 130, 140, 150, 160, 170, 180, 190 or 200 bp, most preferably about 102 bp. Preferably, said insert is localized such that the 3′ end nucleotide of the insert is at a position that is homologous to the position of nucleotide 1798 of SEQ ID NO: 2 and/or of nucleotide 1798 of SEQ ID NO: 5. Preferably, said insert is devoid of an open reading frame. Even more preferably said insert is a Miniature Inverted-Repeat Transposable Elements (MITE) or MITE-like sequence, wherein said MITE or MITE-like sequence is a non-autonomous element characterized that contains an internal sequence devoid of an open reading frame, that is flanked by terminal inverted repeats (TIRs) which in turn are flanked by small direct repeats (target site duplications). For a further description of MITE, TIR and sequences, referred is to Guo et al, Scientific Reports. 2017 Jun. 1; 7(1):2634 which is incorporated herein by reference. Said insert, preferably said MITE or MITE-like sequence, may have at least about 50%, 60%, 70%, 80%, 90%, 95%, 98%, 99% or more identity to SEQ ID NO: 60. Preferably, said insert is associated with, and optionally is functional in the parthenogenesis phenotype as defined herein. In a further preferred embodiment, the T-DNA vector comprises or consists of a regulatory sequence, preferably promoter sequence, encompassing said insert at the position as defined herein above. Preferably, the T-DNA vector comprises or consists of a sequence encoding a PAR protein as defined herein operably linked to said promoter sequence, wherein preferably said promoter sequence is localized directly upstream of the sequence encoding the PAR protein. Optionally, said T-DNA vector may comprise one or more further transcription regulatory sequences.

Border sequences are described in Gielen et al. (1984). Of course, other types of vectors can be used to transform the plant cell, using procedures such as direct gene transfer (as described, for example in EP0223247), pollen mediated transformation (as described, for example in EP0270356 and WO85/01856), protoplast transformation as, for example, described in U.S. Pat. No. 4,684,611, plant RNA virus-mediated transformation (as described, for example in EP0067553 and U.S. Pat. No. 4,407,956), liposome-mediated transformation (as described, for example in U.S. Pat. No. 4,536,475), and other methods.

In a further embodiment, the nucleic acid of the invention may be introduced by somatic hybridization. Somatic hybridization may be done by protoplast fusion (e.g. see Holmes, 2018).

The nucleic acid of the invention can also be integrated in the genome for instance using one or more specific endonucleases (such as a CRISPR-endonuclease/guide RNA complex) for introducing double strand breaks at the appropriate site in the genome and a donor construct comprising the nucleic acid of the invention for integration in the genome. The skilled person knows how to design such CRISPR-endonuclease/guide RNA complex for introducing a double strand break and donor construct suitable for integration (for a review, see Bortesi and Fischer, 2015).

Alternatively, the plant may be transformed by altering the endogenous nucleotide sequence, thereby for instance converting one or more par alleles comprised in the plant into one or more Par alleles, e.g. by random or targeted mutagenesis. Said mutagenesis may involve mutagenesis of the encoding sequence, but may also involve mutagenesis of the regulating sequence, such as the promoter sequence, 5′UTR and/or 3′UTR. Said endogenous 5′UTR promoter nucleotide sequence of a par allele may be modified to comprise the insert as defined herein above, preferably at a position as defined herein above.

Likewise, selection and regeneration of transformed plants from transformed cells is well known in the art. Obviously, for different species and even for different varieties or cultivars of a single species, protocols are specifically adapted for regenerating transformants at high frequency. The invention also encompasses progeny of the transformed plants showing parthenogenesis and comprising the nucleic acid and/or protein of the invention.

Besides transformation of the nuclear genome, also transformation of the plastid genome, preferably chloroplast genome, is included in the invention. One advantage of plastid genome transformation is that the risk of spread of the transgene(s) can be reduced. Plastid genome transformation can be carried out as known in the art, see e.g. Sidorov et al. (1999) or Lutz et al. (2004).

The resulting transformed plant can be used in a conventional plant breeding scheme to produce more transformed plants containing the transgene. Single copy transformants can be selected, using e.g. Southern Blot analysis or PCR based methods or the Invader® Technology assay (Third Wave Technologies, Inc.). Transformed cells and plants can easily be distinguished from non-transformed ones by the presence of the nucleic acid or protein of the invention and/or chimeric gene. The sequences of the plant DNA flanking the insertion site of the transgene can also be sequenced, whereby an “Event specific” detection method can be developed, for routine use. See for example WO0141558, which describes elite event detection kits (such as PCR detection kits) based for example on the integrated sequence and the flanking (genomic) sequence.

The nucleic acid of the invention may be inserted in a plant cell genome so that the inserted coding sequence(s) is downstream (i.e. 3) of, and under the control of, a promoter which can direct the expression in the plant cell. This is preferably accomplished by inserting a chimeric gene comprising these elements in the plant cell genome, particularly in the nuclear or plastid (e. g. chloroplast) genome.

The promoter, which may be operably linked to SEQ ID NO: 3, or variant or fragment thereof, may for example be a constitutively active promoter, such as: the strong constitutive 35S promoters or enhanced 35S promoters (the “35S promoters”) of the cauliflower mosaic virus (CaMV) of isolates CM 1841 (Gardner et al., 1981), CabbB-S (Franck et al., 1980) and CabbB-JI (Hull and Howell, 1987); the 35S promoter described by Odell et al. (1985) or in U.S. Pat. No. 5,164,316, promoters from the ubiquitin family (e.g. the maize ubiquitin promoter of Christensen et al., 1992; EP 0342926; see also Cornejo et al., 1993), the gos2 promoter (de Pater et al., 1992), the emu promoter (Last et al., 1990), Arabidopsis actin promoters such as the promoter described by An et al. (1996), rice actin promoters such as the promoter described by Zhang et al. (1991) and the promoter described in U.S. Pat. No. 5,641,876 or the rice actin 2 promoter as described in WO070067; promoters of the Cassava vein mosaic virus (WO97/48819, Verdaguer et al. 1998), the pPLEX series of promoters from Subterranean Clover Stunt Virus (WO96/06932, particularly the S7 promoter), a alcohol dehydrogenase promoter, e.g., pAdh1S (GenBank accession numbers X04049, X00581), and the TR1′ promoter and the TR2′ promoter (the “TR1′ promoter” and “TR2′ promoter”, respectively) which drive the expression of the 1′ and 2′ genes, respectively, of the T-DNA (Velten et al., 1984), the Figwort Mosaic Virus promoter described in U.S. Pat. No. 6,051,753 and in EP426641, histone gene promoters, such as the Ph4a748 promoter from Arabidopsis (PMB 8: 179-191), or others.

Alternatively, a promoter can be utilized, which is not constitutive but rather is specific for one or more tissues or organs of the plant (tissue preferred/tissue specific, including developmentally regulated promoters), for example an egg cell specific promoter, whereby the protein of the invention is expressed only or preferentially in cells of the specific tissue(s) or organ(s) and/or only during a certain developmental stage.

As the constitutive production of the protein of the invention may have a high cost on fitness of the plants, it is in one embodiment preferred to use a promoter whose activity is inducible. Examples of inducible promoters are wound-inducible promoters, such as the MPI promoter described by Cordera et al. (1994), which is induced by wounding (such as caused by insect or physical wounding), or the COMPTII promoter (WO0056897) or the PR1 promoter described in U.S. Pat. No. 6,031,151. Alternatively the promoter may be inducible by a chemical, such as dexamethasone as described by Aoyama and Chua (1997) and in U.S. Pat. No. 6,063,985 or by tetracycline (TOPFREE or TOP 10 promoter, see Gatz, 1997 and Love et al., 2000).

The word “inducible” does not necessarily require that the promoter is completely inactive in the absence of the inducer stimulus. A low level non-specific activity may be present, as long as this does not result in severe yield or quality penalty of the plants. Inducible, thus, preferably refers to an increase in activity of the promoter, resulting in an increase in transcription of the downstream coding region encoding the protein of the invention following contact with the inducer.

In one embodiment the promoter of a native parthenogenesis gene is used. For example, the promoter of the Taraxacum Par or par allele may be isolated and operably linked to the coding region encoding the protein according to the invention. In an embodiment, said promoter (the upstream transcription regulatory region, e.g. within about 2000 bp upstream of the translation start codon and/or transcription start codon) can be isolated from apomictic plants and/or other plants using known methods, such as TAIL-PCR (Liu et al., 1995; Liu et al., 2005), Linker-PCR, or Inverse PCR (IPCR).

In one embodiment, a promoter of a native parthenogenesis gene is used, or a promoter derived therefrom. For example, a promoters derived from SEQ ID NO: 2, or a variant or fragment thereof, may be used. Preferably, said promoter is a promoter comprising a nucleic acid insert, preferably a double-stranded DNA insert, wherein said insert has a length of between 50 and 2000 bp, between 100 and 1900 bp, between 200 and 1800 bp, between 300 and 1700 bp, between 400 and 1600 bp, between 500 and 1500 bp, between 600 and 1400 bp, between 1000 and 1400, between 1200 and 1400, or between 1300 and 1400 bp. Even more preferably, said insert has a length of about 1300 bp. Preferably, the insert is associated with, and optionally is functional in the parthenogenesis phenotype as defined herein. Preferably, said insert is localized within the promoter sequence that is localized directly upstream (3′) of the sequence encoding the PAR protein, preferably such that the distance between the 3′ end of said insert and the start codon of the sequence encoding the PAR protein is between 50-200 bp, preferably about 50, 60, 70, 80, 90, 100, 110, 120, 130, 140, 150, 160, 170, 180, 190 or 200 bp, most preferably about 102 bp. Preferably, said insert is localized such that the 3′ end nucleotide of the insert is at a position that is homologous to the position of nucleotide 1798 of SEQ ID NO: 2 and/or of nucleotide 1798 of SEQ ID NO: 5. Preferably, said insert is devoid of an open reading frame. Even more preferably said insert is a Miniature Inverted-Repeat Transposable Elements (MITE) or MITE-like sequence, wherein said MITE or MITE-like sequence is a non-autonomous element characterized that contains an internal sequence devoid of an open reading frame, that is flanked by terminal inverted repeats (TIRs) which in turn are flanked by small direct repeats (target site duplications). For a further description of MITE, TIR and sequences, referred is to Guo et al, Scientific Reports. 2017 Jun. 1; 7(1):2634 which is incorporated herein by reference. Said insert, preferably said MITE or MITE-like sequence, may have at least about 50%, 60%, 70%, 80%, 90%, 95%, 98%, 99% or more identity to SEQ ID NO: 60. Preferably, said insert is associated with, and optionally is functional in the parthenogenesis phenotype as defined herein. The promoter may have the nucleotide sequence of SEQ ID NO: 2. Also sequences which are longer than the sequences mentioned herein may be used. A region up to about 2000 bp upstream of the translation start codon of a coding region may comprise transcription regulatory elements (i.e. promoter). Thus, in one embodiment the nucleotide sequence 2000 bp, 1500 bp, 1000 bp, 800 bp, 500 bp, 300 bp or less upstream of the translation start codon of a sequence encoding the protein of the invention is isolated, promoter activity may be tested and, if functional, the sequence may be operably linked to a sequence encoding the protein of the invention as taught herein. Promoter activity of whole sequences and fragments thereof can be tested by e.g. deletion analysis, whereby 5′ and/or 3′ parts are deleted and the promoter activity is tested using known methods (e.g. operably linking the promoter or fragment to a reporter gene).

A coding sequence as taught herein is preferably inserted into the plant genome so that the coding sequence is upstream (i.e. 5) of suitable 3′ end non-translated region (“3′ end” or 3′UTR). Suitable 3′ ends include those of the CaMV 35S gene (“3′ 35S”), the nopaline synthase gene (“3′ nos”) (Depicker et al., 1982), the octopine synthase gene (“3′ ocs”) (Gielen et al., 1984) and the T-DNA gene 7 (“3′ gene 7”) (Velten and Schell, 1985), which act as 3′-untranslated DNA sequences in transformed plant cells, and others. In one embodiment, a 3′UTR of a native parthenogenesis gene is used, or a 3′UTR derived therefrom. For example, any 3′UTR derived from SEQ ID NO: 4, or a variant or fragment thereof, may be used. The 3′UTR may have the nucleotide sequence of SEQ ID NO: 4.

In an embodiment, a promoter having a nucleotide sequence of SEQ ID NO: 2, or variant and/or fragment hereof, may be operably linked to nucleic acid encoding the protein of the invention, preferably the nucleotide sequence encoding the protein is capable of inducing parthenogenesis as taught herein, more preferably having the amino acid sequence of SEQ ID NO: 1, or variant and/or fragment thereof. Preferably, said promoter and coding sequence are further operably linked to a 3′UTR of SEQ ID NO: 4, or variant and/or fragment thereof.

Introduction of the T-DNA vector into Agrobacterium can be carried out using known methods, such as electroporation or triparental mating.

A coding sequence as taught herein, can optionally be inserted in the plant genome as a hybrid gene sequence whereby the coding sequence is linked in-frame to a (U.S. Pat. No. 5,254,799; Vaeck et al., 1987) gene encoding a selectable or scorable marker, such as for example the neo (or nptII) gene (EP0242236) encoding kanamycin resistance, so that the plant expresses a fusion protein which is easily detectable.

All or part of a sequence encoding the protein of the invention can also be used to transform microorganisms, such as bacteria (e.g. Escherichia coli, Pseudomonas, Agrobacterium, Bacillus, etc.), fungi, or algae or insects, or to make recombinant viruses. This is in particular suitable for production and subsequent purification of the protein, preferably isolated protein. Transformation of bacteria, with all or part of the coding sequence as taught herein, incorporated in a suitable cloning vehicle, can be carried out in a conventional manner, preferably using conventional electroporation techniques as described in Maillon et al. (1989) and WO 90/06999. For expression in prokaryotic host cell, the codon usage of the nucleotide sequence may be optimized accordingly (as described for plants herein). Intron sequences should be removed and other adaptations for optimal expression may be made as known. Such prokaryotic host cell comprising the nucleic acid and/or expressing the protein of the invention are encompassed by the present invention. Such host cells may be used to produce a protein and/or nucleic acid of the invention.

The DNA sequence of the nucleic acid of the invention can be further changed in a translationally neutral manner, to modify possibly inhibiting DNA sequences present in the gene part and/or by introducing changes to the codon usage, e. g., adapting the codon usage to that most preferred by plants, preferably the specific relevant plant genus, e.g. as described herein as host plants.

In accordance with one embodiment of this invention, the protein of the invention is targeted to intracellular organelles such as plastids, preferably chloroplasts, mitochondria, or are secreted from the cell, potentially optimizing protein stability and/or expression. Similarly, the protein may be targeted to vacuoles. For this purpose, in one embodiment of this invention, the chimeric gene of the invention comprises a coding region encoding a signal or target peptide, linked to the region encoding the protein of the invention. Particularly preferred peptides to be included in the proteins of this invention are the transit peptides for chloroplast or other plastid targeting, especially duplicated transit peptide regions from plant genes whose gene product is targeted to the plastids, the optimized transit peptide of Capellades et al. (U.S. Pat. No. 5,635,618), the transit peptide of ferredoxin-NADP+oxidoreductase from spinach (Oelmuller et al., 1993), the transit peptide described in Wong et al. (1992) and the targeting peptides in published PCT patent application WO 00/26371. Also preferred are peptides signalling secretion of a protein linked to such peptide outside the cell, such as the secretion signal of the potato proteinase inhibitor II (Keil et al., 1986), the secretion signal of the alpha-amylase 3 gene of rice (Sutliff et al., 1991) and the secretion signal of tobacco PR1 protein (Cornelissen et al., 1986). Particularly useful signal peptides in accordance with the invention include the chloroplast transit peptide (e.g. Van Den Broeck et al., 1985), or the optimized chloroplast transit peptide of U.S. Pat. Nos. 5,510,471 and 5,635,618 causing transport of the protein to the chloroplasts, a secretory signal peptide or a peptide targeting the protein to other plastids, mitochondria, the ER, or another organelle. Signal sequences for targeting to intracellular organelles or for secretion outside the plant cell or to the cell wall are found in naturally targeted or secreted proteins, preferably those described by Klösgen et al. (1989), Klösgen and Weil (1991), Neuhaus & Rogers (1998), Bih et al. (1999), Morris et al. (1999), Hesse et al. (1989), Tavladoraki et al. (1998), Terashima et al. (1999), Park et al. (1997), Shcherban et al. (1995).

In one embodiment, the protein of the invention as taught herein is co-expressed with other proteins which control, preferably enhance or induce, parthenogenesis, apomeiosis or apomixis in a single host, optionally under control of different promoters. Such other gene may be the gene for conferring apomeiosis, such as diplospory e.g. as described in WO2017/039452 A1, which is incorporated herein by reference.

In another embodiment, the protein of the invention is introgressed in germplasm that preferably comprises other genes of interest, such as the gene for conferring apomeiosis (e.g. the gene for diplospory). Via crossing and selection, hybrids are produced wherein several genes of interest may be stacked.

A co-expressing host plant is easily obtained by transforming a plant already expressing a protein of this invention, or by crossing plants transformed with different nucleic acids of this invention. It is understood that the different proteins can be expressed in the same plant, or each can be expressed in a single plant and then combined in the same plant by crossing the single plants with one another. For example, in hybrid seed production, each parent plant can express each of the proteins desired to be co-expressed. Upon crossing the parent plants to produce hybrids, both proteins are combined in the hybrid plant. Such hybrid or offspring thereof comprising the both genes and/or expressing both proteins is encompassed by the present invention.

Preferably, for selection purposes but also for weed control options, the transgenic plants of the invention are also transformed with a DNA encoding a protein conferring resistance to herbicide, such as a broad-spectrum herbicide, for example herbicides based on glufosinate ammonium as active ingredient (e.g. Liberty® or BASTA; resistance is conferred by the PAT or bar gene; see EP 0 242 236 and EP 0 242 246) or glyphosate (e.g. RoundUp®; resistance is conferred by EPSPS genes, see e.g. EP0 508 909 and EP 0 507 698). Using herbicide resistance genes (or other genes conferring a desired phenotype) as selectable marker further has the advantage that the introduction of antibiotic resistance genes can be avoided.

Alternatively or in addition, other selectable marker genes may be used, such as antibiotic resistance genes. As it is generally not accepted to retain antibiotic resistance genes in the transformed host plants, these genes can be removed again following selection of the transformants. Different technologies exist for removal of transgenes. One method to achieve removal is by flanking the transgene with lox sites and, following selection, crossing the transformed plant with a CRE recombinase-expressing plant (see e.g. EP506763B1). Site specific recombination results in excision of the marker gene. Another site specific recombination system is the FLP/FRT system described in EP686191 and U.S. Pat. No. 5,527,695. Site specific recombination systems such as CRE/LOX and FLP/FRT may also be used for gene stacking purposes. Further, one-component excision systems have been described, see e.g. WO9737012 or WO9500555).

Preferably, the nucleic acid of the invention is used to generate transgenic plant cells, plants, plant seeds, etc. and any derivatives/progeny thereof, with an enhanced parthenogenetic phenotype. A transgenic plant with enhanced parthenogenesis can be generated by transforming a plant host cell with the nucleic acid of the invention preferably encoding the protein having the amino acid sequence of SEQ ID NO: 1 or variant and/or fragment thereof, under the control of a suitable promoter, as described herein, and regenerating a transgenic plant from said cell. Preferably, the transgenic plants of the invention comprise enhanced parthenogenesis compared to the non-transformed or empty vector control. Thus, for example transgenic lettuce plants comprise enhanced parthenogenesis are provided. Thus, a transformed plant expressing the protein according to the invention shows enhanced parthenogenesis if it shows a significant increase in parthenogenesis, as compared to the untransformed or empty-vector transformed control. The enhanced parthenogenesis phenotype can be fine-tuned by expressing a suitable amount of the protein of the invention capable of inducing parthenogenesis at a suitable time and/or location. Such fine-tuning may be done by determining the most appropriate promoter and/or by selecting transgenic “events” which show the desired expression level.

Transformants, hybrids or inbreds expressing desired levels of the protein of the invention and/or comprising the desired, or desired levels of, the nucleic acid of the invention are selected by e.g. analysing copy number (Southern blot analysis), mRNA transcript levels (e.g. RT-PCR using primer pairs capable of amplifying the protein of the invention or flanking primers) or by analysing the presence and level of parthenogenesis protein in various tissues (e.g. SDS-PAGE; ELISA assays, etc). Single copy transformants may be selected, for instance for regulatory reasons, and the sequences flanking the site of insertion of the transgene is analysed, preferably sequenced to characterize the “event”. Transgenic events resulting in high or moderate expression of the protein of the invention are selected for further development until a high performing elite event with a stable transgene is obtained.

Transformants expressing a protein of the invention and/or comprising a nucleic acid of the invention, may also comprise other transgenes, such as other genes conferring disease resistance or conferring tolerance to other biotic and/or abiotic stresses, or conferring diplospory. To obtain such plants with “stacked” transgenes, other transgenes may either be introduced into said transformants, or said transformants may be transformed subsequently with one or more other genes, or alternatively several chimeric genes may be used to transform a plant line or variety. For example, several transgenes may be present on a single vector, or may be present on different vectors which are co-transformed.

In one embodiment the following genes are combined with the nucleic acid of the invention: known disease resistance genes, especially genes conferring enhanced resistance to necrotrophic pathogens, virus resistance genes, insect resistance genes, abiotic stress resistance genes (e.g. drought tolerance, salt tolerance, heat- or cold tolerance, etc.), herbicide resistance genes, and the like. The stacked transformants may thus have an even broader biotic and/or abiotic stress tolerance, to pathogen resistance, insect resistance, nematode resistance, salinity, cold stress, heat stress, water stress, etc. Also, silencing approaches may be combined with expression approaches in a single plant, for instance silencing of a Par allele may be combined with expression of a par allele, or vice versa.

Optionally, the nucleic acid of the invention may be used to repress parthenogenesis, for instance by silencing, knocking down or reducing expression of a parthenogenesis gene on one or more Par alleles in a plant or plant cell. This may be done by modifying the encoding sequence or one or more regulatory sequences (e.g. promoter sequence) of the Par allele(s), present in said plant or plant cell, or by introducing a RNAi targeting transcripts of the Par allele(s). Therefore, the invention also provides for a method for reducing or abolishing parthenogenesis in a plant or plant cell, comprising the steps of:
a) reducing or abolishing expression of a nucleic acid capable of inducing parthenogenesis and/or functional in parthenogenesis as defined herein in one or more plant cells;
b) selecting a plant cell wherein said expression is reduced or abolished; and
c) regenerating a plant from said plant cell.

Said nucleic acid preferably is a nucleic acid comprising or consisting of any one of SEQ ID NOs: 2-5, and variants and/or fragments thereof, and/or a nucleic acid encoding a protein of SEQ ID NO: 1, and/or variant or fragment thereof.

Whole plants, plant parts (e.g. seeds, cells, tissues), and plant products (e.g. fruits) and progeny of any of the transformed plants described herein are encompassed herein and can be identified by the presence of the transgene, for example by PCR analysis using total genomic DNA as template and using PCR primer pairs specific for parthenogenesis gene and/or by using genomic variation analysis such as, but not limited to, Sequence Based Genotyping (SBG) or KeyGene® SNPSelect analysis. Also “event specific” PCR diagnostic methods can be developed, where the PCR primers are based on the plant DNA flanking the inserted transgene, see U.S. Pat. No. 6,563,026. Similarly, event specific AFLP fingerprints or RFLP fingerprints may be developed which identify the transgenic plant or any plant, seed, tissue or cells derived there from.

It is understood that the transgenic plants according to the invention preferably do not show non-desired phenotypes, such as yield reduction, enhanced susceptibility to diseases (especially to necrotrophs) or undesired architectural changes (dwarfing, deformations) etc. and that, if such phenotypes are seen in the primary transformants, these can be removed by conventional methods. Any of the transgenic plants described herein may be heterozygous, homozygous or hemizygous for the transgene.

The invention also pertains to a plant, seed, plant part (e.g. a plant cell) and plant product obtained or obtainable by the method as detailed herein, preferably comprising the protein of the invention, the nucleic acid of the invention and/or the construct of the invention. Preferably said protein, nucleic acid and/or construct are capable of inducing parthenogenesis and/or functional in parthenogenesis, as detailed herein. The plant of the invention preferably is of a species listed herein as suitable host plant. Such method includes introgression of the nucleic acid of the invention from a plant into progeny, and/or transformation of plant cells by a nucleic acid of the invention as transgene, and subsequent regeneration of a plant from said plant cell. Preferably the plant, plant part and/or plant product is not of the species Taraxacum officinale sensu lato, comprising a nucleic acid of the invention, wherein said plant or plant cell preferably is of a species listed herein as suitable host plant, preferably from the family selected from the group consisting of Brassicaceae, Cucurbitaceae, Fabaceae, Gramineae, Solanaceae and Asteraceae (Compositae).

Preferably plant, plant part and/or plant product comprises the nucleic acid of the invention by genetic modification or by introgression, wherein preferably said nucleic acid is integrated in its genome. Preferably said plant, plant part and/or plant product is capable of parthenogenesis and/or functional in parthenogenesis. Even more preferably said plant, plant part and/or plant product is further capable of apomeiosis. The invention provides for seed, plant parts or plant products of a plant or plant cell of the invention.

The invention also pertains to plant parts and plant products derived from the plant of the invention, wherein the plant parts and/or plant products comprise the protein of the invention as defined herein, the nucleic acid of the invention as defined herein and/or the construct of the invention as defined herein, which may be fragments as defined herein that allow for assessing the presence of such protein, nucleic acid or construct in the plant from which the plant part of plant product is derived. Such parts and/or products may be seed or fruit and/or products derived therefrom (e.g. sugars or protein). Such parts, products and/or products derived therefrom may be non-propagating material.

Any plant may be a suitable host, but most preferably the host plant species should be a plant species which would benefit from enhanced or reduced parthenogenesis. Suitable hosts include any plant species. Particularly, cultivars or breeding lines having otherwise good agronomic characteristics are preferred. The skilled person knows how to test whether the nucleic acid and/or protein as taught herein, and/or variants or fragments thereof, can confer the required increase or reduction of parthenogenesis onto the host plant, by generating transgenic plants and assessing parthenogenesis, together with suitable control plants.

Suitable host plants include for example hosts which belong to the Brassicaceae, Cucurbitaceae, Fabaceae, Gramineae, Solanaceae, Asteraceae (Compositae), Rosaceae or Poaceae.

In a preferred embodiment, the host plant may be a plant species selected from the group consisting of the genera Taraxacum, Lactuca, Pisum, Capsicum, Solanum, Cucumis, Zea, Gossypium, Glycine, Tryticum, Oryza and Sorghum.

In a preferred embodiment, the plant, plant part, plant cell or seed as taught herein is from a species selected from the group consisting of the genera Taraxacum, Lactuca, Pisum, Capsicum, Solanum, Cucumis, Zea, Gossypium, Glycine, Triticum, Oryza, Allium, Brassica, Helianthus, Beta, Cichorium, Chrysanthemum, Pennisetum, Secale, Hordeum, Medicago, Phaseolus, Rosa, Lilium, Coffea, Linum, Canabis, Cassava, Daucus, Cucurbita, Citrullus, and Sorghum.

Suitable host plants include for example maize/corn (Zea species), wheat (Triticum species), barley (e.g. Hordeum vulgare), oat (e.g. Avena sativa), sorghum (Sorghum bicolor), rye (Secale cereale), soybean (Glycine spp, e.g. G. max), cotton (Gossypium species, e.g. G. hirsutum, G. barbadense), Brassica spp. (e.g. B. napus, B. juncea, B. oleracea, B. rapa, etc), sunflower (Helianthus annus), safflower, yam, cassava, alfalfa (Medicago sativa), rice (Oryza species, e.g. O. sativa indica cultivar-group or japonica cultivar-group), forage grasses, pearl millet (Pennisetum spp. e.g. P. glaucum), tree species (Pinus, poplar, fir, plantain, etc), tea, coffea, oil palm, coconut, vegetable species, such as pea, zucchini, beans (e.g. Phaseolus species), hot pepper, cucumber, artichoke, asparagus, eggplant, broccoli, garlic, leek, lettuce, onion, radish, turnip, tomato, potato, Brussels sprouts, carrot, cauliflower, chicory, celery, spinach, endive, fennel, beet, fleshy fruit bearing plants (grapes, peaches, plums, strawberry, mango, apple, plum, cherry, apricot, banana, blackberry, blueberry, citrus, kiwi, figs, lemon, lime, nectarines, raspberry, watermelon, orange, grapefruit, etc.), ornamental species (e.g. Rose, Petunia, Chrysanthemum, Lily, Gerbera species), herbs (mint, parsley, basil, thyme, etc.), woody trees (e.g. species of Populus, Salix, Quercus, Eucalyptus), fibre species e.g. flax (Linum usitatissimum) and hemp (Cannabis sativa).

### Marker Assisted Selection and Transfer or Combination of One or More Par Alleles

The nucleic acid of the invention can be used as a genetic marker for marker assisted selection of the Par or par alleles of Taraxacum species and/or of other plant species and for the transfer and/or combination of different or identical Par or par alleles to/in plants of interest and/or to/in plants which can be used to generate intraspecific or interspecific hybrids with the plant in which the Par or par allele (or variant) is found.

Many different marker assays can be developed based on these sequences. The development of a marker assay generally involves the identification of polymorphisms between Par and par alleles, so that the polymorphism is a genetic marker which “marks” a specific allele. The polymorphism(s) is/are then used in a marker assay. For example the sequence of the Par allele as taught herein, is correlated with the presence or enhancement of parthenogenesis. This is for example done by screening parthenogenetic plant material and/or non-parthenogenetic plant material for (part of) the nucleotide sequence of the Par or par allele as taught herein in order to correlate specific alleles with parthenogenesis or non-parthenogenesis. Thus, PCR primers or probes may be generated which detect such nucleotide sequence in a sample (e.g. an RNA, cDNA or genomic DNA sample) obtained from (non-)parthenogenetic plant material. The sequences or parts thereof are compared and polymorphic markers are identified which correlate with parthenogenesis. The polymorphic marker, such as a SNP marker linked to a Par or par allele, can then be developed into a rapid molecular assay for screening plant material for the presence or absence of the parthenogenesis allele. Thus, the presence or absence of these “genetic markers” is indicative of the presence of the Par or par allele linked thereto and one can replace the detection of the Par or par allele with the detection of the genetic marker.

Preferably, easy and fast marker assays are used, which enable the rapid detection of the Par or par allele, or allele combinations, in samples (e.g. DNA samples). Thus, in one embodiment, the use of a nucleic acid of the invention, in a molecular assay for determining the presence or absence of a Par or par allele in the sample, and/or for determining homozygosity or heterozygosity of this allele, is provided herein.

Such an assay may for example involve the following steps:
(a) providing parthenogenetic and non-parthenogenetic plant material and/or nucleic acid samples thereof;
(b) determining the nucleotide sequence of all or part of the nucleic acid of the invention in said material of (a).

In one aspect, PCR primers and/or probes, molecular markers and kits for detecting the nucleic acid of the invention, or related or derived RNA sequence (such as transcripts), are provided. Degenerate or specific PCR primer pairs to amplify the nucleic acid of the invention from samples can be synthesized based on the nucleotidesequences as taught herein, or variants thereof, as known in the art (see Dieffenbach and Dveksler, 1995; and McPherson at al., 2000). For example, any stretch of 9, 10, 11, 12, 13, 14, 15, 16, 18 or more contiguous nucleotides of this sequence (or the complement strand) may be used as primer or probe.

Likewise, DNA fragments comprising sequences of the Par or par allele as taught herein, or complements thereof, can be used as hybridization probes. A detection kit as provided herein may comprise either Par (allele-) specific primers and/or Par (allele-) specific probes, and an associated protocol to use the primers or probe to detect the nucleic acid of the invention in a sample. Such a detection kit may, for example, be used to determine, whether a plant has been transformed with the nucleic acid of the invention, or to screen Taraxacum germplasm and/or other plant species germplasm for the presence of Par alleles and optionally zygosity determination.

In one embodiment therefore a method of detecting the presence or absence of a nucleotide sequence encoding a protein of the invention in a plant tissue, e.g. in Taraxacum tissue, or a nucleic acid sample thereof is provided. The method may comprises:
a) obtaining a plant tissue sample from one or more plants, or nucleic acid sample thereof,
b) analyzing the nucleic acid sample using a molecular marker assay for the presence or absence of one or more markers linked to a Par allele, wherein the marker assay detects the presence of a nucleic acid of the invention that is associated with parthenogenesis, and optionally
c) selecting the plant comprising one or more of said markers for further use.

Alternatively or in addition, the method may comprises:
a) obtaining a plant tissue sample from one or more plants, or nucleic acid sample thereof,
b) analyzing the nucleic acid sample using a molecular marker assay for the presence or absence of one or more markers linked to a par allele, wherein the marker assay detects the presence of a nucleic acid of the invention that is associated with non-parthenogenesis, and optionally
c) selecting the plant comprising one or more of said markers for further use.
   Preferably the one or more plants use in any of these methods is a plant that is suitable as a host plant as further defined herein.

### Applications of Parthenogenesis

A nucleic acid and/or protein of the invention may be used for screening (e.g. for one or more parthenogenesis locus in a plant or plant cell), genotyping, conferring parthenogenesis, for conferring apomixis for increasing ploidy and/or for producing a double haploid. Preferably said use is in plant biotechnology and/or breeding, i.e. in/on plant or plant cells.

Parthenogenesis is an element of apomixis and a gene for parthenogenesis could be used in combination with a gene for apomeiosis (e.g. diplospory) to generate apomixes, preferably to use it for the applications listed herein. These genes can be introduced into sexual crops by transformation, introgression or by modifying endogenous suitable genes thereby converting them in apomeiotic (or diplosporous) genes. Knowledge of the structure and function of the apomixis genes can also be used to modify endogenous sexual reproduction genes in such a way that they become apomixis genes. The preferred use would be to bring the apomixis genes under a inducible promoter such that apomixis can be switched off when sexual reproduction generates new genotypes and switched on when apomixis is needed to propagate the elite genotypes.

The nucleic acid or its derived product can be used as a component of apomixis. Both apomeiosis and parthenogenesis are required for functional gametophytic apomixis. Apomeiosis can be achieved by a combination of mutations affecting meiosis (Crismani et al., 2013), with the outcome of chromosomal non-reduction in megaspores, i.e., mitosis rather than meiosis. Somatic cells that assume a gametophytic fate through epigenetic alterations (Grimanelli, 2012) also result in unreduced spore-like cells that potentially can give rise to unreduced gametes (egg cells). In another embodiment, apomeiosis is achieved by transgenic or non-transgenic expression of a natural apomeiosis gene. By whatever means unreduced egg cells are formed, proper temporal and spatial expression of a nucleic acid of the invention capable of inducing parthenogenesis can induce the egg cells to behave as zygotes and divide in the absence of fertilization.

A parthenogenesis gene could be used in entirely new ways, e.g. not directly as tool in apomixis. For example whereas in apomixis both parthenogenesis and apomeiosis are combined in a single plant, the use of apomeiosis in one generation and the use of parthenogenesis in the next generation would link sexual gene pools of a crop at the diploid and at the polyploid level, by going up in ploidy level by apomeiosis and going down in ploidy level by parthenogenesis. This is very useful because polyploid populations may be better for mutation induction because they can tolerate more mutations. Polyploid plants can also be more vigorous. However diploid populations are better for selection and diploid crosses are better for genetic mapping, the construction of BAC libraries etc. Parthenogenesis in polyploids may generate haploids which can be crossed with diploids. Diplospory in diploids generates unreduced 2n egg cells which can be fertilized by pollen from polyploids to produce polyploid offspring. Thus, an alternation of apomeiosis and parthenogenesis in different breeding generations links the diploid and the polyploid gene pools.

Another use of the nucleic acid its derived product (transcript or encoded protein) without apomeiosis, is the production of haploid offspring, which could be used for the production of haploids and by genome doubling of doubled haploids (DHs) (e.g. spontaneous genome doubling, colchicine, sodium azide or other chemicals). Doubled haploids can be used as parents to produce sexual F1 hybrids. Doubled haploids is the fastest methods to make plants homozygous. With doubled haploids plants can be made homozygous, whereas with the second fastest method, selfing, it takes 5-7 generations to reach a sufficiently high level of homozygosity in diploid plants. There are several methods to produce doubled haploids. In some plant species haploids can be generated by microspore culture. Other methods are the production of haploid embryos (gynogenesis) by pollination with irradiated pollen (melon), or the pollination with specific pollinator stocks (maize, potato). These methods have their limitations, such as costs, recalcitrance of genotypes, labour intensity etc. In some crops no methods for haploid production exist (e.g. tomato). With the dominant allele of the parthenogenesis gene the frequency of gynogenesis could be significantly increased, reducing the costs of haploid production.

The following non-limiting Examples illustrate the different embodiments of the invention. Unless stated otherwise in the Examples, all recombinant DNA techniques are carried out according to standard protocols as described in Sambrook et al. (1989), and Sambrook and Russell (2001); and in Volumes 1 and 2 of Ausubel et al. (1994). Standard materials and methods for plant molecular work are described in Plant Molecular Biology Labfax (1993) by R. D. D. Croy, jointly published by BIOS Scientific Publications Ltd (UK) and Blackwell Scientific Publications, UK.

TABLE 1
Overview of SEQ ID NOs used herein.

| SEQ ID NO | name |
|---|---|
| 1 | Par allele protein Taraxacum officinale |
| 2 | Par allele promoter Taraxacum officinale |
| 3 | Par allele coding sequence Taraxacum officinale |
| 4 | Par allele 3′UTR Taraxacum officinale |
| 5 | Par allele gene Taraxacum officinale |
| 6 | par allele-1 protein Taraxacum officinale |
| 7 | par allele-1 promoter Taraxacum officinale |
| 8 | par allele-1 coding sequence Taraxacum officinale |
| 9 | par allele-1 3′UTR gene Taraxacum officinale |
| 10 | par allele-1 gene Taraxacum officinale |
| 11 | par allele-2 protein Taraxacum officinale |
| 12 | par allele-2 promoter Taraxacum officinale |
| 13 | par allele-2 coding sequence Taraxacum officinale |
| 14 | par allele-2 3′UTR gene Taraxacum officinale |
| 15 | par allele-2 gene Taraxacum officinale |
| 16 | parsley ubiquitin promoter sequence |
| 17 | Cas9 gene |
| 18 | tomato U6 promoter |
| 19 | gene specific part of guide RNA-1 for Par allele |
| 20 | gene specific part of guide RNA-2 for Par allele |
| 21 | Helianthus annuus _XR_002563155.1 |
| 22 | Lactuca sativa _PLY80414.1 |
| 23 | nucleotides 325-360 of the Par allele (wild type) |
| 24 | mutated sequence of nucleotides 325-360 of the Par allele (1 bp insertion) |
| 25 | mutated sequence of nucleotides 325-360 of the Par allele (1 bp insertion) |
| 26 | mutated sequence of nucleotides 325-360 of the Par allele (1 bp deletion) |
| 27 | mutated sequence of nucleotides 325-360 of the Par allele (3 bp deletion) |
| 28 | encoded amino acid sequence of SEQ ID NO: 24 |
| 29 | encoded amino acid sequence of SEQ ID NO: 25 |
| 30 | encoded amino acid sequence of SEQ ID NO: 26 |
| 31 | encoded amino acid sequence of SEQ ID NO: 27 |
| 32 | encoded amino acid sequence of SEQ ID NO: 23 |
| 33 | DIP forward primer (DIP_F) |
| 34 | DIP reversed primer (DIP_R) |
| 35 | PAR forward primer (PAR_F) |
| 36 | PAR reversed primer (PAR_R) |
| 37 | CXXCXXXXXXX[K/R]AXXGHX[R/N]XH K2-2 zinc finger domain |
| 38 | CXXCXXXXXXX[X]XXXGHXRXH zinc finger domain consensus sequence |
| 39 | Cichorium endivia PAR protein |
| 40 | Hieracium praealtum of aurantiacum PAR protein |
| 41 | Senecio cambrensis PAR protein |
| 42 | Hevea brasiliensis PAR protein |
| 43 | Cucurbita moschata PAR protein |
| 44 | Eutrema salsugineum PAR protein |
| 45 | Arachis ipaensis PAR protein |
| 46 | Cajanus cajan PAR protein |
| 47 | Brassica_rapa PAR protein |
| 48 | Lagenaria siceraria PAR protein |
| 49 | Arachis ipaensis PAR protein |
| 50 | Prunus_persica PAR protein |
| 51 | Glycine max PAR protein |
| 52 | Glycine max PAR protein |
| 53 | Glycine max PAR protein |
| 54 | Glycine max PAR protein |
| 55 | Cicer arietinum_fabales PAR protein |
| 56 | Cicer arietinum PAR protein |
| 57 | Cicer arietinum PAR protein |
| 58 | EAR motif |
| 59 | EAR motif |
| 60 | Tar-MITE insert |

TABLE 2
Effect of T-DNA constructs encoding either Cas9/gRNA-1 or Cas9/gRNA-2 on seed
phenotype, the Par allele, more in particular on the stretch of nucleotides 325-
360 of the Par allele (SEQ ID NO: 23) and the encoded amino acid stretches.

| | | | Type of | Resulting |
|---|---|---|---|---|
| | | | mutation (resulting | encoded amino |
| Plant ID | gRNA | Phenotype | nucleotide sequence) | acid sequence |
| pKG10821-1 | gRNA-1 | light grey seed | 1 bp insertion | SEQ ID NO: 28 |
| | | | (SEQ. ID NO: 24) | |
| pKG10821-4 | gRNA-1 | light grey seed | 1 bp insertion | SEQ ID NO: 29 |
| | | | (SEQ ID NO: 25) | |
| pKG10821-5 | gRNA-1 | light grey seed | 1 bp deletion | SEQ ID NO: 30 |
| | | | (SEQ ID NO: 26) | |
| pKG10821-6 | gRNA-1 | light grey seed | 3 bp deletion | SEQ ID NO: 31 |
| | | | (SEQ ID NO: 27) | |
| pKG10821-7 | gRNA-1 | normal dark seed | no mutation | SEQ ID NO: 32 |
| | | | (SEQ ID NO: 23) | |
| pKG10821-8 | gRNA-1 | normal dark seed | no mutation | SEQ ID NO: 32 |
| | | | (SEQ ID NO: 23) | |
| pKG10822-2 | gRNA-1 | normal dark seed | no mutation | SEQ ID NO: 32 |
| | | | (SEQ ID NO: 23) | |
| pKG10822-8 | gRNA-1 | normal dark seed | no mutation | SEQ ID NO: 32 |
| | | | (SEQ ID NO: 23) | |
| pKG10672-1 | gRNA-2 | light grey seed | not sequenced | not sequenced |
| pKG10672-2 | gRNA-2 | light grey seed | not sequenced | not sequenced |

### FIGURE LEGENDS

FIG. 1: Multiple sequence alignment of the coding sequences (nucleotides 325-360 of the Par allele coding sequence), and the encoded amino acids, of amplicons from the control plant showing the wild type sequence (SEQ ID NO: 23) and from transgenic plants comprising the vector encoding the Cas9/RNA-1 complex showing the modified sequences (SEQ ID NO: 24-27). The gene specific part of guide RNA-1 is indicated with a box. Modifications are indicated in bold and underlined. The wild type sequence comprises spacing (-) for alignment reasons.

FIG. 2: Germination experiment. Top row; A68 control, normal viable black seeds that germinate. Middle row; non-viable, light grey, not-germinating seeds of plant pKG10821-6 having a 3 bp deletion in gene 164. Bottom row; all tetraploid, germinating and viable offspring of plant pKG10821-6 pollinated with FCH72 haploid pollen. Seeds on each petridish are derived from a single seed head.

FIG. 3: Example of a cleared ovule with an embryo at 75 hours post emasculation of transgenic lettuce line harboring the Par allele gene of Taraxacum officinale driven by the EC1.1 promoter of Arabidopsis thaliana. In case such embryo was found the embryo was taken along in the sum of observation as shown in table 3.

FIG. 4: Example of polyembryony in a cleared ovule at 75 hours post emasculation of transgenic lettuce line harboring the Par allele gene of Taraxacum officinale driven by the EC1.1 promoter of Arabidopsis thaliana. Each asterisk marks an embryo.

FIG. 5. Analysis of Par gene expression in APO, PAR and SEX plants.

### EXAMPLES

### Example 1

### Material and Methods

### Plant Material

Wild type apomictic triploid Taraxacum officinale A68 and sexual diploid Taraxacum officinale FCH72.

### DNA Construct

A binary vector was constructed with the following components encoded on the T-DNA region; a parsley ubiquitin promoter (SEQ ID NO: 16) driving a Cas9 gene (SEQ ID NO: 17) with a 35S terminator, and a tomato U6 promotor (SEQ ID NO: 18, Nekrasov et al, 2013) driving a guide RNA-1 (having a target specific sequence of SEQ ID NO: 19) with a TTTTTT terminator sequence and glufosinate resistance gene for selection. A similar binary vector was constructed wherein the sequence of guide RNA-1 is replaced with a sequence of a guide RNA-2 (having a target specific sequence of SEQ ID NO: 20). Suitable technologies to generate such a binary vector are Gateway®, Golden Gate or Gibson Assembly@ (for an example, see Ma et al., 2015). A vector encoding 35S-GUS on the T-DNA region as used as a control construct.

### Plant Transformation Method

Agrobacterium transformation was performed according to a modified version of the protocol of Oscarsson (Oscarsson, Lotta. “Production of rubber from dandelion—a proof of concept fora new method of cultivation.” 2015). Starting material for plant transformation were Taraxacum officinale A68 explants obtained from subcultured in vitro propagated seed derived plants grown on half strength MS20 medium with 0.8% agar. Overnight cultures of 50 ml in LB medium of Agrobacterium tumefaciens (Rhizobium radiobacter) such as strain C58C1 with the binary vector were used in a 10× dilution (re-suspended and diluted in liquid MS20) for co-cultivation. Explants were cut into pieces of approximately 0.5 cm² and were co-cultivated for 2-3 days. Next, explants were moved to callus inducing medium (CIM; 20 g I-1 sucrose, 4.4 g I-1 MS with micro- and macro nutrients, 8 g I-1 agar, 1 mg I-1 BAP, 0.2 mg I-1 IAA, 3 mg I-1 glufosinate for plant selection, 100 mg I-1 Vancomycine and 100 mg I-1 cefotaxime, pH 5.8). Explants were transferred weekly to fresh CIM. When callus appeared it was transferred to shoot inducing medium (SIM; 20 g I-1 sucrose, 4.4 g I-1 MS with micro- and macro nutrients, 8 g I-1 agar, 2 mg I-1 zeatin, 0.1 mg I-1 IAA, 0.05 mg I-1 GA3, 3 mg I-1 glufosinate for plant selection, 100 mg I-1 Vancomycine and 100 mg I-1 cefotaxime, pH 5.8). Last, formed shoots of a few cm in diameter were rooted in rooting medium (RM; 20 g I-1 sucrose, 2.2 g I-1 MS with micro- and macro nutrients, 8 g I-1 agar, 100 mg I-1 Vancomycine and 100 mg I-1 cefotaxime, pH 5.8). Rooted shoots were transferred to the greenhouse in soil in pots.

### Results

Rooted plants obtained from the Agrobacterium transformation were genotyped for presence of the respective T-DNA encoding the Cas9 and guide RNA-1 or guide RNA-2 in the plant genome by PCR. Plants that were positive for this test (indicated herein as transgenic plants) were grown until seed setting. Individual transgenic plants derived from individual calli comprising any one of these constructs had normal viable dark black grey seeds and some of such plants had aberrant light grey seeds (see Table 2). These light grey seeds were found to be empty, lacking embryos and were found to be non-viable and did not germinate. Control plants (negative for the T-DNA or transformed with a 35S-GUS control construct) never had similar aberrant light grey seeds and control plants all had normal seed heads with fertile black grey seeds. Next, all transgenic plants were genotyped by amplicon sequencing of the guide RNA-1 targeted genomic DNA region on the Illumina MiSeq System. It was found that all transgenic plants that showed the aberrant light grey seeds had small deletions or insertions in the parthenogenesis gene, more in particular within the stretch of DNA targeted by gRNA-1. A68 is a triploid plant. The sequences of this gene on the other two alleles were identified and are represented herein by SEQ ID NO: 10 and 15. The sequences of these two alleles lack the PAM sequence required for the Cas9/guide RNA to induce a DSB.

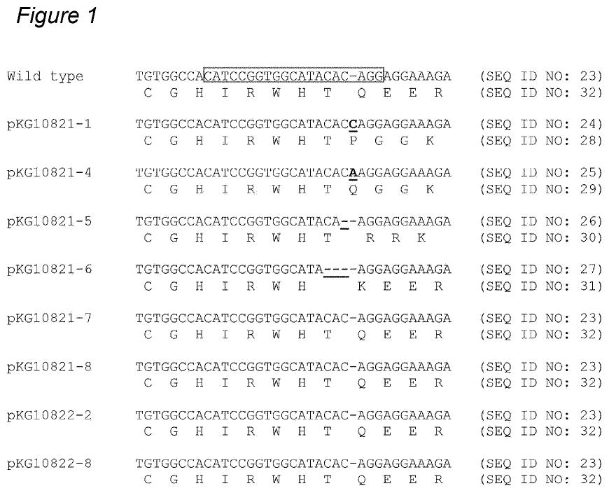

None of the transgenic plants that had normal black seeds had a change in the sequence of the gene. Table 2 summarizes the observed small deletions or insertions and the effect on the translation of the coding sequence to the protein sequence and FIG. 1 shows a multiple sequence alignment of the amplicons.

The seed setting observed for the transgenic plants having a small deletion in the gene of SEQ ID NO: 5 was interpreted as an indication for the loss of apomictic phenotype (denominated herein as Loss-of-Apomixis or LoA), moreover for the loss of parthenogenetic phenotype (Loss-of-Parthenogenesis or LoP). Apomictic plants always carry the dominant Par-allele.

High seed set of triploid Taraxacum, in the absence of cross pollination is a clear indication for apomixis. Selfing can be excluded as an alternative explanation, because due to an unbalanced triploid male and female meiosis, sexually produced egg cells and pollen grains will have a very low fertility. Deletion of the Par-allele results into the LoP and therefore into the LoA. However, LoA can also be caused by the disturbance of other developmental processes. LoP plants are thus a subset of LoA plants and further tests are necessary to identify the observed phenotypes as LoP deletion phenotypes.

In order to further investigate the nature of the observed light grey seed phenotype, crosses were made. LoP in triploid transgenic plants was detected by cross pollinating the triploid transgenic A68 plants with haploid pollen from sexual FCH72 diploid plants. Seeds of these crosses were collected, sown and the ploidy level of the offspring was measured with flow cytometry. Uniformly tetraploid offspring was found, which showed that the LoA plant was diplosporous and capable of seed reproduction, but lacked parthenogenesis.

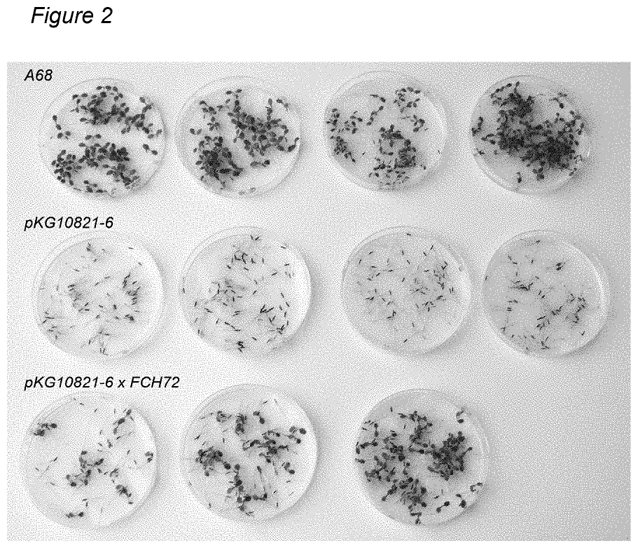

As a control, seeds of apomictic triploid A68 plants were sown and these were all found to be triploid. In the same sowing, seeds from the various plants carrying the T-DNA with guide RNA-1 showing the light grey phenotype were taken along and these seeds were never found to germinate (FIG. 2). A similar germination test result after a cross with FHC72 is anticipated for plants carrying the T-DNA with guide RNA-2 and showing the empty seeds phenotype (germination experiment was not performed). Altogether it was concluded that Taraxacum officinale A86 carries a dominant Par allele having the sequence of SEQ ID NO: 5 that is essential for parthenogenesis, and two recessive sexual alleles having the sequence of SEQ ID NO; 10 and 15, respectively.

### Example 2

A gene essential for parthenogenesis can be used to transfer the parthenogenesis trait to a plant without apomixis or without parthenogenesis. Either the gene or the coding sequence of the gene having SEQ ID NO: 5 or a homologous gene can be used to achieve this. A binary vector is prepared with a T-DNA with at least the gene of SEQ ID NO: 5 or a homologous gene, driven by its native promoter or a female gamete specific promoter. This gene construct is transformed by Agrobacterium mediated transformation to a plant without parthenogenesis, for example lettuce or arabidopsis. Plants positively tested for presence of the transgene are evaluated for occurrence of parthenogenesis. As the trait is dominant, testing is performed on the primary transformed plants (T0). Parthenogenesis can be detected in non-apomictic plants microscopically by Nomarski Differential Interference Microscopy (DIC) of ovules cleared with methyl salicylate (Van Baarlen et al. 2002). In the absence of cross or self-fertilization, parthenogenetic egg cells develop into embryos. On plants harboring the above-mentioned T-DNA at least a few of such embryos are found.

### Plant Material

For this experiment, wild type lettuce: Iceberg type, Legacy, Takii Japan and Red Romaine type, Baker Creek Heirloom Seeds was used.

### DNA Construct

A binary vector was constructed with the following components encoded on the T-DNA region; a EC1.1 promoter of Arabidopsis thaliana (as in Sprunk et al. 2012) driving expression of the Par allele CDS sequence of Taraxacum officinale (SEQ ID NO: 3) followed by the first 250 bases of the 3′UTR (the first 250 bases of SEQ ID NO: 4), followed by a 35S terminator and a neomycin phosphotransferase gene (nptII) for selection. Suitable technologies to generate such a binary vector are Gateway®, Golden Gate or Gibson Assembly@ (for an example, see Ma et al., 2015). Transgenic lines harbouring this T-DNA were numbered with the code pKG10824.

Plant Transformation Method Agrobacterium transformation was performed by genotype-independent transformation of lettuce using Agrobacterium tumefaciens. Such methods are well-known in the art and e.g. taught in Curtis et al. Any other method suitable for genetic transformation of lettuce may be used to produce plants harbouring the desired T-DNA, such as described in Michelmore et al. (1987) or Chupeau et al. (1989).

### Results

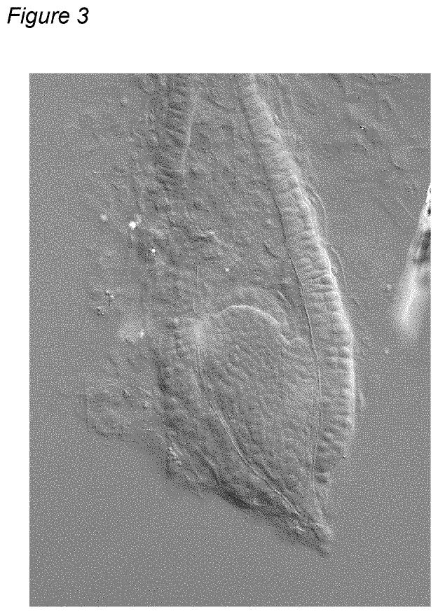

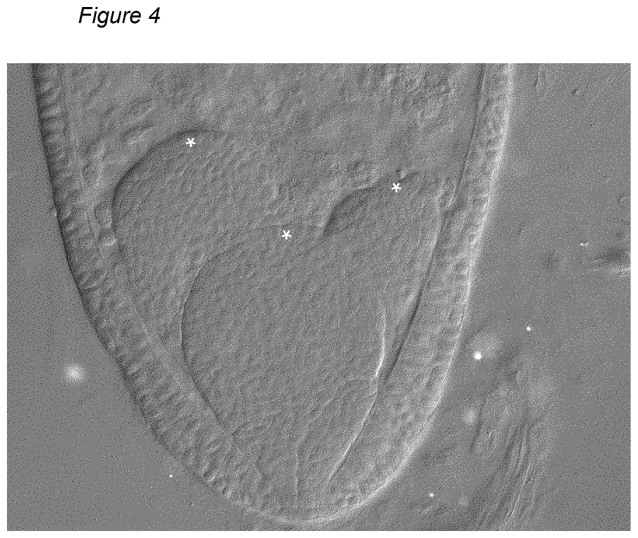

Plants that were positively tested for presence of the transgene as described under section “DNA construct” above, were evaluated for occurrence of parthenogenesis. As the trait is dominant, testing has been performed on the primary transformed plants (T0). In the absence of cross or self-fertilization, parthenogenetic egg cells develop into embryos. In order to prevent any fertilization of the plants harboring the transgene, plants were grown in a greenhouse and prior to microscopic observation, all flowers were manually emasculated. Emasculation was performed by clipping the involucre before the corolla has grown. Parthenogenesis can be detected in non-apomictic plants microscopically by Nomarski Differential Interference Microscopy (DIC) of cleared ovules. Here, the clearing method using chloral hydrate was applied; a method commonly used to clear ovules of plants for microscopic imaging. (e.g. Franks et al. 2016). At 75 hours post emasculation, flower buds were harvested and ovules were cleared with chloral hydrate. In all 7 evaluated transgenic lines multiple embryos were observed in these cleared ovules (see table 3, showing data for 5 of these lines). FIG. 3 shows an example of such observed embryos. In some single ovules, multiple embryos were observed (polyembryony). FIG. 4 shows an example of observed polyembryony. However, polyembryony was observed at a much lower frequency than single embryos. In non-emasculated transgenic lines, embryos could already be observed before completion of male gametogenesis and hence before fertilization. Also polyembryony was observed in some rare cases in these non-emasculated transgenic plants. In non-transformed control plants, which were emasculated and imaged in the same way, no embryos were observed at all.

TABLE 3
Effect of T-DNA construct encoding for the EC1.1 promoter driving
the Par allele gene Taraxacum officinale, in transgenic
lettuce lines. Shown numbers are from observations at 75 hours
post emasculation. In non-transformed controls, no embryos
were found at 75 hours post emasculation. In a single flower
bud about 25 ovules are present. The ovules that were visible
in a single microscopic plane were further analysed.

| | Number of | Number of |
|---|---|---|
| | emasculated | observed |
| Plant ID | flower buds | embryos |
| pKG10824-1 | 6 | 23 |
| pKG10824-8 | 6 | 20 |
| pKG10824-9 | 3 | 10 |
| pKG10821-16 | 5 | 32 |
| pKG10821-19 | 3 | 12 |

These results demonstrate that the Par allele gene of Taraxacum officinale is by itself sufficient to induce embryo formation in lettuce. This is a clear example of inducing parthenogenesis in lettuce with the Par allele gene of Taraxacum officinale as in the absence of cross or self-fertilization, egg cells developed into embryos. Similar results are expected when the lettuce homolog (SEQ ID NO: 22) is used for plant transformation in the same way, e.g. transforming said lettuce plant with a vector comprising a T-DNA region comprising a EC1.1 promoter of Arabidopsis thaliana (as in Sprunk et al. 2012) driving expression of a sequence encoding the lettuce homologue (SEQ ID NO: 22) with a 35S terminator and a neomycin phosphotransferase gene (nptII) for selection.

### Example 3

The gene of SEQ ID NO: 5 has homologs in parthenogenetic and non-parthenogenetic plant species. All such sequences were compared by means of multiple sequence alignments and variant calling, including 5′ and 3′ regulatory sequences. This was done in such a way to determine which differences are solely represented on parthenogenic plant species versions of the gene of SEQ ID NO: 5.

The inventors identified a Miniature inverted repeat transposable element (MITE) sequence or MITE-like of 1335 bp (defined herein by SEQ ID NO: 60) in the promoter sequence of the Par allele at a distance of 102 bp upstream (3′) of the start codon (SEQ ID NO: 2), which was identified to be absent in the sexual counterparts (SEQ ID NO: 7 and 12). This MITE or MITE-like sequence is expected to be indicative for, and may be causal for the parthenogenic phenotype for instance by being responsible for altering expression levels of the encoded protein.

These parthenogenic allele specific polymorphisms, insertions or deletions can be introduced by means of chemical mutagenesis or targeted gene editing of the sexual allele homologs of the parthenogenesis gene of this invention in non-parthenogenic plants. For instance, a promoter sequence of a PAR gene may be replaced by the promoter of the Taraxacum Par allele, i.e. SEQ ID NO: 2, or a MITE sequence may be introduced in the PAR gene of a non-parthenogenic plant at a position homologous to the MITE sequence in the Taraxacum Par allele as indicated above. Upon introduction of these parthenogenic allele specific polymorphisms, insertions or deletion, plants will obtain the parthenogenesis trait. Parthenogenesis can be detected in non-parthenogenic plants microscopically by Nomarski Differential Interference Microscopy (DIC) of ovules cleared with methyl salicylate (Van Baarlen et al. 2002). In the absence of cross or self-fertilization parthenogenetic egg cells develop into embryos. On plants harboring the above-mentioned specific polymorphisms, insertions or deletions at least a few of such embryos are found.

### Example 4

Triploid and tetraploid Taraxacum apomicts were crossed as pollen donors with diploid Taraxacum koksaghyz plants. The pollen donors themselves were obtained by crossing sexual Taraxacum kokaghyz with apomictic Taraxacum brevicorniculatum pollen donors. The apomixis genes thus originated from Taraxacum brevicorniculatum (Kirschner et al. 2012). Triploid progeny plants were tested for the presence of the Par allele and the Diplospory (Dip) allele (see WO2017/039452 A1), using a PCR-marker and for the production of apomictic seeds. Apomictic seed set was defined as the production of viable seeds on triploid plants without cross pollination.

Primers DIP_F (SEQ ID NO: 33 and DIP_R (SEQ ID NO: 34 were designed on diplospory gene VPS13 in order to amplify specifically the Dip allele. Using these primers, the presence of the Dip allele resulted in a PCR product of PCR 829 bp, whereas absence of this allele did not result in a PCR product. Primers PAR_F (SEQ ID NO: 35) and PAR_R (SEQ ID NO: 36) were designed on SEQ ID: 2 and SEQ ID: 4 in order to amplify any one of Par, par 1 and par 2 alleles. The presence of the Par allele could be distinguished by the length of the PCR product as shown in table 4.

TABLE 4
Amplicon length of PCR products of the parthenogenesis (Par) allele
and its sexual counter parts (par allele 1 and 2) using the primer pair
PAR_F (SEQ ID NO: 35) and PAR_R (SEQ ID NO: 36).

| | Par allele | par allele 1 | par allele 2 |
|---|---|---|---|
| Amplicon length (bp) | 2400 | 1071 | 1111 |

Fifty-six progeny plants were tested and a 100% correlation was observed between the presence of the Par allele and parthenogenesis as shown in Table 5 reported here below. No plants were observed that produced apomictic seeds and that were negative for the DIP and the PAR markers.

TABLE 5
Genotyping and phenotyping of progeny of a cross of triploid
and tetrapioid Taraxacum apomicts as pollen donors with
diploid Taraxacum koksaghyz plants.

| | | | | Seed set | |
|---|---|---|---|---|---|
| Number of | Progeny | Par | Dip | without | Seed |
| plants | ploidy level | allele | allele | pollination | germination |
| 56 | 3x | + | + | Yes | Yes |

It can therefore be concluded that the marker that was developed from the Par locus in Taraxacum officinale, also identifies the presence of parthenogenesis in a different species Taraxacum brevicomiculatum which is further proof that the Par allele causes parthenogenesis.

### Example 5

### Constructing a Gamma-Irradiation Deletion Population of Apomictic A68

Approximately 3×2000 seeds from clone A68 were gamma-irradiated with three different doses: one third with 250 Gy, one third with 300 Gy and one third with 400 Gy. In total 3075 plants from irradiated seeds were grown in pots in the greenhouse. After a vernalization period of two month below 10° C., the plants were again grown in the heated greenhouse. Over 90 percent of the plants flowered and produced seeds. Plants were classified whether or not they showed a Loss-of-Apomixis phenotype (LoA). Apomictic A68 plants produce seeds spontaneously and form large white seed heads, with a dark brown centre, where the seeds (achenes: one-seeded fruits) are attached to the receptacle. In the case of Loss-of-Apomixis phenotypes the centre of the seed head is lighter and often the seed heads are reduced in diameter. Finally 102 plants were identified as having Loss-of-Apomixis phenotypes.

Single dose dominant markers can be mapped in autopolyploid plants, using the method of Wu et al. (1992). In order to find AFLP markers (Vos et al. 1995) that were linked to the Par locus, a Bulked Segregant Analysis approach was used (Michelmore et al. 1991). Two contrasting DNA pools were constructed, pool A with DNA from 10 triploid PAR plants and pool B with DNA from 10 triploid non-Par plants, all progeny from the cross TJX3-20 (diploid sexual)×A68. Non-Par plants were carefully phenotyped for the absence of parthenogenesis using Nomarski DIC microscopy (Van Baarlen et al. 2002). For the Par-pool apomictic plants were used. 147 AFLP primer combinations (Vos et al. 1995) were screened for the presence of fragments in pool A and absence of fragments in pool B. Contrasting fragments in the pools were verified on individuals from the pools. Seventeen AFLP markers were used to construct a genetic map of the Par-locus chromosomal region based on the TJX3-20×A68 cross (76 plants). Fourteen of the 17 AFLP markers strictly co-segregated with the Par phenotype. This is an indication for suppression of recombination near the Par locus.

When one of the three homologous chromosomes is partly deleted, the single dose AFLP markers located on the deletion region will be lost. AFLP analyses of LoA plants indicated that a number of LoA plants had lost one or more AFLP markers that were genetically linked to the Par locus. LoA plants that lacked Par linked AFLP markers produced tetraploid offspring after crossing with a diploid pollen donor. This indicated that these LoA plants, although they had lost the apomixis phenotype, still were diplosporous, producing unreduced egg cells. These LoA plants could be ranked based on the number of Par genetically-linked AFLPs markers that they lacked. The number of lost AFLP markers is an indication of the size of the deletion. The AFLP marker that was most often lost in LoA plants was considered to be closest to the Par locus. Plant i34 lacked the fewest PAR linked AFLP markers and was thus considered to have the smallest deletion.

### Example 6

Genotype and Allele-Specific Expression of the Par Gene in the Megagametophyte in Apomict Taraxacum Plants Vs. Par Deletion and Sexual Plants

Cells and tissues from different developmental stages of the gametophyte were isolated by Laser-assisted Microdissection (LAM) using a SL μCut instrument which makes use of a solid state UV-A laser (wavelength approx. 350 nm) to cut the tissue (2001, Medical Micro Instruments, Glattbrugg, Switzerland), as described in Wuest et al. (2010) and Florez-Rueda et al (2020). Subsequently, transcriptome analyses were performed. RNA was extracted using PicoPure™ RNA isolation kit according to the instructions of the manufacturer (Thermo Fisher Scientific). To maintain the original expression differences between samples, the mRNA was, after reverse transcription to DNA, linearly amplified using CEL-seq and CEL-seq2 protocols, as described in Hashimshony et al. (2012) and Hashimshony et al. (2016).

Three plant lines were compared: 1. The triploid apomict A68 (short: APO) originating from The Netherlands, 2. tetraploid PAR deletion offspring from the crossing of the triploid deletion line i34 (a PAR deletion line, derived from A68, see example 5 above) with the diploid pollen donor FCH72 (Short: DEL) and 3. The diploid sexual plant FCH72 (short: SEX) originating from France.

Per plant line, five different developmental stages/tissue types were sampled (Table 6). For very young stages, single samples were analyzed. From the mature embryo sac, the central cells and the oocyte apparatus (egg cell and synergids) were sampled in triplicate. Together these represent nine samples per plant line (Table 6).

TABLE 6
Number of samples analysed per type and stage

| Sample Type | Stage | APO | DEL | SEX |
|---|---|---|---|---|
| Whole gametophyte | Young - Functional | 1x | 1x | 1x |
| | megaspore to 4-nuclei | | | |
| Whole gametophyte | Young - 8-nuclei to | 1x | 1x | 1x |
| Egg apparatus | cellularized embryo sac | 1x | 1x | 1x |
| Egg apparatus | Mature - 7 to 4 cell | 3x | 3x | 3x |
| Central cells | embryo sac | 3x | 3x | 3x |

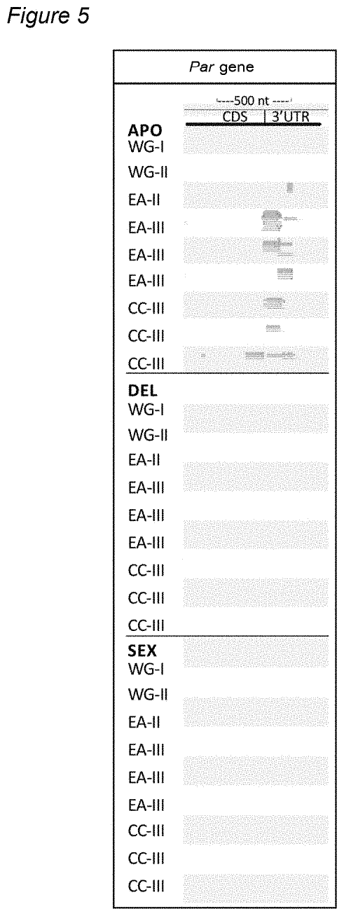

The linearly amplified DNA was sequenced on the Illumina Hiseq platform. Individual reads were mapped to the sequence of the Par gene (FIG. 5). The expression of the Par gene was not detected in any of the PAR deletion or SEX plants (all stages and tissues). In the APO line, reads specific to the Par gene were found in all samples of the mature gametophyte, both in the egg cell apparatus and in the central cell. Some transcription reads were also detected in one of the younger developmental stages of the apomict. In accordance with the 3′ end amplification bias of this method, most reads mapped to the 3′ end of the coding sequence and the 3′-UTR of the gene.

Thus, the Par gene is expressed in seven samples of the apomict, while it is not expressed in the seven samples of the deletion line, nor the seven samples of the sexual line, that are of comparable developmental state. This further underscores that the ectopic expression of the gene in the central cell and the egg cell apparatus is responsible for the loss of egg cell arrest and, consequently, the parthenogenetic development of the embryo.

As also indicated in example 3, the expression of the Par gene in the apomict in these cells may not be suppressed as in the sexual, possibly due to the influence of the MITE sequence in the promoter region. Because the MITE is large, it could physically interfere with the binding of transcription factors of the Par gene.

### REFERENCES

An et al. (1996) Plant J. 10, 107
Aoyama and Chua (1997) Plant Journal 11: 605-612
Asker, S. (1979) Progress in apomixis research. Hereditas 91(2): 231-240.
Asker, S. E. and Jerling, L. (1990) Apomixis in Plants. CRC Press, Boca Raton.
Ausubel et al. (1994) Current Protocols in Molecular Biology, Volumes 1 and 2, Current Protocols, USA
Bae T. W., Park R. H., Kwak Y. S., Lee H. Y. and Ryu S. B. (2005) Agrobacterium tumefaciens-mediated transformation of a medicinal plant Taraxacum platycarpum. Plant Cell, Tissue and Organ Culture 80: 50-57.
Baulcombe D. C. (1996) Plant Mol Biol. October; 32(1-2):79-88.
Barrell and Grossniklaus (2005) Confocal microscopy of whole ovules for analysis of reproductive development: the elongatel mutant affects meiosis II. Plant Journal 34: 309-320.
Bennetzen J. L. and Hall B. D (1982) J. Biol. Chem. 257: 3026-3031.
Bicknell and Koltunow 2004 Understanding apomixis: recent advances and remaining conundrums. The Plant Cell 16: S228-S245.
Bih et al. (1999) J. Biol. Chem. 274, 22884-22894.
Borevitz, J. O., Liang, D., Plouffe, D., Chang, H.-S., Zhu, T., Weigel, D., Berry, C. C., Winzeler, E. and Chory, J. (2003) Large-scale identification of single-feature polymorphisms in Arabidopsis. Genome Res. 13: 513-523.
Bortesi, L. and Fischer, R. (2015) The CRISPR/Cas9 system for plant genome editing and beyond. Biotechnology Advanced 33(1): 41-52.
Bruce M, Hess A, Bai J, Mauleon R, Diaz M G, Sugiyama N, Bordeos A, Wang G, Leung H, Leach, J. (2009) Detection of genomic deletions in rice using oligonucleotide microarrays. BMC Genomics: 10:129-140.
Catanach A S, Erasmuson S K, Podivinsky E, Jordan B R, Bicknell R. (2006). Deletion mapping of genetic regions associated with apomixis in Hieracium. Proc. Nat. Acad. Sci. 103: 18650-5.
Christensen et al. (1992) Plant Mol. Biol. 18: 675-689.
Chupeau et al. (1989) Transgenic plants of lettuce (Lactuca sativa) obtained through electroporation of protoplasts. Bio/Technology 7, 503-508.
Cordera et al. (1994) The Plant Journal 6, 141.
Cornejo et al. (1993) Plant Mol. Biol. 23, 567-581.
Cornelissen et al. (1986) EMBO J. 5, 37-40.
Crismani W. et al. (2013) J. Exp. Bot. 64:55-65.
Curtis I S et al. (1994) J. Exp. Bot. 45.10: 1441-1449.
Daniell, H. (2002) Molecular strategies for gene containment in transgenic crops. Nature biotechnology 20: 581-586.
de Pater et al. (1992) Plant J. 2, 834-844
Depicker A. and Van Montagu M. (1997) Post-transcriptional gene silencing in plants. Current Opinion in Cell Biology 9: 373-382.
Depicker et al. (1982) J. Mol. Appl. Genetics 1, 561-573.
Englbrecht et al. (2004) BMC Genomics, 5 (1): 39
Vielle-Calzada, J-Ph., B. L. Burson, E. O Bashaw, and M. A. Hussey 1995. Early fertilization events in the sexual an aposporous egg apparatus of Pennisetum ciliare (L.) Link, The Plant Journal 8(2):309-316. Dieffenbach and Dveksler (1995) PCR Primer: A Laboratory Manual, Cold Spring Harbor Laboratory Press.
Florez-Rueda et al (2020), Laser-Assisted Microdissection of Plant Embryos for Transcriptional Profiling, Methods Mol Biol, 2122:127-139
Foucu, F. (2006) Taraxacum officinale as an expression system for recombinant proteins: Molecular cloning and functional analysis of the genes encoding the major latex proteins. Thesis Rheinisch-Westfálischen Technischen Hochschule Aachen.
Franck et al. (1980) Cell 21, 285-294.
Franks R G (2016) Hum Press, New York, N. Y., 1-7.
Gardner et al. (1981) Nucleic Acids Research 9, 2871-2887.
Gatz, 1997, Annu Rev Plant Physiol Plant Mol Biol. 48: 89-108
Gielen et al. (1984) EMBO J 3, 835-845.
Guo et al, Scientific reports. 2017 Jun. 1; 7(1):2634.
Gould et al. (1991) Plant Physiol. 95, 426-434.
Grimanelli D. (2012) Curr. Opin. Plant Biol. 15:57-62.
Hashimshony, T., Senderovich, N., Avital, G. et al. CEL-Seq2: sensitive highly-multiplexed single-cell RNA-Seq. Genome Biol 17, 77 (2016).
Hashimshony T, Wagner F, Sher N, Yanai I. CEL-Seq: single-cell RNA-Seq by multiplexed linear amplification. Cell Rep. 2012; 2(3):666-673.
Helliwell and Waterhouse (2003) Methods 30(4):289-95.
Henikoff and Henikoff (1992) PNAS 89, 915-919.
Hermsen, J. G. Th. (1980) Breeding for apomixis in potato: Pursuing a utopian scheme. Euphytica 29:595-607.
Hesse et al. (1989) EMBO J. 8, 2453-2461.
Holmes, M (2018) Historical Studies in the Natural Sciences, 48 (1). pp. 1-23. ISSN 1939-1811
Hull and Howell (1987) Virology 86, 482-493.
Ikemura (1993) In “Plant Molecular Biology Labfax”, Croy, ed., Bios Scientific Publishers Ltd.
Itakura et al. (1977) Science 198, 1056-1063.
Kagale et al., (2010) Plant Physiology, 152: 1009-1134.
Keil et al. (1986) Nucl. Acids Res. 14, 5641-5650. Kirschner J, Štěpánek J, Černý, De Heer, P, and PJ van Dijk 2012. Available ex-situ germplasm of the potential rubber crop Taraxacum koksaghyz belongs to a poor rubber producer, T. brevicorniculatum (Compositae—Crepidinae). Genet. Resour. Crop Evol. DOI: 10.1007/s10722-012-9848-0
Klösgen and Weil (1991) Mol. Gen. Genet. 225, 297-304.
Klösgen et al. (1989) Mol. Gen. Genet. 217, 155-161.
Last et al. (1990) Theor. Appl. Genet. 81, 581-588.
Liu et al. (1995) Genomics 25(3):674-81.
Liu et al. (2005) Methods Mol. Biol. 286:341-8.
Love et al. (2000) Plant J. 21: 579-88.
Lutz K A et al. (2004) Plant J. 37(6):906-13.
Maillon et al. (1989) FEMS Microbiol. Letters 60, 205-210.
Ma, Xingliang, et al. “A robust CRISPR/Cas9 system for convenient, high-efficiency multiplex genome editing in monocot and dicot plants.” Molecular plant 8.8 (2015): 1274-1284.
Mc Bride et al. (1995) Bio/Technology 13, 362.
McPherson at al. (2000) PCR-Basics: From Background to Bench, First Edition, Springer Verlag, Germany.
Michelmore, R. W., Marsh, E., Seely, S. and Landry, B. (1987) Transformation of lettuce (Lactuca sativa) mediated by Agrobacterium tumefaciens. Plant Cell Rep. 6: 439-442.
Michelmore, R. W., Paran, I. and Kesseli, R. V. (1991) Identification of markers linked to disease resistance genes by bulked segregant analysis: a rapid method to detect markers in specific genomic regions using segregating populations. Proc. Natl. Acad. Sci. 88:9828-9832.
Morgan, R., Ozias-Akins, P., and Hanna, W. W. (1998) Seed set in an apomictic BC3 pearl millet. Int. J. Plant Sci. 159, 89-97.
Morris et al. (1999) Biochem. Biophys. Res. Commun. 255, 328-333.
Müller, K. J., He, X., Fischer, R., Prüfer, D. (2006) Constitutive knox1 gene expression in dandelion (Taraxacum officinale, Web.) changes leaf morphology from simple to compound. Planta 224: 1023-1027.
Nakamura et al. (2000) Nucl. Acids Res. 28, 292.
Nekrasov, Vladimir, et al. “Targeted mutagenesis in the model plant Nicotiana benthamiana using Cas9 RNA-guided endonuclease.” Nature biotechnology 31.8 (2013): 691.
Neuhaus & Rogers (1998) Plant Mol. Biol. 38, 127-144.
Odell et al. (1985) Nature 313, 810-812.
Oelmuller et al. (1993) Mol. Gen. Genet. 237, 261-272.
Oscarsson, L. “Production of rubber from dandelion—a proof of concept for a new method of cultivation.” 2015
Ozias-Akins, P. and P. J. van Dijk. (2007) Mendelian genetics of apomixis in plants. Annu. Rev. Genet. 41:509-537.
Park et al. (1997) J. Biol. Chem. 272, 6876-6881.
Plant Molecular Biology Labfax (1993) by R. D. D. Croy, jointly published by BIOS Scientific Publications Ltd (UK) and Blackwell Scientific Publications, UK.
Ríos G, Naranjo M A, Iglesias D J, Ruiz-Rivero O, Geraud, M, Usach, A and Talón M. (2008) Characterization of hemizygous deletions in Citrus using array-Comparative Genomic Hybridization and microsynteny comparisons with the poplar genome. BMC Genomics 9: 381-395.
Ross, M., LaBrie, T., McPherson, S., and Stanton, V. P. (1999). Screening large-insert libraries by hybridization. In Current Protocols. in Human Genetics, A. Boyl, ed (New York: Wiley), pp 5.6.1-5.6.32.
Sambrook and Russell (2001) Molecular Cloning: A Laboratory Manual, Third Edition, Cold Spring Harbor Laboratory Press, NY.
Sambrook et al. (1989) Molecular Cloning: A Laboratory Manual, Second Edition, Cold Spring Harbor Laboratory Press.
Savidan Y. (2001) Transfer of apomixis through wide crosses. In: Savidan Y, Carman J, Dresselhaus T, editors. The flowering of apomixis: From mechanisms to genetic engineering. Mexico: CIMMYT, IRD; pp. 153-167.
Shcherban et al. (1995) Proc. Natl. Acad. Sci USA 92, 9245-9249.
Sidorov V A et al. (1999) Plant J.19: 209-216.
Smith T F, Waterman M S (1981) J. Mol. Biol 147(1); 195-7.
Sprunck et al. (2012) Science 338.6110 1093-1097
Stam, M., Mol, J. N. and Kooter, J. M. (1997) The silencing of genes in transgenic plants. Annals of Botany 79: 3-12.
Sutliff et al. (1991) Plant Molec. Biol. 16, 579-591.
Tas, I. C. Q. and Van Dijk, P. J. (1999) Crosses between sexual and apomictic dandelions (Taraxacum) I. The inheritance of apomixis. Heredity 83: 707-714.
Tavladoraki et al. (1998) FEBS Lett. 426, 62-66.
Terashima et al. (1999) Appl. Microbiol. Biotechnol. 52, 516-523.
Vaeck et al. (1987) Nature 328, 33-37.
Van Baarlen, De Jong, J. H., and Van Dijk, P. J. (2002) Comparative cyto-embryological investigations of sexual and apomictic dandelions (Taraxacum) and their apomictic hybrids. Sex Plant Reprod 15: 31-38.
Van Den Broeck et al. (1985) Nature 313, 358.
Van Dijk, P. J. and Bakx-Schotman, J. M. T. (2004) Formation of unreduced megaspores (diplospory) in apomictic dandelions (Taraxacum) is controlled by a sex-specific dominant gene. Genetics 166, 483-492.
Van Dijk, P. J. and Schauer, S. E. https://www.keygene.com/wp-content/uploads/2018/07/apomixis-game-changer-in-breeding.pdf 2016Velten and Schell (1985) Nucleic Acids Research 13, 6981-6998.
Van Dijk, P. J., Rigola, D. and Schauer, S. E. “Plant breeding: surprisingly, less sex is better.” Current Biology 26.3 (2016): R122-R124.
Van Dijk, P. J., Tas, I. C. Q., Falque, M, and Bakx-Schotman J. M. T. (1999) Crosses between sexual and apomictic dandelions (Taraxacum). II. The breakdown of apomixis. Heredity 83: 715-721.
Van Dijk, P. J., Van Baarlen, P., and de Jong, J. H. (2003) The occurrence of phenotypically complementary apomixis-recombinants in crosses between sexual and apomictic dandelions (Taraxacum officinale). Sex. Plant Repr. 16: 71-76.
Velten et al. (1984) EMBO J 3, 2723-2730.
Verdaguer et al. (1998) Plant Mol. Biol. 37, 1055-1067.
Vielle-Calzada, J-Ph., B. L. Burson, E. C Bashaw, and M. A. Hussey 1995. Early fertilization events in the sexual an aposporous egg apparatus of Pennisetum ciliare (L.) Link, The Plant Journal 8(2):309-316.
Vielle-Calzada, J. P., Crane, C. F. and Stelly, D. M. (1996a) Apomixis: The asexual revolution. Science 274: 1322-1323.
Vijverberg, K. van der Hulst, R. Lindhout, P. and Van Dijk P. J. (2004) A genetic linkage map of the diplosporous chromosomal region in Taraxacum (common dandelion; Asteraceae). Theor. Appl. Genet. 108: 725-732.
Vos, P., Hogers, R., Bleeker, M., Reijans, M., Lee, Th. van der, Homes, M., Frijters, A., Pot, J., Peleman, J., Kuiper, M. and Zabeau, M. (1995). AFLP: a new technique for DNA fingerprinting. Nucl. Acids Res. 23: 4407-4414.
Wesley et al. (2003) Methods Mol Biol. 236:273-86.
Wesley et al. (2004) Methods Mol Biol. 265:117-30.
Wong et al. (1992) Plant Molec. Biol. 20, 81-93.
Wu K K1, Burnquist W, Sorrells M E, Tew T L, Moore P H, Tanksley S D (1992) The detection and estimation of linkage in polyploids using single-dose restriction fragments. Theor. Appl. Genet. 83: 294-300.
Wuest S E, Vijverberg K, Schmidt A, et al. Arabidopsis female gametophyte gene expression map reveals similarities between plant and animal gametes. Curr Biol. 2010; 20(6):506-512.
Zhang et al. (1991) The Plant Cell 3, 1155-1165.

1. A nucleic acid that is associated with parthenogenesis in plants, wherein the nucleic acid comprises at least one of:
(a) a gene that encodes a protein having an amino acid sequence of SEQ ID NO: 1, 6 or 11;
(b) a promoter having the nucleotide sequence of SEQ ID NO: 2, 7 or 12;
(c) a coding sequence having the nucleotide sequence of SEQ ID NO: 3, 8 or 13;
(d) a 3′UTR having the nucleotide sequence of SEQ ID NO: 4, 9 or 14;
(e) a gene having the nucleotide sequence of SEQ ID NO: 5, 10 or 15;
(f) a variant or fragment of any one of (a)-(e).

2. The nucleic acid of claim 1, wherein the nucleic acid comprises a chimeric gene, genetic construct or nucleic acid vector.

3. A protein that is associated with parthenogenesis in plants, wherein the protein:
(a) is encoded by the nucleic acid of claim 1;
(b) has an amino acid sequence of SEQ ID NO: 1, 6 or 11; and/or
(c) is a variant or fragment of (a) and/or (b).

4. A plant or plant cell not being of the species Taraxacum officinale sensu lato, comprising the nucleic acid of claim 1.

5. The plant or plant cell according to claim 4, wherein the plant or plant cell is from a family selected from the group consisting of Brassicaceae, Cucurbitaceae, Fabaceae, Gramineae, Solanaceae, Asteraceae (Compositae), Rosaceae and Poaceae.

6. The plant or plant cell according to claim 4, wherein the plant or plant cell comprises the nucleic acid by genetic modification or by introgression.

7. The plant or plant cell according to claim 4, wherein the plant or plant cell is capable of parthenogenesis.

8. The plant or plant cell according to claim 4, wherein the plant or plant cell is further capable of apomeiosis.

9. A plant or plant cell not being of the species Taraxacum officinale sensu lato, comprising the protein of claim 3.

10. The plant or plant cell according to claim 4, wherein the plant or plant cell is from a family selected from the group consisting of Brassicaceae, Cucurbitaceae, Fabaceae, Gramineae, Solanaceae, Asteraceae (Compositae), Rosaceae and Poaceae.

11. The plant or plant cell according to claim 10, wherein the plant or plant cell is further capable of apomeiosis.

12. A seed, plant part or plant product of a plant or plant cell of claim 4.

13. A seed, plant part or plant product of a plant or plant cell of claim 9.

14. A method for producing a parthenogenetic plant, comprising:
(a) introducing in one or more plant cells a nucleic acid of claim 1 that is capable of inducing parthenogenesis;
(b) selecting a plant cell comprising the nucleic acid; and
(c) regenerating a plant from said plant cell.

15. The method according to claim 14, wherein the nucleic acid is integrated in the genome of the plant cell.

16. A method for producing an apomictic F1 hybrid seed, comprising:
(a) cross-fertilizing a sexually reproducing first plant with the pollen of a second plant to produce F1 hybrid seeds, wherein the second plant comprises a nucleic acid of claim 1, and wherein the first and/or second plant is capable of apomeiosis.

17. The method according to claim 16, further comprising:
(b) selecting from the F1 seeds that comprise the apomictic phenotype.

18. The method according to claim 17, wherein the selecting is by genotyping.

19. The method according to claim 16, further comprising:
(c) growing at least one F1 plant from the F1 hybrid seed.

20. A plant, seed, plant part or plant product obtainable by the method of claim 14.


SEQUENCE LISTING (60 sequences)

SEQ ID NO: 1 - protein, 170 aa, Taraxacum officinale
MADNGNTGRQKDDDGGHDGPRQNPTTPPSPSRTPRRPRRNTSPPKHSPGASSSTMPAPPTPPAPTGITGASSSSVGTNIISFIPPKTKRTKSVICPICNKDMCHEKALCGHIRWHTQEERLAASIAIARALSSNVVVSGNGDEDEGPSKKYKLPDLNKSPPPEEEDEDAA

SEQ ID NO: 2 - DNA, 1900 bp, Taraxacum officinale
taaggccataacaaggttatttgaacattcgcctaaaccctaaaccaactatatctattcggatttgttacgagttttagagtttctagaaaatcagttatatatcgattaggcttggttctaggttataagaaaaaatgattctctatccggtatgacatagagacaaaccggtctacttttataagaaaatcatatttgaaacaatgtctagttaataattaattaatatgtgcatgtttaaatttagaatatcctgaatttatatttagtgcgcaaccctatgtaagatcgaactaattttaaaccgtaaccagacatgcatgtttgatcagtaataaaaaggctattatcgtcgaaaatattaaaacaaaatataatatcttatttggcgccatttcatgtcaacggttgaaagcaagttaccaacgcctccaaccgatcatttctccggtaacaaccaactgctaccagggccggccagaaggctgtgcggcttgggctgtggcacaggggcagcagatttagtccatttattatggtctgctatttatttgggtccaataagatcagggcactaaaactcaaatacatcaattcaagtcggcggcaaaagaataaaaagcgccaatttttagaagacaaggagacagcacaataatcatgcttttccaatggccaattaattttagcctttgtgaggtataaatatgaatcactttttctccaaatatcattattgtaaactagtaaagagcgtaaattgttttatcgctaatcttcttctttttcaaatataattgtttgattctttgtagtaggtacgttaagttttatatcattcgtcctggaaaccttttcttatgccccttcaatttatgaatttgtgttttgtattgtcttgctattaatccaatgcaaactaattctgccgcatcgatctcacagagttggagcagcttaaaaggagttttggggcaacttaaaatgagggtagcttttttgttgttgttggtctactatttactttggtccttatcgttttagttttgtgttcattgaggatttgaatgactttgaacacaagtttataaaactaagtttttgtgattctaaatttttgatgtgtgcaaccgactaaatgaatataaaattctgatttgtgtatttctgatttctgatttaccggttattcattatgtttttctggatctcatatgaatttagtatataaaaatctcaggttcttcttactcagtctagtatattttggcattggggatcagggttaaaatcatcatttttctaagcactaacaagctttaaaaccatcaattaaaagttagttctccatacctggaattgtttattgctttagtattcatttcatagtgtcaataccttacaactgtttattgttttcaaatgtttactgttttgcactttagttgtttttagcatgtttcaactttcaaagttaattgacgatagtgtcatatacgaaatatcatcactcatacaaaattaaactggatagaattttttccgatgaaatctatttttaaatagttacgaatataataggagacttaatatgtttgtttcaaaattacacaaaaacaccatggaaatgggcattagtttgtataagcatccttttaaatataaagctttgttacattgatgccttctaaatatataatattgtaatgatttgtagtaaacaccatggaaatgggcattattaagatgtttcgcccagggcatgaaaaactatcggaccggccctgactgctactaataaccgccaccgcaaaaacctataaatacaacttccatcacaattctgtcccctactcccatttcaatccaaaaaaaggctacacagacatatacagcg

SEQ ID NO: 3 - DNA, 513 bp, Taraxacum officinale
atggcagataatggcaacaccggccgtcaaaaggatgacgacggtggccatgatggaccacgccaaaacccaactactccaccctccccttcccgcacccctcgaagaccaaggcggaacacatcaccgcccaaacattctccgggggcgtcttcaagcaccatgccagcgccgcctactccccctgcgccgacgggaatcaccggtgcttctagttcttctgtgggtactaatataatttcatttattccacccaaaaccaaaagaacgaagtcggtgatctgcccgatctgcaacaaagatatgtgccatgagaaggcgctgtgtggccacatccggtggcatacacaggaggaaagattggcggccagcatcgctatagcaagagcgctatcttctaacgttgttgtttctggcaatggcgatgaagatgaaggtccatctaaaaagtataaactcccggacctgaacaaatctccaccgccggaggaggaggacgaggacgctgcctga

SEQ ID NO: 4 - DNA, 5356 bp, Taraxacum officinale
ttttcttgcgggagaggcatgcatgtatgtgtggcctttttgcaataatactatgcatgggagatgcataaagtttatctttattttttttagtttaataatgtgtggcaaactttatatcatttatgatttcggccatgtactgctacatcgttctaggatggttattccctagaatttgctttatgtatttgttttctttgttaattcttctcttccttaaacgttatttcatgtgaattgcgatgtcatgatattgcaggaatgtaacttagaactcaacgataaaaagtgtgttgagatcgtggaagttgatcatttatcttgaattcataagataagcaacatgagataggataattaattactgtggggatttctttttatattcatattattcgttgtaatgcaaatttttaagtagaataacgaatgtgtcttattatttacggttaaatgcaaaagatcattaacatatctccattgatttgtttactgatgtattctcatgtgttctgttgctaaattctattttgaaaagtatattcaattaaagcaatgcattttagatataaagcaactccataacttataattgtgtagatttttaccaattcaatgtcttaaaagaaaaataaaaattgaaagtttaattaattgccatagtgatcccgttcccgtcaattatgtgatcaatttttaccagagcaaaaccccacatttcttgtagtgaacaatctgtggatgataaccgatcgaagacttttgtggggatttcactaaccaactatttggctctcattaattactattagttatataaggccatcccaagtattttggcaatattcaaaacgagtaaactataaaattacacttttggttcttgtatttctaacttttagaattatgtatttggttcttatagttttttatttgtatgaattacattacttttggtccacaatattttttattttctataattccatttttagtcattgtagttttaatttgcaataaaaaattaatttatatttttaatccatatatatagttggcaatattctatattttaggttcagattgtatcttttataaaaaagagtcttaaaagactaaaccttttggatccttatagttttcacaatcacttgtgatttttttaatgaacatatcatatttcttaaattacatcagtaatctacgtataacatttaatataattttatttaatcagaaatataggattcgtataataactttgctgaacttaggcttcatttttaacaaaggataatgcatcatgcatgacataaaaatttgtaatatttctaatgtgcatgcactacaagaacctgacccttcatatacattaatgaatgattctcttttcttatttaatcttagcaagcggtttggataattgtctgcacaatattaagaattgcactagctagctaggggagtccatgacgtaataagcaaattaactagaggacttgtaggaggcttgatcacttcattgtatatctaatggcactatcgatatatgattggcttgtgtgtatctatggaatttaacactattaaaaaatatatctttaatgacaatcaacaacacacagtaatttatgacttacaaaatcatgtgtgattaaaaaaaatgtcatgttttttaaaattatagaggatttacgacttacataattatgtgtcataattatatgtcatcttgtgtaatatttatgacttacaaaaatgtatgtcatgttttatcatcataaaatctataaaaaaaaatagaaacacgaggatactttcagtgtatgttatctgtaaaaaaaaataaaaactcggaggacactaaaagtgtagaaacttgacagcataaacgaatttatttaccttcatcaaaacttattttacttcaatctctgaaccagctcccttcattaatctcttcgagatgtaggttgattttaatgctaaatgaacttttcaaatgtataaataaaacttttctgtgccagatttgacattgatggagatgaagatacacaagtgaattcacctttcaatgatcatactgacataccaagtactcctatcccctctccacctccacaaaacgaacctcaatttcaaaaacgtaactatcatttaaatatttaatcatttttcaattggattcatgcggtaatagaagttactggactttaaactttttgtagctgatgagattcatgagcaacagtccatggacattttccaaatcaacaagtttctgatgaggacaacgtaaaaaaagtttttcaattattaaacatatgattttggtttttaaaagataattaattatggaaaatcttcagaaaagtctcaatactttgggtttattaatgaaaaagtctcaaaaaaaaatttgaataaaaaagtccataaaattggataaattattcgatttagcccaatttgccaagtaaatctggtaaaattaggctaaatcgaataatttagccagtttggtgggcctttttgcgcaaaaacattattttgggacttaatcgttaattttccccaaatttcagtttatttacagtcttggagtaaaaccgaacctagtgcgctaattttggcttttggtgtttggaattatttgggtttattgggctcggtttctgaacgagtttgatttttggtttcttgattcaattttttggttttggggttgaacccatggattttcgatttggtttcggctttgataatatttttgtgcaatcgttcaaaatatccgagttagattgggtttaaatgttatcaaaaccgaaaccgagggtagaaccccaaaactgaacccaatcgaaaatccataggttgaaccccagaaccgaacccaataaactctaaaaaccaaaagccaaaattaaccgataaaaaacttaaaatacatgtttgttaaatctagtaaggtgtttttgacactttatgtaaattatagaaaaaaatggattaactgtttgcttattaagataaaaacgacgtaaaaatagaattggattagaaatgaaccatttttttacatcgtttactttatttactaggttataacccgtgtattacacgggttgacgataaaaaattgtataaaaaaacaacatataatggtaagtttgtaatcgtacatgggttgcttcaattttaatttaaaaattgtaattccaaatgaaatatatgtaggatgatataacatacttgtaaatgtaaatgagagtttgttggcatttcatacatttagcccatcaaatcaaacgcaaaaaatctttttaatcctataatacatgtggcacaaatatacgattagcatttcgctgagagagaggctttaagtgaggatgtgaaaaagaaggaacatagcaacttggaatttaaagatcaaaaaaccttgaaaggcttttggaatgtaaaaatatagaaatgaataaatgtggcttcttgtatgtatcaagagtaggacctgtattagaaacataaaaaatttgtgtctaataaaaatcaacaaaaacagaacgtaaacgaaagaagttattggtaaaaaaagatttcaaaaatagtttttttttgaaccggttcaaaaatagttattttacttatttatccatttaatccaaacttttttttgtcaaatatctcatggaattagttggctgcgtcactcttgtcactgggcgttataacttttgccttgtggtttgcaacaccaccttaaataacatagacaaataacattaaatatcatgataatacataataactcacaaaagatgcattctatagaacggtataattgattagaacaaaataatctaaatagtttatgaggattaccttgataattcccttggagtgaatactatccacgatgtcatcttccaaaggtattcaatggcttttattagaacccaatttcagaattacattgatgccacagttacattcactaaatatcttcctcttaggcttttagtttgactatattttaaccattttcacatcacactttaaccaccccctttggttattaatgtcaaaaggaacaaaactcacaaatcaaatgcgtggtgtttatactattggtacaatgtttataaatttcttaaaaatgagcacaatttcaaaaaacttaaacaaatgtcaatggtcaccatgtggtattaatattataaagttctaaaaataccattccaccccaacatggtactaaccagttagaaaatcaagaaacagaaacatactctttaatctaaaacaatataatatagggatgtgttatgagtttagtttcatctaaccatgaattcatataggattgtacgtttctcctcgaaattgcaaagaattttgttcgactaagcaatctacatattaatggatttgtatggtttctcggtttgtaggtaagttaagagctaccaggaaagagaaaatttgcaccataggtactaaacacattctacaatggtggctttaacgtgagagagaatagagaggtcgaaacatatttccgttaaataagcaaacataagtttctatttgggatcaataagtttcttcctatagtggtaaatggatctgtacttgttaagctcttaaacatatttgttgtcactgttcacttcctattttgcagtgctcttctattacttacattcttcttatgacattcttttaataaggaaaaataaccacttattctagtattctattgttttgttagagtcaatatcagataagaacctaaaaaggtgggacaaattgataacttaatcaatatatcaaactgtcatatgtctcaagtctcaacccaaattgatatgtttaagaaaaattcagaaaagatatgtccatgggacatgtgcatgtatatgtataagtgtacattcctaaaaaaagtaggctcatatgacatgagcatgtatgtgcttattgttataaagattacaaattttcttaaaaatgaacacaatttcaaaatacctataccaaagcccatgatcaacatagcatctgatattaatattataaagctctaaacaaatctaccccgacatcaagtggtacttacaagttggaaaatcaagatacaaaaacatagcttttaatctaagtaaaaacacaataatatagggatatgacctatatgcatgtttaaaatcttaagtctgtaaagattgataggattggaaaactcttctattcaacacaagaaaagaattacagtaggaggagggactctcacactcaactactacaaaaaaaccaaccctcacttactaacactctcacactagtg

SEQ ID NO: 5 - DNA, 7769 bp, Taraxacum officinale
taaggccataacaaggttatttgaacattcgcctaaaccctaaaccaactatatctattcggatttgttacgagttttagagtttctagaaaatcagttatatatcgattaggcttggttctaggttataagaaaaaatgattctctatccggtatgacatagagacaaaccggtctacttttataagaaaatcatatttgaaacaatgtctagttaataattaattaatatgtgcatgtttaaatttagaatatcctgaatttatatttagtgcgcaaccctatgtaagatcgaactaattttaaaccgtaaccagacatgcatgtttgatcagtaataaaaaggctattatcgtcgaaaatattaaaacaaaatataatatcttatttggcgccatttcatgtcaacggttgaaagcaagttaccaacgcctccaaccgatcatttctccggtaacaaccaactgctaccagggccggccagaaggctgtgcggcttgggctgtggcacaggggcagcagatttagtccatttattatggtctgctatttatttgggtccaataagatcagggcactaaaactcaaatacatcaattcaagtcggcggcaaaagaataaaaagcgccaatttttagaagacaaggagacagcacaataatcatgcttttccaatggccaattaattttagcctttgtgaggtataaatatgaatcactttttctccaaatatcattattgtaaactagtaaagagcgtaaattgttttatcgctaatcttcttctttttcaaatataattgtttgattctttgtagtaggtacgttaagttttatatcattcgtcctggaaaccttttcttatgccccttcaatttatgaatttgtgttttgtattgtcttgctattaatccaatgcaaactaattctgccgcatcgatctcacagagttggagcagcttaaaaggagttttggggcaacttaaaatgagggtagcttttttgttgttgttggtctactatttactttggtccttatcgttttagttttgtgttcattgaggatttgaatgactttgaacacaagtttataaaactaagtttttgtgattctaaatttttgatgtgtgcaaccgactaaatgaatataaaattctgatttgtgtatttctgatttctgatttaccggttattcattatgtttttctggatctcatatgaatttagtatataaaaatctcaggttcttcttactcagtctagtatattttggcattggggatcagggttaaaatcatcatttttctaagcactaacaagctttaaaaccatcaattaaaagttagttctccatacctggaattgtttattgctttagtattcatttcatagtgtcaataccttacaactgtttattgttttcaaatgtttactgttttgcactttagttgtttttagcatgtttcaactttcaaagttaattgacgatagtgtcatatacgaaatatcatcactcatacaaaattaaactggatagaattttttccgatgaaatctatttttaaatagttacgaatataataggagacttaatatgtttgtttcaaaattacacaaaaacaccatggaaatgggcattagtttgtataagcatccttttaaatataaagctttgttacattgatgccttctaaatatataatattgtaatgatttgtagtaaacaccatggaaatgggcattattaagatgtttcgcccagggcatgaaaaactatcggaccggccctgactgctactaataaccgccaccgcaaaaacctataaatacaacttccatcacaattctgtcccctactcccatttcaatccaaaaaaaggctacacagacatatacagcgatggcagataatggcaacaccggccgtcaaaaggatgacgacggtggccatgatggaccacgccaaaacccaactactccaccctccccttcccgcacccctcgaagaccaaggcggaacacatcaccgcccaaacattctccgggggcgtcttcaagcaccatgccagcgccgcctactccccctgcgccgacgggaatcaccggtgcttctagttcttctgtgggtactaatataatttcatttattccacccaaaaccaaaagaacgaagtcggtgatctgcccgatctgcaacaaagatatgtgccatgagaaggcgctgtgtggccacatccggtggcatacacaggaggaaagattggcggccagcatcgctatagcaagagcgctatcttctaacgttgttgtttctggcaatggcgatgaagatgaaggtccatctaaaaagtataaactcccggacctgaacaaatctccaccgccggaggaggaggacgaggacgctgcctgattttcttgcgggagaggcatgcatgtatgtgtggcctttttgcaataatactatgcatgggagatgcataaagtttatctttattttttttagtttaataatgtgtggcaaactttatatcatttatgatttcggccatgtactgctacatcgttctaggatggttattccctagaatttgctttatgtatttgttttctttgttaattcttctcttccttaaacgttatttcatgtgaattgcgatgtcatgatattgcaggaatgtaacttagaactcaacgataaaaagtgtgttgagatcgtggaagttgatcatttatcttgaattcataagataagcaacatgagataggataattaattactgtggggatttctttttatattcatattattcgttgtaatgcaaatttttaagtagaataacgaatgtgtcttattatttacggttaaatgcaaaagatcattaacatatctccattgatttgtttactgatgtattctcatgtgttctgttgctaaattctattttgaaaagtatattcaattaaagcaatgcattttagatataaagcaactccataacttataattgtgtagatttttaccaattcaatgtcttaaaagaaaaataaaaattgaaagtttaattaattgccatagtgatcccgttcccgtcaattatgtgatcaatttttaccagagcaaaaccccacatttcttgtagtgaacaatctgtggatgataaccgatcgaagacttttgtggggatttcactaaccaactatttggctctcattaattactattagttatataaggccatcccaagtattttggcaatattcaaaacgagtaaactataaaattacacttttggttcttgtatttctaacttttagaattatgtatttggttcttatagttttttatttgtatgaattacattacttttggtccacaatattttttattttctataattccatttttagtcattgtagttttaatttgcaataaaaaattaatttatatttttaatccatatatatagttggcaatattctatattttaggttcagattgtatcttttataaaaaagagtcttaaaagactaaaccttttggatccttatagttttcacaatcacttgtgatttttttaatgaacatatcatatttcttaaattacatcagtaatctacgtataacatttaatataattttatttaatcagaaatataggattcgtataataactttgctgaacttaggcttcatttttaacaaaggataatgcatcatgcatgacataaaaatttgtaatatttctaatgtgcatgcactacaagaacctgacccttcatatacattaatgaatgattctcttttcttatttaatcttagcaagcggtttggataattgtctgcacaatattaagaattgcactagctagctaggggagtccatgacgtaataagcaaattaactagaggacttgtaggaggcttgatcacttcattgtatatctaatggcactatcgatatatgattggcttgtgtgtatctatggaatttaacactattaaaaaatatatctttaatgacaatcaacaacacacagtaatttatgacttacaaaatcatgtgtgattaaaaaaaatgtcatgttttttaaaattatagaggatttacgacttacataattatgtgtcataattatatgtcatcttgtgtaatatttatgacttacaaaaatgtatgtcatgttttatcatcataaaatctataaaaaaaaatagaaacacgaggatactttcagtgtatgttatctgtaaaaaaaaataaaaactcggaggacactaaaagtgtagaaacttgacagcataaacgaatttatttaccttcatcaaaacttattttacttcaatctctgaaccagctcccttcattaatctcttcgagatgtaggttgattttaatgctaaatgaacttttcaaatgtataaataaaacttttctgtgccagatttgacattgatggagatgaagatacacaagtgaattcacctttcaatgatcatactgacataccaagtactcctatcccctctccacctccacaaaacgaacctcaatttcaaaaacgtaactatcatttaaatatttaatcatttttcaattggattcatgcggtaatagaagttactggactttaaactttttgtagctgatgagattcatgagcaacagtccatggacattttccaaatcaacaagtttctgatgaggacaacgtaaaaaaagtttttcaattattaaacatatgattttggtttttaaaagataattaattatggaaaatcttcagaaaagtctcaatactttgggtttattaatgaaaaagtctcaaaaaaaaatttgaataaaaaagtccataaaattggataaattattcgatttagcccaatttgccaagtaaatctggtaaaattaggctaaatcgaataatttagccagtttggtgggcctttttgcgcaaaaacattattttgggacttaatcgttaattttccccaaatttcagtttatttacagtcttggagtaaaaccgaacctagtgcgctaattttggcttttggtgtttggaattatttgggtttattgggctcggtttctgaacgagtttgatttttggtttcttgattcaattttttggttttggggttgaacccatggattttcgatttggtttcggctttgataatatttttgtgcaatcgttcaaaatatccgagttagattgggtttaaatgttatcaaaaccgaaaccgagggtagaaccccaaaactgaacccaatcgaaaatccataggttgaaccccagaaccgaacccaataaactctaaaaaccaaaagccaaaattaaccgataaaaaacttaaaatacatgtttgttaaatctagtaaggtgtttttgacactttatgtaaattatagaaaaaaatggattaactgtttgcttattaagataaaaacgacgtaaaaatagaattggattagaaatgaaccatttttttacatcgtttactttatttactaggttataacccgtgtattacacgggttgacgataaaaaattgtataaaaaaacaacatataatggtaagtttgtaatcgtacatgggttgcttcaattttaatttaaaaattgtaattccaaatgaaatatatgtaggatgatataacatacttgtaaatgtaaatgagagtttgttggcatttcatacatttagcccatcaaatcaaacgcaaaaaatctttttaatcctataatacatgtggcacaaatatacgattagcatttcgctgagagagaggctttaagtgaggatgtgaaaaagaaggaacatagcaacttggaatttaaagatcaaaaaaccttgaaaggcttttggaatgtaaaaatatagaaatgaataaatgtggcttcttgtatgtatcaagagtaggacctgtattagaaacataaaaaatttgtgtctaataaaaatcaacaaaaacagaacgtaaacgaaagaagttattggtaaaaaaagatttcaaaaatagtttttttttgaaccggttcaaaaatagttattttacttatttatccatttaatccaaacttttttttgtcaaatatctcatggaattagttggctgcgtcactcttgtcactgggcgttataacttttgccttgtggtttgcaacaccaccttaaataacatagacaaataacattaaatatcatgataatacataataactcacaaaagatgcattctatagaacggtataattgattagaacaaaataatctaaatagtttatgaggattaccttgataattcccttggagtgaatactatccacgatgtcatcttccaaaggtattcaatggcttttattagaacccaatttcagaattacattgatgccacagttacattcactaaatatcttcctcttaggcttttagtttgactatattttaaccattttcacatcacactttaaccaccccctttggttattaatgtcaaaaggaacaaaactcacaaatcaaatgcgtggtgtttatactattggtacaatgtttataaatttcttaaaaatgagcacaatttcaaaaaacttaaacaaatgtcaatggtcaccatgtggtattaatattataaagttctaaaaataccattccaccccaacatggtactaaccagttagaaaatcaagaaacagaaacatactctttaatctaaaacaatataatatagggatgtgttatgagtttagtttcatctaaccatgaattcatataggattgtacgtttctcctcgaaattgcaaagaattttgttcgactaagcaatctacatattaatggatttgtatggtttctcggtttgtaggtaagttaagagctaccaggaaagagaaaatttgcaccataggtactaaacacattctacaatggtggctttaacgtgagagagaatagagaggtcgaaacatatttccgttaaataagcaaacataagtttctatttgggatcaataagtttcttcctatagtggtaaatggatctgtacttgttaagctcttaaacatatttgttgtcactgttcacttcctattttgcagtgctcttctattacttacattcttcttatgacattcttttaataaggaaaaataaccacttattctagtattctattgttttgttagagtcaatatcagataagaacctaaaaaggtgggacaaattgataacttaatcaatatatcaaactgtcatatgtctcaagtctcaacccaaattgatatgtttaagaaaaattcagaaaagatatgtccatgggacatgtgcatgtatatgtataagtgtacattcctaaaaaaagtaggctcatatgacatgagcatgtatgtgcttattgttataaagattacaaattttcttaaaaatgaacacaatttcaaaatacctataccaaagcccatgatcaacatagcatctgatattaatattataaagctctaaacaaatctaccccgacatcaagtggtacttacaagttggaaaatcaagatacaaaaacatagcttttaatctaagtaaaaacacaataatatagggatatgacctatatgcatgtttaaaatcttaagtctgtaaagattgataggattggaaaactcttctattcaacacaagaaaagaattacagtaggaggagggactctcacactcaactactacaaaaaaaccaaccctcacttactaacactctcacactagtg

SEQ ID NO: 6 - protein, 227 aa, Taraxacum officinale
MSTVKSKLSTPPTDHFSGNHQLLLITATAKTYIYNFHHNSVPYSHFNPKKATQTYTAMADNGNTGRQKDDDGGHDGGRPNPTTPPSPSRTPRRPRRTTSPPKHSPGASSSTMPAPPTPPAPTGITGASSSSVGTNIISFTPPKTKRTKSVICPICKKDMCHEKALCGHIRWHTQEERLAASIAIARALSSNVVVSGNGDEDEGPSKKYKLPDLNKSPPPEEEDEDAA

SEQ ID NO: 7 - DNA, 14519 bp, Taraxacum officinale
ctgacttggccaacattttttgttcttccgatcccttcgtaacaatcagtttaagcacaattgaattatatggagataaataggtgaaatttggaagacgaggatgggagagtgcgaagagccaaagaagagaaatgtgggatagcggccggccggttgttaatagaagtgcttagatcttggagaccttctaatgcttatggaggaaccagatgcttcaatttgtaaatctgcgctgcccacgtagaatattgaaaaagaaaatcaaatcgatcaataatgcgaccatatactttaggatttcccacttgtataattctcttattcattttcaaatctttatctaaattaaggatatcggcaaaaatataatcctaattgtttttcaaaatgataaggattataaggtaacttaattcttgttttattcatatttaattattattgttttttatcaatgttttaaaacccgggttttgagtcaacccggtcttgtgaaaaatcccgggtcagccggtcagctgatctaccggttcaatatatttaaataaaatataaattttcatatagaatttgagttttaggctaagaaaatcgggttttgggctaaggaaaatcgggtttaagttttgggctaatggattttcgggtcaataacgtttttttccgttttttagatcggttcgaccgcgttttgtgtgaaacccggccgggttgatccgagtcaataattggcaacaacccggtatatgttgacccgctcttgtccccgggtcccggttcaaccggttggatcggccgggttgaccctggttttaaaacactgttttttatacaatatatttgtttttgtttttagttttgtttttgtttttgattttgtagatatatttttaattttatttttattttgtataaatatagtcattacaaccgattatttgtaagttgagtcggtcaaaacgaactttctgataattcttatacatttttcggcgaaacatgaatattttgatcaaccaatatgcttttttctaggttttttaaataaaccgcacgttcaattgcatattcgcaaacaatagaactcgaaaaccctaatcgagcttgttttatagaaatctgggttgagtgcatacataataattaataactgtgaaaaagtttgttttaacatgctaaacctatcgatagtcggttataataattatatgtaaacaaaatcagaaacaataatgatatatgtcacccaaaaaaatagttactaaatatatacaaaatcaaaaacaacagtaatttcttttacaaaaaaaagagaacaatagtgactaactttagaaagaaaaaaaaagaactaaattactcttataaatcattatcttttatataaattggtaaattggtcatgtaatctagaatgtgaattgcggaatgctttagtcacattttgaacaaactggtaattattaatttcgataaaagttcttcaagaatagtttggtcacattttggacgacaatggtgtgttttccatagtaagcaatacgtaatgagttcgcttaataggaaaatagatctaaagcttaatgaaatcatatgtataagtaacatatatttttcacacgttacattgtttgtaacgtcctcaatttttttaatatatataactaggtacactctaaggtcgtgctaagcacgacccacgaatggtaatttaggtaatttttcatccttgactaaaatacattatgctaataacaaagaattaagaatacaatgctataatgcataagtaaataaaagtacatgatatttctcacatttagtctttatacatagaaaacttacactggccatttgcaaaaaaacattaaatctccctaaaaagtagagtttacaaaatgaaagcttgtttttatagcaaacaataatcaatatgtgaagctatagtatagtaacctactcatttcaacttttaggcagtgcagtaattacttactcatgttaatggtgaaatgcattaagataaacttcattcaacggattaaatctgggttcattataaatgtactcttagttactcctccatgtaactgcatttagcgaacaacaaaagctctaataaataatatgaaacacatgctaatttataaatgacatcatgggtatagatgctgtaacatcctaaacttttaaaataaataataaatcatatttgggatttaaagtaatattttaagataattgataaataagagaatgaaatacaaagaggcattttatttttagcctttgatttataaaaaagaaaaaaccttgaaattggatgtattttgaaatgggtaagtattttaaaagatgtgtaagttaagggggataaatggaaattaaggagataagcatttgaccaaagtcaaaatagaaggaacaataagtcggtcgattgttttcctgtgtgcatcttcggacgacagagagacagaaaagaacgagcgagagacagagaggaatcgagacagagagattatcgagagagagacagcgaagtcgggagagggagaccgagaacgaccgagacaggaggaaaaccgagagacaaggggaggacgaggcagcagcagcctaggctgctgcgtttttggcgacagaaaaccaccgccagcggcggtggtggtgagcgacggtggtggtggagccgagaaggggttgtggtggtgttagaagtgatggttcatctcaatttcttgataatttgcccttttggaacaataaattgaaggtaactttcaaagctttagatgattatctcttgaaagtagaaagcaagggtaatttagtcttcaattatgttatggagaagggtaataacacccaaatgctaacaaaatcatatagattaatgtgatttgaagttatatagttcatgaatgtgaaggttgactttctgcaatggtcaaacatggacatggacatgattatagcatattagcaatgtaataggaatggaatcaccaattccattgttgaaaatagaaaatttgatgtgtgcagaattttgtaaaatttcccaaaacaccaactttaaggctgcataagtcatgcatgaagaatgcaaatcagtccattctttcgcctaacatcaagtccttgcaacatagattacacaggaaaaagaaacagccaaatccgagttcgtatgaggattgtatgactgttcaacgtttgtcaaaagttgctgtcatgctgtcaaaccagaatttttctaagtctgggaaatatgcagaaaatgggtttgaccagtttttggagcttcataacttgatttatacatatccaaatcagttgattcttgagcctaaactgtagagaaagatgaggagaaggcataggaaaaagaatcaacacatttggatatcgtatacaacctgtgcaaagggttgaagtaggctcaaaacccatttttgacataattgtcaattatatgaattgtgggtaacttttgccgtaatgaaatgttttgacatataagtgtttatattgacttataaacatgttaagactataaacttgataaagtaacatgtcaaaacccgaaaatgcctatttaggcaaagtatgatgcttaattgagaaatcggtcaaatagcataaaaactaaaagtgttctaaatggtccaagaagactttatacataacttacacatcattagcacatgatgtgtagtacatgtgaggtttgaacacttttatacttggaaaattgtcaagtagtcaaaaatgggacaaaagggtaaaattgtccaaaatgagtatatggctaattaaagcaagttgagctactgaaatgaacttagataagtattataaggactataaaagtataatacttggttaactatgagttttgaaggtaatgtcaatttaaagtaagttaaccaaaagaaagtaagttgctaattttgggatatatgacccaaatgagttatgcattggtttttaggttgtgtaatacctataatgagatatgaattgattatgagacatgtattgatgataatacaaatgatgtaatgaaataggttcgttgcctatcgtggagcaaggagcatgcggattagtgtagctagtcttacctagctactaaggtgagtacgtgtggattgttttcccattttcgggtacatggaaaaataatatttttataatgaagtgcctaatgttttgaaagaaagaatgaatgaaaatacttgatgcttttgaaagaatgttttaaaagaaagtaatgttttgaaagaaaaaggtttgatgctttacgtttgaaaatgaatatgtcttgatattaatgaatggaatgggtaaagcatgaatgatataatatgtaataataagatgctaaggatggttatgtttatgatgttcacgtgaatataagaatgttgctcgatctaagatgtcccgggtagggattcagaggagcctatcggggtggtacctccccttcgcgagataggttacctaatgtaaatgatgtcaatgtaatctgatttgttctttttgcattggtacagacttggggtatatcagacccaagtataatatgatatggccatatgataagatgattaaaagagtatgtgaaatgttaaaggttatgtaaaatgttatatgaaattattcaaagagatctttatggttattatgtctatgaaaaggaagcatataatgttatagatattttatgtctaaacccacgtagctcaccagactagttgtctgacgtatttatttttatgccatgtatttcaggttatacacgaggatagactgatgatcgatagaagctagatgttacgacggatagacggagtggagctttcaagttctattgtaatgatcttaccgtaaattcttatttgctttgctatggttgctgtttgatatataattggtaacacccgagaattgttttatatatattcaaaaagtttttatttgggacggtttttgtaaacatgaccaagtgtcatgaaatatttttaaaaccatagcgtttttaaaacgataaaatgaggggtgttacagatgcgataatatatcttaggattatgtcaaatatttatgccaattatacatgagagagcaagtttataagagttttagttattctatcattggatcaatatagatacgtattaatagtagacagagatgcatgaatattatagcagagaactttttgcaaaacctaacctcactgcatgttgcattcatcaaccccacttgtagttgtaacaccatgaatatgccaattaagaaatatcatgaggtatattgtactggaaatgacaaaaagtactaatttacaatagaacgcagtcaaacttggttattttgtaaattttaagaagcaatttacaaaaaataaaacataataaagaaaaacataacagtaaatcacagacattgctcatggaagagtgactgatcaaactgaccgtttattttcttgtttcttgtacaactaccaaacgttcctgtataaccaccaaatagtcccagactttagaaaatagaacagaaaaccacagatatacatacatacagccatacattataatttttaattgaaaaaaatgcatagaaaaccagtctcaaataaagaaccaaataaagaagatacaaatgaaacctggttatcgaataaaagagttagagataatgatttcagcatttgcactcatgtaaacgatatgccattgggatccgttggaaagggtatcgtgaagtgattaaggagaagcggggtgttagggttttttttgcagtttattgaacaggttgatcaaattttgcgccggtagggttaattccttttcgaacgggtttctatctgggtctttcatcaaaatcactcatgtttgtgtctctaacctttagttgcagatggatatcagatcaacggtttcaggctaaatgggggaacaaataacaagaagaaagcggtgacatctgtgttcgtattcgtatggtggaaggggaagagtggcgtatgaattggtcaatgtaaaacatctggaaacaacttctcccaacatgtaaaaggatccggaaaatatgaaaatgacgattagagatttcgttcggatattgggaatatggtcgcgtctgattgccctacggtgcccaccgtttgggtttcttgatttgtcgtttagatagatgtcggaggtctaaattcacaatcctgttcttccgcggtaattgaaattgggtaaggctaattgtttagatcaaatggcagaggacgatgtgaagttgcgttgccctataattggctctcgtgaaattgaattgcaaatttcacaacgatgttagcaacaataatcgtcgctctcgcatcccataccgtcgcaacgtatgatggtgatcgagattgagagatttagggctagatgtggcgttggcggacataggttgtggttgacggaatgaggagaagacgtgagcgtcgattgaggtagcaacaggcggagggttaacggtgtcatcatggtgaaaggtcgcgcgtcgttttatggtcgatagatcatggacgttgtgagtgaatgccggcgcaaacacggtggaggaaagtctatgctttagttgacggggcggttttctctcggagacggaaagaggacgtcgaaagaaaggccggttggagaaaatatattaaacgcgccgacacacgctatcagagaagaccggttttcttatataataatgactaggtacgtgcaagggtcgtgctaagcacgacccatggaggcttcttgagaaaattgtaaacacatatcagataatagcgacgaactttactgttattgatatataaagtgattaagcatggttggccctaagttaaaactcaaactactgtaagtatgtaatggtaataatttatccttttcagctatcgatcttagattgctctgtagtgttgtgtcttagtgcttttcacggttcttttatatgcatatagtcacataaaatcagacacacacatagaataacccacagacaatcacataaaaatcagtctgatgaggtattaatttaagcaataaggattgatattcaagcaattagaggagaataatatattaatttcacgtactcctattgtagttattgtgacaaaacccaactgtttaattgtgccattgcaaaaagtccacctgtttaatttctttatcaacatgtcgttgcatccttttacatgcaaatatttcagaacatcttcctataatgacccttgagatgaactttttagaaatattgttgctggttacattatcagaaaaaacagagcttatgaaaaaagaccaaacatagtaactcgtacttcatgtctggtagttcttttgaatatttccgtcttttgttcgaaacaatttggttgaactgtagtagatctttcaaactgtatacaacttcacaatatgttttgtaagttcattacttctagatcctttcaaaagaagaggtcaaactcaaaatttgcataaaccagatttattgaaggctaattagattagtgttgtagggctttttggatataaataaaaatgaaacataggtgctgcaacaggtgcgaaagaatttggttggacagtagtaggtctttcaaactgtatacaactttacaatatgttttgtaagttcattatttctagatcctttcaaaaaaaaggaggtcaaactaaaaaaatttctaaaccagatttattgaaggctgattagattaacattgcagggctttttgggaataaatataaatgaaagataggtccaacagaaacgaaggaggtgtgaattatgcaacaaatggaaacacacaattatcgtagtacacatgtaattaacagggatcaatttccatttattgttccatcaattgccaaatttaaaagataacaaatgcatcctttttgaatggaaaagtatgatacataaaaccagagagaggtatacatttagtccagaaacggtggaaattactttacctagagttgcgtgaatcaaatggaaaatcagaggaagagaccctaaatcgcaatccatcttctattccctatagaatcaaatggaaatttcttacttccgacctttttcatttccacaagtttgtaatcgataagatcataaaagaaaaccgatttacattagcaaatctttaaggcacttgtttgtagttctcaagtatgcgttacgtatgaaattctagacagaaaagggacgacagggaaaataatggtggaataaataaacaatcaagaatcaaaagataataatccttaaaactaacaaaaattctatcaataagccctacaaatgagaaatatgacggtggagactcacctgattgcagtggcggaagctcttgggggctttggtaggccatggccccctgttttttggaaaattttcaaaaaaaaaaaaattatatatattatatcattgaataagtctagactcaggaaactatgaaagattagaaatagatcaagttatctaaatcaaatctcgaaaaatatggaaggaatcaaaacacaatatttatctcattgtgaaatcaaaaaccctataaaaaacacaaatctaacctacaatttaaatcaaaaacaccataaaaatcaacctaccgtcggattcagccgtcgtcgtcttcttcaccggaccaccatccaccgccattgcttcaatcgtgctttctcgtcaatagcttagtggcttgttcagctttcttgctctaccaatgatttttatacactttccagcaccaccgatttgttgaacggtaatattatgctcctttatatttttgaaatctttgaaagttcatctggttttctgtctctgtaaactgtaaagaaacttttatgttttgttaagaaaatagagagatagagaagaaagagtattgacgtgcttttcccatatgtctcgaatgccacattaggaaaagacaaaaagaaaacaattagtacattacgctagtgtgatagtactgtatgactattagtatgatttacagctttaatagggattcatagaaaagaacaaaataaactacttataaattaaaaatcgagctttatttttaattagtttaaataaatatttagctataacataatatattatatgttttgtatttataatcaaatataattatttgtattacagattagtttgtataatcaaatataatgattcatgtttcgaaaagattgtttaaatatttttatatgccatgttttttaactgaattatatttttttgtatgattttctttacaatttagtttttctttacaatttagttttggccccccttgtttttttgtttgtgttccgcccctgcctgattgatgcgagaaaaggaaagccgccgacatttgtgcttcttcccgcgttaagaaaaatcgcagatgacattacgattctttttctacagaggaataggggggttgcgactttgcgtgatttgttgttaatgagtgatgcgtcgtccgttttgatcggtagcccgtggagactgatttatttgacagatagatgtcgttctcgttgatgatgcaggtgtaattggcggcgttggtgaagcgaagatggcatagggaatggctttgcagaggagaaaaggagtttcacaaatatggattattgaatcgtcgtttcagagtcgcgatggttagtaatcgatggatttgggtttgcacctgttcaaggggtggtgacgtgtcttactgtaagaaagagtcgcgatggttagtaatcgacgatggatttgggtttgcaacatcgttgacggaaacgacgcggcgatggagagaggcggtagagggaatgaaacgggagatttaccttcaaccgcaaacatgtcttcgttcctcgaaaatcatatgcgccctttatgaaaattccttcacgcgccgtacacgcgctcagaaaaaacatagggttttcttatataataatgactaggaacgtttatgcgccgtgataatcacggcccatgagtggtacaaatgttgtaaaacttagaaaatgggaaagttgaatgtacaatgttatgatttattttattcttcatagcacaatacttttgctttagtgaaagcagttaaaaagtggatgtgtcgttagcaactataaacttttaacaaaatattacatacagaacatagaaataagataaacactaaaaaaatacaaaatagccgtgttcaacacggcccacagaatgcaatttggatatttctaaggaggtgtaaaaaatattgctattatgggagctaattctaggtatattttgttactcacatgacatgaatattgagtaattttgtaaaattggtaatttagcttagattttcccacaaagatgagtgttaaaactatcaaggaggatatatggtacttcctcaacaaagatgattttgaaatatctatattttaaatgtaagaactactatcacatgagacaaatattgatattactggttaggattacaacattgatatgtaaaatattttggtaatgtaaattttctaaaaaagcttggaagtagaatgcaacaacaatatatgctagtattacatgttgggtgattttatgaatgtacttacgtgttgatccttatttaccgtacttgaataattttatctgatcaatgctttacctatatggctatatctagcaatgtgcattattcacaaattatggagtgtaaattcatatagacgtatatgacagtggatgaaaaggaaaacaagtaatttgattgaatcactgagaaaaaagcaaaagatacatgcatataaaatgttacatcaaaatacagaagataaatgaaagaaagataataatgtggagcattcaaaataagtaagaatgtaagtccttgatagctcgaaattcttttaagtcttccgtgatagcctaatgtttataatagaagagacattcaaaatgaataagaattatatcacctaacagtatttaatcctactctacttggccattgagcatattgctattgttaaaaattatgtccaaatcgcatttaactgttagatgcaattctcattgccgattacacttatatgtatgtagtaaattataattaactctttgtcctggtcatttgatgtgtcatggatgtttgtaaggtacaaataatagattacaataacttaaatataaaattgagttaatattccagaattcacaaaatgtgtaataacacttatttataatttccacgtaattaatgattaactacttagaatcctacaagtttttgtttatcacctttcatatgatcgtataaatgcaacaacccttaatgcatttatagttgttggtctgccacgaacacctttttctgttgattatttgtcattattatgttaaaaaatgtttgaaaaatgttaaatgggattttgttatggcaaagagatgaacttctccaatattgtagttgcataaatattgccagttccatcatacagaataatacctcttatattgggcaatagaattatagaaacaataagggttaaccgacttacatgtattgatctctgattcttctttacacactggacaaattagtctatcatgtcttgcacaaccctgttaaagaaaacagtgaaaattgaccacgaaggtgtatcctctagtaccaacattacatctatcattttaaaagggaaatgtatacttatcatatatatctaatggacgttgcctttcctccaattgtcactcttacatctaatggaaattatgatcacaatttacttttaacagcatagatacattcatttgttggtaagacacttttggcggattcattgtattaatctttcatgtaaaaattctcattcaagatattaggtttttgttagaacccttgttatttgaacatgcaacctgaattctgattgttcccaacaccaacattaataatacctcttccactagaccatcattccaccttcaggcccttgtgttcattattataagcactattcggtttagttcttttatgagtcctatatttacatccttaaccattttaacaagtccatcatattgggaattttaatataaatacccaatgttattcatttttgtcgcttcagtagatattgaccatactaaagatgagtatttaatactcatacaacatgaaagaaaaaaatgaaaaactaacagtcacttatgtagccccaatctccatacaaatagcgctttaagaataaaccaatttcaggtgaaattgtagaaaggacataaatctgtacatgaaaaaacattaacagttacacataaattatttgcacagagataaaaagcacaatatgtgatgagaagatttaaaagtaggaaagtgaacaaaaataaccatcaaaatccaatgttagtaataaagaaaatgtacgagcgattcataacattcatccaaaacccatatgcgatgttgaatgcatatctggataaaagaatttaataaacaataagcgcctgtaaaatcaataaacaaaatcaataaaaatagacctaaaaatgcaaccgacttattgtttctatacaccaataaatgattttcacgaattttatccaaagaaagataaaaagtcataaaattgaacgggcttaccgatttgaatcgtggtctgtatctgccacgcagatcagataacaatcataatctacatcaaagatttcagaaaaatatgaaacttccgccattactaatttgatatgaatttctcagagcctaaaacaaacctaaattactaaaaatcaaacacaggtttttgacgtttaccttgatttgtgaaaataggcatttgggttttttcgtaggattttctatgaaagaagccatcaagtttatgacattgtaaattaaagcaaagtatgaatatatgaaggataggaaaagagatcggattgtgatgatccggagagaaaaatacacaaaccagagagaagcgatgttgtgattgtggtgtattaatctgtttttaaacctctaaccacttcttgtaagtccgtaatcagacgaaatgcgcttgaataataagtctttcacgccagtgtcgaggattaatgccgtcaagggtggtggggaggtatttataggaagaaatgcggtggaggtgaatggaggcgtcgatcaaacccaagaggaaacggtttgtcgaaatagatgcgttggtcattcgccataagtttgtgaaacggtgagtgtatccttggcgtcgtttgaaagtttgtgaaacggtggcgatgctccgttaaaggaccggcaacggatcgagcgatatgtaaatgggagggcagaaatattgcatacgcgccggacacgcgcttatggaagtgatagtatggaagtgataggattttcttatataataatgatataacttaattttaaataacatcgtactatacgtttcaaaataaattagagttttaataaaattacggaatgattaaaccaattacaaacaaatataagtgtcggtgatataaaatttgtaacaacgaaaacatttcaaaaaatctgaatgcggaaatccataacatgagaatacgattcgtttgcagtcacgccggtttcttcaccttctgagggtcctgaaaaaggttaataaacaatgggtaagctaaagtttagtgatcatacacattacttgtctattatatgagttagatacattcacatatcacatacatacaagcatacacatattacaaaaaatcctcacacatattgttggtaccaactactatttagtggaccaggttattcttgggggtcaattgtggttaaccataaatgtttggagtttaggttccccttgggggccgaccataaacacccatatcatatctctgtctaggttctctttggggactgactatagacaactttgctcagattctccttggaggataaccatcctcgacgagattaacaacggaccaccactgttttcagcttgtaacctgcaacatcaatagttgcgatcttcggcgacaatgatgaaccaacatcaacacaacagtagtgccggtatgttagtcgttggataatagaggcatcatctgacccttttagcggtgacttttgttgcctatgaaggtcttatcagttatatatcgattaggcttggttctaggttataagaaaaaatgattctctatccggtatgacatagagacaaaccggtctacttttataagaaaatcatatttgaaacaatgtcgtgttaataattaatatgtgtatgtttaaatttagaatatcctgaacttatatttagttcgcaaccctatgtaagatcgaactaattttaaaccgtaaccagacgtgcatgtttgatcagtaataaaaaggctatcatcgtcgaaaatattaaaacaaaatataatatcttatttggcgccatttc

SEQ ID NO: 8 - DNA, 684 bp, Taraxacum officinale
atgtcaacggttaaaagcaagttatcaacgcctccaaccgatcatttctccggtaaccaccaactgctactaataaccgccaccgcaaaaacctatatatacaacttccatcacaattctgtcccctactcccatttcaatccaaaaaaggctacacagacatatacagcgatggcagataatggcaacaccggccgtcaaaaggatgacgacggtggccatgatgggggacgcccaaatccgaccaccccaccctccccttcccgcaccccgcgaagacccaggcggaccacatcgccgcctaaacattctccgggggcgtcttcaagcaccatgccagcgccgcctactccccctgcgccgacgggaatcaccggtgcgtctagttcttctgtgggtactaatataatttcatttactccacccaaaaccaaaagaacgaagtcggtgatttgtccgatctgtaagaaagatatgtgccatgagaaggcgctgtgtggccacatccggtggcatactcaggaagaaagattggcggccagcattgctatagcaagagcgctatcttctaacgttgttgtttctggcaatggcgatgaagatgaaggtccatctaaaaagtataaactcccggacctgaacaagtcaccaccgccggaggaggaggacgaggacgctgcctga

SEQ ID NO: 9 - DNA, 4521 bp, Taraxacum officinale
ttttcttgcgggagaggcatgtatgtgtggcctttttgcaataatactatgcatgggagatgcataaagtttatctttattttgttttagtttaataatgtgtggcaaactttatatcatttatgatttcggccatgtactgctacatcgttctaggatggttattccctagaatttgcttttatatattttttttcttcgttaattcttctcttcctttaacgttatttcatgtgaatttgcgatatcatgatattgcaggaacgtaacttagaactcaacgataaaaagtgtgttgagatcgtggaagttgatcgtttatcttgaattcacaagataagcaacatgagatagaataattaattactgcggagatttctttttagattcatcttattcgttgtaaattgcaatgcaaatttttaagtagaataacgaaagcgtgttgttatttacggttaaatgcaaaaaatcattaacatatctccataattgatttgtttagtatgcattctcatgtgttctattgcaaaattctattttgaaattcaattaaagcaatgcattttagatataaagcaattccatcacttaattatattgggtagatttttaccaaatcactgtcctaaaagaaaaataaaaaatgaaagttcaatgccatagtgatcccgtcaattatgtgatcaatttttaccagagcaaaaccccaaaagttaaagagtataattgcagaacgacaacaatctgaggatgatactaactccggtataaaccgaatgcttttgtggggatttcactaatcaactatttggctctcatcacaaggccgtgttaatggattactattagttataaggccatcccaagttgtttggctatattattaccttttaaaaaccaaaatgtgtaaactgtaaaattacacttttggttcttgtattttctaatttttagaattatgtatttggttcttgtagtttttttatttgtatgaatcatattagttttggtccacgataacttttattttctataattacacttttggtcattgtagttttaatttgcaagaaaaaaataatctatctttttagtccatatatatagtttgcaatattctatattttaggttcaggttatatatcttttacaaaaatactacttatgattttttaatgaatatatcatatttcgtaaattacataaacattttatataattttatttagtcagaactataggattcgcatgataactttgctgaacttaattgggcttcatttttaacaaaggataatgcatcatgcatgacataaaaatttgtaatgtttctaatgtgcatgcactacaagaacctgacccttcatatacattaatgaatgattctcttttcttattatatcttagcaaacggtttggataagagtctgaacaatattaagaattgcactagctagctagtccatgcagcaagcaaattaattagaggactttttaagaggcttgatcacttcattgtatatctaatggcactgtggatatatgattggattgtgtgtatgtatggaatttaatattactaaaaaatattaatgacaaacaataacacactaatttataacttacaaaagcgtgtgtcattaaagaaaatgtcatgtttcttaaaatatggagaatttactacttacataattatgtgtcataaatttatgtcatcttgtgtgatatttatgacttacaaaaatgtatgttatgtttatcgtcataaaatctgtaaaaaaaaatagaaacatggaggatactttcaaggtgtagaaacttgacagcataaacgggtttattaccttcatcaaaacttatcttacttcaatctcttggagatgcaggttgattttaatgctaaatgaacttttcaaatgtataaatatatatcttttgacggatttgacattgatggagatgaagatacaccagtgaattcacctttcaatgatcatactgacataccaagtactcttatcccctctccacctccacagaacgaacctcaatttcaaaaacgtaactgccgctgttctaaaaacagaaaattttcaaaaacgtaactatcatttaaatatttaatcatttttcaattggattcatgctgtaatagaagttactggactttaaacttcttgtagctgatgagattcatgaacaacagtccaaggagattttccaaatcaacaagtttctgatgagaacaaggtaaaaaaaataagtttttcatttaaacatatgattttggttttcaaaagataattaattataggaactcttctgaaaagtcccaaagtttacccgtaaaattgattttggtttttttttaaattgaataaaaaagtccataaaattggataaattattcgatttagcccaatttacctagtatatctggtaaaaattggttaaatcgaataatttaggcagtttgttgggcttttttgcgcaaaaacattattttgggacttaatcgttaattttccccaagtttcagtttatttacagtattggaataaaaccgaacctagtgcgctaattttgtcttttggtgtttagaattatttgggtttattggtctcggtttctaaacgagttcggtttttggtttcttgattcgatttttggcttttgggttcaacccatggattttcgatttggttttggttttgataatattttttgtgcaatcgttcaaaaaatccgagttagattgggtttaaatgttatcaaaaccaaaaccgatggtagaaccccaaaactgaacccaatcgaaaatccatgggttgaacctcaaaaacgaacccaataaattctaaaaaccaaaatccaaaattagcggataaaaattttaaaatacatatctactggtaaggtgtttttgacaatttatgtaaattatagaaaaaaatgtattaagtgtttgctatttaagataaaaatggcgttaaaaatagaattggttagaaatgaaccatttttttatattttttacttcatttattaagtaaatagtagaggtgaattagagatgatctaagatattttttttctttcaaaaagagcttcgaaaacacttttccgacagaaacaagacacaaacctttttactcgtagaccgacgatttttttaaagatcaaagacctaagaagatttgtctcccatcttcttaaaacatatgtgagatagacacttctagcctcttctatgctaaaaaaaagagctcgcctttagtttggttaatttgagacaatggtctccacaaaaacataggtagttaggtactcacgcgccaagttactaatctcaaatcaaacataactgtatgatcgtcaacacattttacaagttaacaaaagagcattgctttagacttaaaaattgctttgagaaaaaacaaacacctaagaatttctaaaagcgatgatttcaactctctttaaaatgtatatatatgttacattttggtctttaaacttttcaagaaagtacaattcaactcctattttcaaaatgttcttacatgagcgatatgattttaccgtttaaaatgtaaaccatgataactatttctaaattccaatttttgtttcgttgatcaatctcattatcattcaatgcatacaaaacacaatacaatctgttttgtcatcttcttcaaggccgaaagacaactaaacatatgaagactgaagaggacaatctagcaaactcaaacttcactataaatttgcaattgctattggtagccatgaagtaacatttttgaggaggttaacaaaattttaacagtaaattcaataatccatcaccacgatatgtaatcatctacacaaacacagatgatgatagtcatatatgttcgtaatcacacaagcaagaaatcgcaatgtactttacaccgtcttctctgacttgaacacgggaattggttgtctaagaagtagattgtttcccacggatttaccaagtacatttaaatccttcaccctttgtatctctcttcctgttttttgttttgcaatcaacaaaaaaaaaagtaaaaatcccatcttaaattcacgcaaaaaaacacattgaatcaaccatcactatagtagtttcaagaaaaatctgcacttccataatttctttctattacaccattatactaggaaaacttcatagcaatattatctaaatacaaagcaatttttactagtaatattcgtagatttgtcaatatatagtgtcctaataaagaaaaaataacggaactataatgctctaataggtagattttttttagtataatgtcctaataagagaaaaaacaaatgtacaattatgtaataataagaaaagaaagtaatatgcttaatagacaaataaatgaaaccatgttgctatttcttgcatctagtccttatttgaataatatgtagcatattaactacttatgaattttatatataaatgataaaaaattccgtgag

SEQ ID NO: 10 - DNA, 19724 bp, Taraxacum officinale
ctgacttggccaacattttttgttcttccgatcccttcgtaacaatcagtttaagcacaattgaattatatggagataaataggtgaaatttggaagacgaggatgggagagtgcgaagagccaaagaagagaaatgtgggatagcggccggccggttgttaatagaagtgcttagatcttggagaccttctaatgcttatggaggaaccagatgcttcaatttgtaaatctgcgctgcccacgtagaatattgaaaaagaaaatcaaatcgatcaataatgcgaccatatactttaggatttcccacttgtataattctcttattcattttcaaatctttatctaaattaaggatatcggcaaaaatataatcctaattgtttttcaaaatgataaggattataaggtaacttaattcttgttttattcatatttaattattattgttttttatcaatgttttaaaacccgggttttgagtcaacccggtcttgtgaaaaatcccgggtcagccggtcagctgatctaccggttcaatatatttaaataaaatataaattttcatatagaatttgagttttaggctaagaaaatcgggttttgggctaaggaaaatcgggtttaagttttgggctaatggattttcgggtcaataacgtttttttccgttttttagatcggttcgaccgcgttttgtgtgaaacccggccgggttgatccgagtcaataattggcaacaacccggtatatgttgacccgctcttgtccccgggtcccggttcaaccggttggatcggccgggttgaccctggttttaaaacactgttttttatacaatatatttgtttttgtttttagttttgtttttgtttttgattttgtagatatatttttaattttatttttattttgtataaatatagtcattacaaccgattatttgtaagttgagtcggtcaaaacgaactttctgataattcttatacatttttcggcgaaacatgaatattttgatcaaccaatatgcttttttctaggttttttaaataaaccgcacgttcaattgcatattcgcaaacaatagaactcgaaaaccctaatcgagcttgttttatagaaatctgggttgagtgcatacataataattaataactgtgaaaaagtttgttttaacatgctaaacctatcgatagtcggttataataattatatgtaaacaaaatcagaaacaataatgatatatgtcacccaaaaaaatagttactaaatatatacaaaatcaaaaacaacagtaatttcttttacaaaaaaaagagaacaatagtgactaactttagaaagaaaaaaaaagaactaaattactcttataaatcattatcttttatataaattggtaaattggtcatgtaatctagaatgtgaattgcggaatgctttagtcacattttgaacaaactggtaattattaatttcgataaaagttcttcaagaatagtttggtcacattttggacgacaatggtgtgttttccatagtaagcaatacgtaatgagttcgcttaataggaaaatagatctaaagcttaatgaaatcatatgtataagtaacatatatttttcacacgttacattgtttgtaacgtcctcaatttttttaatatatataactaggtacactctaaggtcgtgctaagcacgacccacgaatggtaatttaggtaatttttcatccttgactaaaatacattatgctaataacaaagaattaagaatacaatgctataatgcataagtaaataaaagtacatgatatttctcacatttagtctttatacatagaaaacttacactggccatttgcaaaaaaacattaaatctccctaaaaagtagagtttacaaaatgaaagcttgtttttatagcaaacaataatcaatatgtgaagctatagtatagtaacctactcatttcaacttttaggcagtgcagtaattacttactcatgttaatggtgaaatgcattaagataaacttcattcaacggattaaatctgggttcattataaatgtactcttagttactcctccatgtaactgcatttagcgaacaacaaaagctctaataaataatatgaaacacatgctaatttataaatgacatcatgggtatagatgctgtaacatcctaaacttttaaaataaataataaatcatatttgggatttaaagtaatattttaagataattgataaataagagaatgaaatacaaagaggcattttatttttagcctttgatttataaaaaagaaaaaaccttgaaattggatgtattttgaaatgggtaagtattttaaaagatgtgtaagttaagggggataaatggaaattaaggagataagcatttgaccaaagtcaaaatagaaggaacaataagtcggtcgattgttttcctgtgtgcatcttcggacgacagagagacagaaaagaacgagcgagagacagagaggaatcgagacagagagattatcgagagagagacagcgaagtcgggagagggagaccgagaacgaccgagacaggaggaaaaccgagagacaaggggaggacgaggcagcagcagcctaggctgctgcgtttttggcgacagaaaaccaccgccagcggcggtggtggtgagcgacggtggtggtggagccgagaaggggttgtggtggtgttagaagtgatggttcatctcaatttcttgataatttgcccttttggaacaataaattgaaggtaactttcaaagctttagatgattatctcttgaaagtagaaagcaagggtaatttagtcttcaattatgttatggagaagggtaataacacccaaatgctaacaaaatcatatagattaatgtgatttgaagttatatagttcatgaatgtgaaggttgactttctgcaatggtcaaacatggacatggacatgattatagcatattagcaatgtaataggaatggaatcaccaattccattgttgaaaatagaaaatttgatgtgtgcagaattttgtaaaatttcccaaaacaccaactttaaggctgcataagtcatgcatgaagaatgcaaatcagtccattctttcgcctaacatcaagtccttgcaacatagattacacaggaaaaagaaacagccaaatccgagttcgtatgaggattgtatgactgttcaacgtttgtcaaaagttgctgtcatgctgtcaaaccagaatttttctaagtctgggaaatatgcagaaaatgggtttgaccagtttttggagcttcataacttgatttatacatatccaaatcagttgattcttgagcctaaactgtagagaaagatgaggagaaggcataggaaaaagaatcaacacatttggatatcgtatacaacctgtgcaaagggttgaagtaggctcaaaacccatttttgacataattgtcaattatatgaattgtgggtaacttttgccgtaatgaaatgttttgacatataagtgtttatattgacttataaacatgttaagactataaacttgataaagtaacatgtcaaaacccgaaaatgcctatttaggcaaagtatgatgcttaattgagaaatcggtcaaatagcataaaaactaaaagtgttctaaatggtccaagaagactttatacataacttacacatcattagcacatgatgtgtagtacatgtgaggtttgaacacttttatacttggaaaattgtcaagtagtcaaaaatgggacaaaagggtaaaattgtccaaaatgagtatatggctaattaaagcaagttgagctactgaaatgaacttagataagtattataaggactataaaagtataatacttggttaactatgagttttgaaggtaatgtcaatttaaagtaagttaaccaaaagaaagtaagttgctaattttgggatatatgacccaaatgagttatgcattggtttttaggttgtgtaatacctataatgagatatgaattgattatgagacatgtattgatgataatacaaatgatgtaatgaaataggttcgttgcctatcgtggagcaaggagcatgcggattagtgtagctagtcttacctagctactaaggtgagtacgtgtggattgttttcccattttcgggtacatggaaaaataatatttttataatgaagtgcctaatgttttgaaagaaagaatgaatgaaaatacttgatgcttttgaaagaatgttttaaaagaaagtaatgttttgaaagaaaaaggtttgatgctttacgtttgaaaatgaatatgtcttgatattaatgaatggaatgggtaaagcatgaatgatataatatgtaataataagatgctaaggatggttatgtttatgatgttcacgtgaatataagaatgttgctcgatctaagatgtcccgggtagggattcagaggagcctatcggggtggtacctccccttcgcgagataggttacctaatgtaaatgatgtcaatgtaatctgatttgttctttttgcattggtacagacttggggtatatcagacccaagtataatatgatatggccatatgataagatgattaaaagagtatgtgaaatgttaaaggttatgtaaaatgttatatgaaattattcaaagagatctttatggttattatgtctatgaaaaggaagcatataatgttatagatattttatgtctaaacccacgtagctcaccagactagttgtctgacgtatttatttttatgccatgtatttcaggttatacacgaggatagactgatgatcgatagaagctagatgttacgacggatagacggagtggagctttcaagttctattgtaatgatcttaccgtaaattcttatttgctttgctatggttgctgtttgatatataattggtaacacccgagaattgttttatatatattcaaaaagtttttatttgggacggtttttgtaaacatgaccaagtgtcatgaaatatttttaaaaccatagcgtttttaaaacgataaaatgaggggtgttacagatgcgataatatatcttaggattatgtcaaatatttatgccaattatacatgagagagcaagtttataagagttttagttattctatcattggatcaatatagatacgtattaatagtagacagagatgcatgaatattatagcagagaactttttgcaaaacctaacctcactgcatgttgcattcatcaaccccacttgtagttgtaacaccatgaatatgccaattaagaaatatcatgaggtatattgtactggaaatgacaaaaagtactaatttacaatagaacgcagtcaaacttggttattttgtaaattttaagaagcaatttacaaaaaataaaacataataaagaaaaacataacagtaaatcacagacattgctcatggaagagtgactgatcaaactgaccgtttattttcttgtttcttgtacaactaccaaacgttcctgtataaccaccaaatagtcccagactttagaaaatagaacagaaaaccacagatatacatacatacagccatacattataatttttaattgaaaaaaatgcatagaaaaccagtctcaaataaagaaccaaataaagaagatacaaatgaaacctggttatcgaataaaagagttagagataatgatttcagcatttgcactcatgtaaacgatatgccattgggatccgttggaaagggtatcgtgaagtgattaaggagaagcggggtgttagggttttttttgcagtttattgaacaggttgatcaaattttgcgccggtagggttaattccttttcgaacgggtttctatctgggtctttcatcaaaatcactcatgtttgtgtctctaacctttagttgcagatggatatcagatcaacggtttcaggctaaatgggggaacaaataacaagaagaaagcggtgacatctgtgttcgtattcgtatggtggaaggggaagagtggcgtatgaattggtcaatgtaaaacatctggaaacaacttctcccaacatgtaaaaggatccggaaaatatgaaaatgacgattagagatttcgttcggatattgggaatatggtcgcgtctgattgccctacggtgcccaccgtttgggtttcttgatttgtcgtttagatagatgtcggaggtctaaattcacaatcctgttcttccgcggtaattgaaattgggtaaggctaattgtttagatcaaatggcagaggacgatgtgaagttgcgttgccctataattggctctcgtgaaattgaattgcaaatttcacaacgatgttagcaacaataatcgtcgctctcgcatcccataccgtcgcaacgtatgatggtgatcgagattgagagatttagggctagatgtggcgttggcggacataggttgtggttgacggaatgaggagaagacgtgagcgtcgattgaggtagcaacaggcggagggttaacggtgtcatcatggtgaaaggtcgcgcgtcgttttatggtcgatagatcatggacgttgtgagtgaatgccggcgcaaacacggtggaggaaagtctatgctttagttgacggggcggttttctctcggagacggaaagaggacgtcgaaagaaaggccggttggagaaaatatattaaacgcgccgacacacgctatcagagaagaccggttttcttatataataatgactaggtacgtgcaagggtcgtgctaagcacgacccatggaggcttcttgagaaaattgtaaacacatatcagataatagcgacgaactttactgttattgatatataaagtgattaagcatggttggccctaagttaaaactcaaactactgtaagtatgtaatggtaataatttatccttttcagctatcgatcttagattgctctgtagtgttgtgtcttagtgcttttcacggttcttttatatgcatatagtcacataaaatcagacacacacatagaataacccacagacaatcacataaaaatcagtctgatgaggtattaatttaagcaataaggattgatattcaagcaattagaggagaataatatattaatttcacgtactcctattgtagttattgtgacaaaacccaactgtttaattgtgccattgcaaaaagtccacctgtttaatttctttatcaacatgtcgttgcatccttttacatgcaaatatttcagaacatcttcctataatgacccttgagatgaactttttagaaatattgttgctggttacattatcagaaaaaacagagcttatgaaaaaagaccaaacatagtaactcgtacttcatgtctggtagttcttttgaatatttccgtcttttgttcgaaacaatttggttgaactgtagtagatctttcaaactgtatacaacttcacaatatgttttgtaagttcattacttctagatcctttcaaaagaagaggtcaaactcaaaatttgcataaaccagatttattgaaggctaattagattagtgttgtagggctttttggatataaataaaaatgaaacataggtgctgcaacaggtgcgaaagaatttggttggacagtagtaggtctttcaaactgtatacaactttacaatatgttttgtaagttcattatttctagatcctttcaaaaaaaaggaggtcaaactaaaaaaatttctaaaccagatttattgaaggctgattagattaacattgcagggctttttgggaataaatataaatgaaagataggtccaacagaaacgaaggaggtgtgaattatgcaacaaatggaaacacacaattatcgtagtacacatgtaattaacagggatcaatttccatttattgttccatcaattgccaaatttaaaagataacaaatgcatcctttttgaatggaaaagtatgatacataaaaccagagagaggtatacatttagtccagaaacggtggaaattactttacctagagttgcgtgaatcaaatggaaaatcagaggaagagaccctaaatcgcaatccatcttctattccctatagaatcaaatggaaatttcttacttccgacctttttcatttccacaagtttgtaatcgataagatcataaaagaaaaccgatttacattagcaaatctttaaggcacttgtttgtagttctcaagtatgcgttacgtatgaaattctagacagaaaagggacgacagggaaaataatggtggaataaataaacaatcaagaatcaaaagataataatccttaaaactaacaaaaattctatcaataagccctacaaatgagaaatatgacggtggagactcacctgattgcagtggcggaagctcttgggggctttggtaggccatggccccctgttttttggaaaattttcaaaaaaaaaaaaattatatatattatatcattgaataagtctagactcaggaaactatgaaagattagaaatagatcaagttatctaaatcaaatctcgaaaaatatggaaggaatcaaaacacaatatttatctcattgtgaaatcaaaaaccctataaaaaacacaaatctaacctacaatttaaatcaaaaacaccataaaaatcaacctaccgtcggattcagccgtcgtcgtcttcttcaccggaccaccatccaccgccattgcttcaatcgtgctttctcgtcaatagcttagtggcttgttcagctttcttgctctaccaatgatttttatacactttccagcaccaccgatttgttgaacggtaatattatgctcctttatatttttgaaatctttgaaagttcatctggttttctgtctctgtaaactgtaaagaaacttttatgttttgttaagaaaatagagagatagagaagaaagagtattgacgtgcttttcccatatgtctcgaatgccacattaggaaaagacaaaaagaaaacaattagtacattacgctagtgtgatagtactgtatgactattagtatgatttacagctttaatagggattcatagaaaagaacaaaataaactacttataaattaaaaatcgagctttatttttaattagtttaaataaatatttagctataacataatatattatatgttttgtatttataatcaaatataattatttgtattacagattagtttgtataatcaaatataatgattcatgtttcgaaaagattgtttaaatatttttatatgccatgttttttaactgaattatatttttttgtatgattttctttacaatttagtttttctttacaatttagttttggccccccttgtttttttgtttgtgttccgcccctgcctgattgatgcgagaaaaggaaagccgccgacatttgtgcttcttcccgcgttaagaaaaatcgcagatgacattacgattctttttctacagaggaataggggggttgcgactttgcgtgatttgttgttaatgagtgatgcgtcgtccgttttgatcggtagcccgtggagactgatttatttgacagatagatgtcgttctcgttgatgatgcaggtgtaattggcggcgttggtgaagcgaagatggcatagggaatggctttgcagaggagaaaaggagtttcacaaatatggattattgaatcgtcgtttcagagtcgcgatggttagtaatcgatggatttgggtttgcacctgttcaaggggtggtgacgtgtcttactgtaagaaagagtcgcgatggttagtaatcgacgatggatttgggtttgcaacatcgttgacggaaacgacgcggcgatggagagaggcggtagagggaatgaaacgggagatttaccttcaaccgcaaacatgtcttcgttcctcgaaaatcatatgcgccctttatgaaaattccttcacgcgccgtacacgcgctcagaaaaaacatagggttttcttatataataatgactaggaacgtttatgcgccgtgataatcacggcccatgagtggtacaaatgttgtaaaacttagaaaatgggaaagttgaatgtacaatgttatgatttattttattcttcatagcacaatacttttgctttagtgaaagcagttaaaaagtggatgtgtcgttagcaactataaacttttaacaaaatattacatacagaacatagaaataagataaacactaaaaaaatacaaaatagccgtgttcaacacggcccacagaatgcaatttggatatttctaaggaggtgtaaaaaatattgctattatgggagctaattctaggtatattttgttactcacatgacatgaatattgagtaattttgtaaaattggtaatttagcttagattttcccacaaagatgagtgttaaaactatcaaggaggatatatggtacttcctcaacaaagatgattttgaaatatctatattttaaatgtaagaactactatcacatgagacaaatattgatattactggttaggattacaacattgatatgtaaaatattttggtaatgtaaattttctaaaaaagcttggaagtagaatgcaacaacaatatatgctagtattacatgttgggtgattttatgaatgtacttacgtgttgatccttatttaccgtacttgaataattttatctgatcaatgctttacctatatggctatatctagcaatgtgcattattcacaaattatggagtgtaaattcatatagacgtatatgacagtggatgaaaaggaaaacaagtaatttgattgaatcactgagaaaaaagcaaaagatacatgcatataaaatgttacatcaaaatacagaagataaatgaaagaaagataataatgtggagcattcaaaataagtaagaatgtaagtccttgatagctcgaaattcttttaagtcttccgtgatagcctaatgtttataatagaagagacattcaaaatgaataagaattatatcacctaacagtatttaatcctactctacttggccattgagcatattgctattgttaaaaattatgtccaaatcgcatttaactgttagatgcaattctcattgccgattacacttatatgtatgtagtaaattataattaactctttgtcctggtcatttgatgtgtcatggatgtttgtaaggtacaaataatagattacaataacttaaatataaaattgagttaatattccagaattcacaaaatgtgtaataacacttatttataatttccacgtaattaatgattaactacttagaatcctacaagtttttgtttatcacctttcatatgatcgtataaatgcaacaacccttaatgcatttatagttgttggtctgccacgaacacctttttctgttgattatttgtcattattatgttaaaaaatgtttgaaaaatgttaaatgggattttgttatggcaaagagatgaacttctccaatattgtagttgcataaatattgccagttccatcatacagaataatacctcttatattgggcaatagaattatagaaacaataagggttaaccgacttacatgtattgatctctgattcttctttacacactggacaaattagtctatcatgtcttgcacaaccctgttaaagaaaacagtgaaaattgaccacgaaggtgtatcctctagtaccaacattacatctatcattttaaaagggaaatgtatacttatcatatatatctaatggacgttgcctttcctccaattgtcactcttacatctaatggaaattatgatcacaatttacttttaacagcatagatacattcatttgttggtaagacacttttggcggattcattgtattaatctttcatgtaaaaattctcattcaagatattaggtttttgttagaacccttgttatttgaacatgcaacctgaattctgattgttcccaacaccaacattaataatacctcttccactagaccatcattccaccttcaggcccttgtgttcattattataagcactattcggtttagttcttttatgagtcctatatttacatccttaaccattttaacaagtccatcatattgggaattttaatataaatacccaatgttattcatttttgtcgcttcagtagatattgaccatactaaagatgagtatttaatactcatacaacatgaaagaaaaaaatgaaaaactaacagtcacttatgtagccccaatctccatacaaatagcgctttaagaataaaccaatttcaggtgaaattgtagaaaggacataaatctgtacatgaaaaaacattaacagttacacataaattatttgcacagagataaaaagcacaatatgtgatgagaagatttaaaagtaggaaagtgaacaaaaataaccatcaaaatccaatgttagtaataaagaaaatgtacgagcgattcataacattcatccaaaacccatatgcgatgttgaatgcatatctggataaaagaatttaataaacaataagcgcctgtaaaatcaataaacaaaatcaataaaaatagacctaaaaatgcaaccgacttattgtttctatacaccaataaatgattttcacgaattttatccaaagaaagataaaaagtcataaaattgaacgggcttaccgatttgaatcgtggtctgtatctgccacgcagatcagataacaatcataatctacatcaaagatttcagaaaaatatgaaacttccgccattactaatttgatatgaatttctcagagcctaaaacaaacctaaattactaaaaatcaaacacaggtttttgacgtttaccttgatttgtgaaaataggcatttgggttttttcgtaggattttctatgaaagaagccatcaagtttatgacattgtaaattaaagcaaagtatgaatatatgaaggataggaaaagagatcggattgtgatgatccggagagaaaaatacacaaaccagagagaagcgatgttgtgattgtggtgtattaatctgtttttaaacctctaaccacttcttgtaagtccgtaatcagacgaaatgcgcttgaataataagtctttcacgccagtgtcgaggattaatgccgtcaagggtggtggggaggtatttataggaagaaatgcggtggaggtgaatggaggcgtcgatcaaacccaagaggaaacggtttgtcgaaatagatgcgttggtcattcgccataagtttgtgaaacggtgagtgtatccttggcgtcgtttgaaagtttgtgaaacggtggcgatgctccgttaaaggaccggcaacggatcgagcgatatgtaaatgggagggcagaaatattgcatacgcgccggacacgcgcttatggaagtgatagtatggaagtgataggattttcttatataataatgatataacttaattttaaataacatcgtactatacgtttcaaaataaattagagttttaataaaattacggaatgattaaaccaattacaaacaaatataagtgtcggtgatataaaatttgtaacaacgaaaacatttcaaaaaatctgaatgcggaaatccataacatgagaatacgattcgtttgcagtcacgccggtttcttcaccttctgagggtcctgaaaaaggttaataaacaatgggtaagctaaagtttagtgatcatacacattacttgtctattatatgagttagatacattcacatatcacatacatacaagcatacacatattacaaaaaatcctcacacatattgttggtaccaactactatttagtggaccaggttattcttgggggtcaattgtggttaaccataaatgtttggagtttaggttccccttgggggccgaccataaacacccatatcatatctctgtctaggttctctttggggactgactatagacaactttgctcagattctccttggaggataaccatcctcgacgagattaacaacggaccaccactgttttcagcttgtaacctgcaacatcaatagttgcgatcttcggcgacaatgatgaaccaacatcaacacaacagtagtgccggtatgttagtcgttggataatagaggcatcatctgacccttttagcggtgacttttgttgcctatgaaggtcttatcagttatatatcgattaggcttggttctaggttataagaaaaaatgattctctatccggtatgacatagagacaaaccggtctacttttataagaaaatcatatttgaaacaatgtcgtgttaataattaatatgtgtatgtttaaatttagaatatcctgaacttatatttagttcgcaaccctatgtaagatcgaactaattttaaaccgtaaccagacgtgcatgtttgatcagtaataaaaaggctatcatcgtcgaaaatattaaaacaaaatataatatcttatttggcgccatttcatgtcaacggttaaaagcaagttatcaacgcctccaaccgatcatttctccggtaaccaccaactgctactaataaccgccaccgcaaaaacctatatatacaacttccatcacaattctgtcccctactcccatttcaatccaaaaaaggctacacagacatatacagcgatggcagataatggcaacaccggccgtcaaaaggatgacgacggtggccatgatgggggacgcccaaatccgaccaccccaccctccccttcccgcaccccgcgaagacccaggcggaccacatcgccgcctaaacattctccgggggcgtcttcaagcaccatgccagcgccgcctactccccctgcgccgacgggaatcaccggtgcgtctagttcttctgtgggtactaatataatttcatttactccacccaaaaccaaaagaacgaagtcggtgatttgtccgatctgtaagaaagatatgtgccatgagaaggcgctgtgtggccacatccggtggcatactcaggaagaaagattggcggccagcattgctatagcaagagcgctatcttctaacgttgttgtttctggcaatggcgatgaagatgaaggtccatctaaaaagtataaactcccggacctgaacaagtcaccaccgccggaggaggaggacgaggacgctgcctgattttcttgcgggagaggcatgtatgtgtggcctttttgcaataatactatgcatgggagatgcataaagtttatctttattttgttttagtttaataatgtgtggcaaactttatatcatttatgatttcggccatgtactgctacatcgttctaggatggttattccctagaatttgcttttatatattttttttcttcgttaattcttctcttcctttaacgttatttcatgtgaatttgcgatatcatgatattgcaggaacgtaacttagaactcaacgataaaaagtgtgttgagatcgtggaagttgatcgtttatcttgaattcacaagataagcaacatgagatagaataattaattactgcggagatttctttttagattcatcttattcgttgtaaattgcaatgcaaatttttaagtagaataacgaaagcgtgttgttatttacggttaaatgcaaaaaatcattaacatatctccataattgatttgtttagtatgcattctcatgtgttctattgcaaaattctattttgaaattcaattaaagcaatgcattttagatataaagcaattccatcacttaattatattgggtagatttttaccaaatcactgtcctaaaagaaaaataaaaaatgaaagttcaatgccatagtgatcccgtcaattatgtgatcaatttttaccagagcaaaaccccaaaagttaaagagtataattgcagaacgacaacaatctgaggatgatactaactccggtataaaccgaatgcttttgtggggatttcactaatcaactatttggctctcatcacaaggccgtgttaatggattactattagttataaggccatcccaagttgtttggctatattattaccttttaaaaaccaaaatgtgtaaactgtaaaattacacttttggttcttgtattttctaatttttagaattatgtatttggttcttgtagtttttttatttgtatgaatcatattagttttggtccacgataacttttattttctataattacacttttggtcattgtagttttaatttgcaagaaaaaaataatctatctttttagtccatatatatagtttgcaatattctatattttaggttcaggttatatatcttttacaaaaatactacttatgattttttaatgaatatatcatatttcgtaaattacataaacattttatataattttatttagtcagaactataggattcgcatgataactttgctgaacttaattgggcttcatttttaacaaaggataatgcatcatgcatgacataaaaatttgtaatgtttctaatgtgcatgcactacaagaacctgacccttcatatacattaatgaatgattctcttttcttattatatcttagcaaacggtttggataagagtctgaacaatattaagaattgcactagctagctagtccatgcagcaagcaaattaattagaggactttttaagaggcttgatcacttcattgtatatctaatggcactgtggatatatgattggattgtgtgtatgtatggaatttaatattactaaaaaatattaatgacaaacaataacacactaatttataacttacaaaagcgtgtgtcattaaagaaaatgtcatgtttcttaaaatatggagaatttactacttacataattatgtgtcataaatttatgtcatcttgtgtgatatttatgacttacaaaaatgtatgttatgtttatcgtcataaaatctgtaaaaaaaaatagaaacatggaggatactttcaaggtgtagaaacttgacagcataaacgggtttattaccttcatcaaaacttatcttacttcaatctcttggagatgcaggttgattttaatgctaaatgaacttttcaaatgtataaatatatatcttttgacggatttgacattgatggagatgaagatacaccagtgaattcacctttcaatgatcatactgacataccaagtactcttatcccctctccacctccacagaacgaacctcaatttcaaaaacgtaactgccgctgttctaaaaacagaaaattttcaaaaacgtaactatcatttaaatatttaatcatttttcaattggattcatgctgtaatagaagttactggactttaaacttcttgtagctgatgagattcatgaacaacagtccaaggagattttccaaatcaacaagtttctgatgagaacaaggtaaaaaaaataagtttttcatttaaacatatgattttggttttcaaaagataattaattataggaactcttctgaaaagtcccaaagtttacccgtaaaattgattttggtttttttttaaattgaataaaaaagtccataaaattggataaattattcgatttagcccaatttacctagtatatctggtaaaaattggttaaatcgaataatttaggcagtttgttgggcttttttgcgcaaaaacattattttgggacttaatcgttaattttccccaagtttcagtttatttacagtattggaataaaaccgaacctagtgcgctaattttgtcttttggtgtttagaattatttgggtttattggtctcggtttctaaacgagttcggtttttggtttcttgattcgatttttggcttttgggttcaacccatggattttcgatttggttttggttttgataatattttttgtgcaatcgttcaaaaaatccgagttagattgggtttaaatgttatcaaaaccaaaaccgatggtagaaccccaaaactgaacccaatcgaaaatccatgggttgaacctcaaaaacgaacccaataaattctaaaaaccaaaatccaaaattagcggataaaaattttaaaatacatatctactggtaaggtgtttttgacaatttatgtaaattatagaaaaaaatgtattaagtgtttgctatttaagataaaaatggcgttaaaaatagaattggttagaaatgaaccatttttttatattttttacttcatttattaagtaaatagtagaggtgaattagagatgatctaagatattttttttctttcaaaaagagcttcgaaaacacttttccgacagaaacaagacacaaacctttttactcgtagaccgacgatttttttaaagatcaaagacctaagaagatttgtctcccatcttcttaaaacatatgtgagatagacacttctagcctcttctatgctaaaaaaaagagctcgcctttagtttggttaatttgagacaatggtctccacaaaaacataggtagttaggtactcacgcgccaagttactaatctcaaatcaaacataactgtatgatcgtcaacacattttacaagttaacaaaagagcattgctttagacttaaaaattgctttgagaaaaaacaaacacctaagaatttctaaaagcgatgatttcaactctctttaaaatgtatatatatgttacattttggtctttaaacttttcaagaaagtacaattcaactcctattttcaaaatgttcttacatgagcgatatgattttaccgtttaaaatgtaaaccatgataactatttctaaattccaatttttgtttcgttgatcaatctcattatcattcaatgcatacaaaacacaatacaatctgttttgtcatcttcttcaaggccgaaagacaactaaacatatgaagactgaagaggacaatctagcaaactcaaacttcactataaatttgcaattgctattggtagccatgaagtaacatttttgaggaggttaacaaaattttaacagtaaattcaataatccatcaccacgatatgtaatcatctacacaaacacagatgatgatagtcatatatgttcgtaatcacacaagcaagaaatcgcaatgtactttacaccgtcttctctgacttgaacacgggaattggttgtctaagaagtagattgtttcccacggatttaccaagtacatttaaatccttcaccctttgtatctctcttcctgttttttgttttgcaatcaacaaaaaaaaaagtaaaaatcccatcttaaattcacgcaaaaaaacacattgaatcaaccatcactatagtagtttcaagaaaaatctgcacttccataatttctttctattacaccattatactaggaaaacttcatagcaatattatctaaatacaaagcaatttttactagtaatattcgtagatttgtcaatatatagtgtcctaataaagaaaaaataacggaactataatgctctaataggtagattttttttagtataatgtcctaataagagaaaaaacaaatgtacaattatgtaataataagaaaagaaagtaatatgcttaatagacaaataaatgaaaccatgttgctatttcttgcatctagtccttatttgaataatatgtagcatattaactacttatgaattttatatataaatgataaaaaattccgtgag

SEQ ID NO: 11 - protein, 227 aa, Taraxacum officinale
MSTVKSKLSTPPTDHFSGNHQLLLITATAKTYIYNFHHNSVPYSHFNPKKATQTYTAMADNGNTGRQKDDDGGHDGGRPNPTTPPSPSRTPRRPRRTTSPPKHSPGASSSTMPAPPTPPAPTGITGASSSSLGTNIISFTPPKTKRTKSVICPICKKDMCHEKALCGHIRWHTQEERLAASIAIARALSSNVVVSGNGDEDEGPSKKYKLPDLNKSPPPEEEDEDAA

SEQ ID NO: 12 - DNA, 3217 bp, Taraxacum officinale
gttgaaaggaaggatcggcagagaacaagattgagagagttttatcgaagagaagagatggttgtgttgattgacggcagagttcagcaatggcagatatgcgtgtttaaagcgcagtttgtggacagtttgtccaaaggccttcaagtgggagattgttgagatgaagacccttttgaccaagtacttaaatgtgattggttgcatgggaaagagctttacacttcattggttgaaaatgaagcatgttatttaatagtacgtctccacttgttctttatgaaggatgaagacagccaccaagtctttatgaatgtttcaaggagaagacaatgattagatgctgcctgctcccttacttcttcactataaataggtgcttaaggcacctctcttcttacaccaaacaacacacaagtcacagattacaaagattgagagagttttcattttctctctagagtttgttttgtcaaaagttttgtgtgcagattaattagttgagagacactcctaattaatataaccacttacacttgtcattgattagcattctagtgtcagagagttctgagtgattgtaacctttgtgtgatttatatataaatctctctcttgttcgccagtggacgtagctgattatcctgatcagtgaaccacttaaaatatcgtgttgatattatttatctgcagattattttaccagatagttttattccgcaccgcaaagcataccgtccattatttgtcagacgtacctactctcagctcatcccagcaggagttagacctgcaggcagccagccgtatttcacaacagtgaattatgcaacaaatggaaacacacaattatcgtagtacacatgtaattaacagggatcaatttccatttattgttccatcaattgccaaatttaaaagataacaaatgcatcctttttgaatggaaaagtatgatacataaaaccagagagaggtatacatttagtccagaaacggtggaaattactttacctagagttgcgtgaatcaaatggaaaatcagaggaagagaccctaaatcgcaatccatcttctattccctatagaatcaaatggaaatttcttacttccgacctttttcatttccacaagtttgtaatcgataagatcataaaagaaaaccgatttacattagcaaatctttaaggcagtttgtagttctcaagtatgcgttacgtatgaaattctagacagaaaagggacgacagggaaaataatggtggaataaataaacaatcaagaatcaaaagataataatccttaaaactaacaaaaattctatcaataagccctacaaatgagaaatatgacggtggagactcacctgattgatgcgagaaaaggaaagccgccgacatttgtgcttcttcccgcgttaagaaaaatcgcagatgacattacgattctttttctacagaggaataggggggttgcgactttgcgtgatttgttgttaatgagtgatgcgtcgtccgttttgatcggtagcccatggagactgatttatttgacagatagatgtcgttctcgttgatgatgcaggtgtaattggcggcgttggtgaagcgaagatggcatagggaatggatttgcagaggagaaaaggagtttcacaaatatggattattgaattgtcgtttcagagtcgcgatggttagtaatcgatggatttgggtttgcacctgttcaaggggtggtgacgtgtcttactgtaagaaagagtcgcgatggttagtaatcgacgatggatttgggtttgcaacatcgttgacggaaacgacgcggcgatggagagaggcggtagagggaatgaaacgggagatttaccttcaaccgcaaacatgtcttcgttcctcgaaaatcatatgcgccctttatgaaaattccttcacgcgccgtacacgcgctcagaaaaagcatagggttttcttatataataatgatataacttaattttaaataacatcgtactatacgtttcaaaataaattagagttttaataaaattacggaatgattaaacctattacaaacaaatataagtgtcggtaatataaaatttgtaacaacgaaaacatttcaaaaaatctgaatgcggaaatccataacatgagaatacgattcgtttgcagtcacgccggtttcttcaccttctgagggtcctgaaaaaggttaataaacaatgggtaagctaaagtttagggatcatacacattacttgtctattatatgagttagatacattcacatatcacatacatacaagcatacacatattacaaaaaatcctaacacatatggttggtaccaactactatttagtggaccaggttattcttgggggtcaattgtggttaaccataaatgtttggagtttaggttctctttgggggccgaccataaacacccatatcatatctctgtctaggttctctttggggactgactatagacaactttgctcagattctccttggaggataaccatcctcgacgagattaacaacggaccaccactgttttcagcttgtaacctgcaacatcaatagttgcgatcttcggcgacaatgatgaaccaacatcaacacaacagtagtgccggtatgttagtcgttggataatagaggcatcatctgacccttttagcggtgacttttgttgcctatgaaggtcttatcagttatatatcgattaggcttggttctaggttataagaaaaaatgattctctatccggtatgacatagagacaaaccggtctacttttataagaaaatcatactcgtatttgaaacgaaaatcatatttgaaacaatgtcgtgttaataattaatatgtgtatgtttaaatttagaatatcctgaacttatatttagttcgcaaccctatgtaagatcgaactaattttaaaccgtaaccagacatgcatgtttgatcagtaataaaaaggctatcatcgtcgaaaatattaaaacaaaatataatttcttatttggcgccatttcatgtcaacggttaaaagcaagttatcaacgcctccaaccgatcatttctccggtaaccaccaactgctactaataaccgccaccgcaaaaacctatatatacaacttccatcacaattctgtcccctactcccatttcaatccaaaaaaggctacacagacatatacagcg

SEQ ID NO: 13 - DNA, 684 bp, Taraxacum officinale
atgtcaacggttaaaagcaagttatcaacgcctccaaccgatcatttctccggtaaccaccaactgctactaataaccgccaccgcaaaaacctatatatacaacttccatcacaattctgtcccctactcccatttcaatccaaaaaaggctacacagacatatacagcgatggcagataatggcaacaccggccgtcaaaaggatgacgacggtggccatgatgggggacgcccaaatccgaccaccccaccctccccttcccgcaccccgcgaagacccaggcggaccacatcgccgcctaaacattctccgggggcgtcttcaagcaccatgccagcgccgcctactccccctgcgccgacgggaatcaccggtgcgtctagttcttctctgggtactaatataatttcatttactccacccaaaaccaaaagaacgaagtcggtgatttgtccgatctgtaagaaagatatgtgccatgagaaggcgctgtgtggccacatccggtggcatactcaggaagaaagattggcggccagcattgctatagcaagagcgctatcttctaacgttgttgtttctggcaatggcgatgaagatgaaggtccatctaaaaagtataaactcccggacctgaacaagtcaccaccgccggaggaggaggacgaggacgctgcctga

SEQ ID NO: 14 - DNA, 3925 bp, Taraxacum officinale
ttttcttgcgggagaggcatgtatgtgtggcctttttgcaataatactatgcatgggagatgcataaagtttatctttattttgttttagtttaataatgtgtggcaaactttatatcatttatgatttcggccatgtactgctacatcgttctaggatggttattccctagaatttgcttttatatattttttttcttcgttaattcttctcttcctttaacgttatttcatgtgaatttgcgatatcatgatattgcaggaacgtaacttagaactcaacgataaaaagtgtgttgagatcgtggaagttgatcgtttatcttgaattcacaagataagcaacatgagatagaataattaattactgcggagatttctttttagattcatcttattcgttgtaaattgcaatgcaaatttttaagtagaataacgaaagcgtgttgttatttacggttaaatgcaaaaaatcaataacatatctccataattgatttgtttagtatgcattctcatgtgttctattgcaaaattctattttgaaattcaattaaagcaatgcattttagatataaagcaattccatcacttaattatattgggtagatttttaccaaatcactgtcctaaaagaaaaataaaaaatgaaagttcaatgccatagtgatcccgtcaattatgtgatcaatttttaccagagcaaaaccccaaaagttaaagagtataattgcagaacgacaacaatctgaggatgatactaactccggtataaaccgaatgcttttgtggggatttcactaatcaactatttggctctcatcacaaggccgtgttaatggattactattagttataaggccatcccaagttgtttggctatattattaccttttaaaaaccaaaatgtgtaaactgtaaaattacacttttggttcttgtattttctaatttttagaattatgtatttggttcttgtagtttttttatttgtatgaatcatattagttttggtccacgataacttttattttctataattacacttttggtcattgtagttttaatttgcaagaaaaaaataatctatctttttagtccatatatatagtttgcaatattctatattttaggttcaggttatatatcttttacaaaaatactacttatgattttttaatgaatatatcatatttcgtaaattacataaacattttatataattttatttagtcagaactataggattcgcatgataactttgctgaacttaattgggcttcatttttaacaaaggataatgcatcatgcatgacataaaaatttgtaatgtttctaatgtgcatgcactacaagaacctgacccttcatatacattaatgaatgattctcttttcttattatatcttagcaaacggtttggataagagtctgaacaatattaagaattgcactagctagctaggggagtccatgcagcaagcaaattaattagaggactttttaagaggcttgatcacttcattgtatatctaatggcactgtggatatatgattggattgtgtgtatgtatggaatttaatattactaaaaaatatacatttaatgacaaacaataacacactaatttataacttacaaaagcgtgtatcattaaagaaaatttcatgtttcttaaaatatggagaatttactacttacataattatgtgtcataaatttatgtcatcttgtgtgatatttatgacttacaaaaatgtatgtgatatttatgacttacaaaaatgtatgttatgtttatcgtcataaaatctgtaaaaaaaaatagaaacatggaggatactttcaatgtgtagaaacttgacagcataaacaggtttattaccttcatcaaaacttatcttacttcaatctcttggagatgcaggttgattttaatgctaaatgaacttttcaaatgtataaatatatatcttttgacggatttgacattgatggagatgaagatacaccagtgaattcacctttcaatgatcatactgacataccaagtactcttatcccctctccacctccacagaacgaacctcaatttcaaaaacgtaactgccgctgttctaaaaacagaaaattttcaaaaacgtaactatcatttaaatatttaatcatttttcaattggattcatgctgtaatagaagttactggactttaaacttcttgtagctgatgagattcatgaacaacagtccaaggagattttccaaatcaacaagtttctgatgagaacaaggtaaaaaaaataagtttttcatttaaacatatgattttggttttcaaaagataattaattataggaactcttctgaaaagtcccaaagtttacccgtaaaattgattttggttttttttttttaaattgaataaaaaagtccataaaattggataaattattcgatttagcccaatttacctagtatatctggtaaaaatgggttaaatcgaataatttaggcagtttgttgggcttttttgcgcaaaaacattattttgggacttaatcgttaattttccccaagtttcagtttatttacagtattggaataaaaccgaacctagtgcgctaattttgtcttttggtgtttagaattatttgggtttattggtctcggtttctaaacgagttcggtttttggtttcttgattcgatttttggcttttgggttcaacccatggattttcgatttggttttggttttgataatattttttgtgcaatcgttcaaaaaatccgagttagattgggtttaaatgttatcaaaaccaaaaccgatggtagaaccccaaaactgaacccaatcgaaaatccatgggttgaacctcaaaaacgaacccaataaattctaaaaaccaaaatccaaaattagcggataaaaattttaaaatacatatctactggtaaggtgtttttgacaatttatgtaaattatagaaaaaaatgtattaagtgtttgctatttaagataaaaatggcgttaaaaatacaattggttagaaatgaaccatttttttgttgtaaatgagtggtgcatcaacgatggtggatgccatcttttccttccaccatatacatctttgtcaaacaccatacacacaaattctcatatgtcaaagatattaattatcatgtccactgagtttgtaatataaatagatgatcaattacaacgagaaatatacactgaattctcatttctaaattgaagaatctctctctctctctctcatctctcagttttcaaagttttataagttactagtttatagtgtttctaattacttcatatttataacacgttatcagcacgagagctctaagcaagatatgcaattacttaataaagtttataaaaataaacaaatcataataaacaacatagatataaaattaattattaccatggacatgatggactactaacattttatttcttatactaacatttatttatgttaactaacatttatttatgtttctaacatttatttatgcatactaacatttatttatgttaactaacatttatttatgtttctaacatttatttatgcatactaacatttatttatgttactaacttttatttatgtatactaatgtttatttatcaaatttatttaacatttactattttttgattatattttttaattagccgatagaatttatttatttaattattctatttattaaacttatttttataaatatatatatatatataaccgttatatatataacggctatatatataacgtctatatatatataacggctatatttttacactataaataaacacatcattctccatttttataccacaacaaataatttctctcatcttccaaatcatcaagagaaaa

SEQ ID NO: 15 - DNA, 7826 bp, Taraxacum officinale
gttgaaaggaaggatcggcagagaacaagattgagagagttttatcgaagagaagagatggttgtgttgattgacggcagagttcagcaatggcagatatgcgtgtttaaagcgcagtttgtggacagtttgtccaaaggccttcaagtgggagattgttgagatgaagacccttttgaccaagtacttaaatgtgattggttgcatgggaaagagctttacacttcattggttgaaaatgaagcatgttatttaatagtacgtctccacttgttctttatgaaggatgaagacagccaccaagtctttatgaatgtttcaaggagaagacaatgattagatgctgcctgctcccttacttcttcactataaataggtgcttaaggcacctctcttcttacaccaaacaacacacaagtcacagattacaaagattgagagagttttcattttctctctagagtttgttttgtcaaaagttttgtgtgcagattaattagttgagagacactcctaattaatataaccacttacacttgtcattgattagcattctagtgtcagagagttctgagtgattgtaacctttgtgtgatttatatataaatctctctcttgttcgccagtggacgtagctgattatcctgatcagtgaaccacttaaaatatcgtgttgatattatttatctgcagattattttaccagatagttttattccgcaccgcaaagcataccgtccattatttgtcagacgtacctactctcagctcatcccagcaggagttagacctgcaggcagccagccgtatttcacaacagtgaattatgcaacaaatggaaacacacaattatcgtagtacacatgtaattaacagggatcaatttccatttattgttccatcaattgccaaatttaaaagataacaaatgcatcctttttgaatggaaaagtatgatacataaaaccagagagaggtatacatttagtccagaaacggtggaaattactttacctagagttgcgtgaatcaaatggaaaatcagaggaagagaccctaaatcgcaatccatcttctattccctatagaatcaaatggaaatttcttacttccgacctttttcatttccacaagtttgtaatcgataagatcataaaagaaaaccgatttacattagcaaatctttaaggcagtttgtagttctcaagtatgcgttacgtatgaaattctagacagaaaagggacgacagggaaaataatggtggaataaataaacaatcaagaatcaaaagataataatccttaaaactaacaaaaattctatcaataagccctacaaatgagaaatatgacggtggagactcacctgattgatgcgagaaaaggaaagccgccgacatttgtgcttcttcccgcgttaagaaaaatcgcagatgacattacgattctttttctacagaggaataggggggttgcgactttgcgtgatttgttgttaatgagtgatgcgtcgtccgttttgatcggtagcccatggagactgatttatttgacagatagatgtcgttctcgttgatgatgcaggtgtaattggcggcgttggtgaagcgaagatggcatagggaatggatttgcagaggagaaaaggagtttcacaaatatggattattgaattgtcgtttcagagtcgcgatggttagtaatcgatggatttgggtttgcacctgttcaaggggtggtgacgtgtcttactgtaagaaagagtcgcgatggttagtaatcgacgatggatttgggtttgcaacatcgttgacggaaacgacgcggcgatggagagaggcggtagagggaatgaaacgggagatttaccttcaaccgcaaacatgtcttcgttcctcgaaaatcatatgcgccctttatgaaaattccttcacgcgccgtacacgcgctcagaaaaagcatagggttttcttatataataatgatataacttaattttaaataacatcgtactatacgtttcaaaataaattagagttttaataaaattacggaatgattaaacctattacaaacaaatataagtgtcggtaatataaaatttgtaacaacgaaaacatttcaaaaaatctgaatgcggaaatccataacatgagaatacgattcgtttgcagtcacgccggtttcttcaccttctgagggtcctgaaaaaggttaataaacaatgggtaagctaaagtttagggatcatacacattacttgtctattatatgagttagatacattcacatatcacatacatacaagcatacacatattacaaaaaatcctaacacatatggttggtaccaactactatttagtggaccaggttattcttgggggtcaattgtggttaaccataaatgtttggagtttaggttctctttgggggccgaccataaacacccatatcatatctctgtctaggttctctttggggactgactatagacaactttgctcagattctccttggaggataaccatcctcgacgagattaacaacggaccaccactgttttcagcttgtaacctgcaacatcaatagttgcgatcttcggcgacaatgatgaaccaacatcaacacaacagtagtgccggtatgttagtcgttggataatagaggcatcatctgacccttttagcggtgacttttgttgcctatgaaggtcttatcagttatatatcgattaggcttggttctaggttataagaaaaaatgattctctatccggtatgacatagagacaaaccggtctacttttataagaaaatcatactcgtatttgaaacgaaaatcatatttgaaacaatgtcgtgttaataattaatatgtgtatgtttaaatttagaatatcctgaacttatatttagttcgcaaccctatgtaagatcgaactaattttaaaccgtaaccagacatgcatgtttgatcagtaataaaaaggctatcatcgtcgaaaatattaaaacaaaatataatttcttatttggcgccatttcatgtcaacggttaaaagcaagttatcaacgcctccaaccgatcatttctccggtaaccaccaactgctactaataaccgccaccgcaaaaacctatatatacaacttccatcacaattctgtcccctactcccatttcaatccaaaaaaggctacacagacatatacagcgatgtcaacggttaaaagcaagttatcaacgcctccaaccgatcatttctccggtaaccaccaactgctactaataaccgccaccgcaaaaacctatatatacaacttccatcacaattctgtcccctactcccatttcaatccaaaaaaggctacacagacatatacagcgatggcagataatggcaacaccggccgtcaaaaggatgacgacggtggccatgatgggggacgcccaaatccgaccaccccaccctccccttcccgcaccccgcgaagacccaggcggaccacatcgccgcctaaacattctccgggggcgtcttcaagcaccatgccagcgccgcctactccccctgcgccgacgggaatcaccggtgcgtctagttcttctctgggtactaatataatttcatttactccacccaaaaccaaaagaacgaagtcggtgatttgtccgatctgtaagaaagatatgtgccatgagaaggcgctgtgtggccacatccggtggcatactcaggaagaaagattggcggccagcattgctatagcaagagcgctatcttctaacgttgttgtttctggcaatggcgatgaagatgaaggtccatctaaaaagtataaactcccggacctgaacaagtcaccaccgccggaggaggaggacgaggacgctgcctgattttcttgcgggagaggcatgtatgtgtggcctttttgcaataatactatgcatgggagatgcataaagtttatctttattttgttttagtttaataatgtgtggcaaactttatatcatttatgatttcggccatgtactgctacatcgttctaggatggttattccctagaatttgcttttatatattttttttcttcgttaattcttctcttcctttaacgttatttcatgtgaatttgcgatatcatgatattgcaggaacgtaacttagaactcaacgataaaaagtgtgttgagatcgtggaagttgatcgtttatcttgaattcacaagataagcaacatgagatagaataattaattactgcggagatttctttttagattcatcttattcgttgtaaattgcaatgcaaatttttaagtagaataacgaaagcgtgttgttatttacggttaaatgcaaaaaatcaataacatatctccataattgatttgtttagtatgcattctcatgtgttctattgcaaaattctattttgaaattcaattaaagcaatgcattttagatataaagcaattccatcacttaattatattgggtagatttttaccaaatcactgtcctaaaagaaaaataaaaaatgaaagttcaatgccatagtgatcccgtcaattatgtgatcaatttttaccagagcaaaaccccaaaagttaaagagtataattgcagaacgacaacaatctgaggatgatactaactccggtataaaccgaatgcttttgtggggatttcactaatcaactatttggctctcatcacaaggccgtgttaatggattactattagttataaggccatcccaagttgtttggctatattattaccttttaaaaaccaaaatgtgtaaactgtaaaattacacttttggttcttgtattttctaatttttagaattatgtatttggttcttgtagtttttttatttgtatgaatcatattagttttggtccacgataacttttattttctataattacacttttggtcattgtagttttaatttgcaagaaaaaaataatctatctttttagtccatatatatagtttgcaatattctatattttaggttcaggttatatatcttttacaaaaatactacttatgattttttaatgaatatatcatatttcgtaaattacataaacattttatataattttatttagtcagaactataggattcgcatgataactttgctgaacttaattgggcttcatttttaacaaaggataatgcatcatgcatgacataaaaatttgtaatgtttctaatgtgcatgcactacaagaacctgacccttcatatacattaatgaatgattctcttttcttattatatcttagcaaacggtttggataagagtctgaacaatattaagaattgcactagctagctaggggagtccatgcagcaagcaaattaattagaggactttttaagaggcttgatcacttcattgtatatctaatggcactgtggatatatgattggattgtgtgtatgtatggaatttaatattactaaaaaatatacatttaatgacaaacaataacacactaatttataacttacaaaagcgtgtatcattaaagaaaatttcatgtttcttaaaatatggagaatttactacttacataattatgtgtcataaatttatgtcatcttgtgtgatatttatgacttacaaaaatgtatgtgatatttatgacttacaaaaatgtatgttatgtttatcgtcataaaatctgtaaaaaaaaatagaaacatggaggatactttcaatgtgtagaaacttgacagcataaacaggtttattaccttcatcaaaacttatcttacttcaatctcttggagatgcaggttgattttaatgctaaatgaacttttcaaatgtataaatatatatcttttgacggatttgacattgatggagatgaagatacaccagtgaattcacctttcaatgatcatactgacataccaagtactcttatcccctctccacctccacagaacgaacctcaatttcaaaaacgtaactgccgctgttctaaaaacagaaaattttcaaaaacgtaactatcatttaaatatttaatcatttttcaattggattcatgctgtaatagaagttactggactttaaacttcttgtagctgatgagattcatgaacaacagtccaaggagattttccaaatcaacaagtttctgatgagaacaaggtaaaaaaaataagtttttcatttaaacatatgattttggttttcaaaagataattaattataggaactcttctgaaaagtcccaaagtttacccgtaaaattgattttggttttttttttttaaattgaataaaaaagtccataaaattggataaattattcgatttagcccaatttacctagtatatctggtaaaaatgggttaaatcgaataatttaggcagtttgttgggcttttttgcgcaaaaacattattttgggacttaatcgttaattttccccaagtttcagtttatttacagtattggaataaaaccgaacctagtgcgctaattttgtcttttggtgtttagaattatttgggtttattggtctcggtttctaaacgagttcggtttttggtttcttgattcgatttttggcttttgggttcaacccatggattttcgatttggttttggttttgataatattttttgtgcaatcgttcaaaaaatccgagttagattgggtttaaatgttatcaaaaccaaaaccgatggtagaaccccaaaactgaacccaatcgaaaatccatgggttgaacctcaaaaacgaacccaataaattctaaaaaccaaaatccaaaattagcggataaaaattttaaaatacatatctactggtaaggtgtttttgacaatttatgtaaattatagaaaaaaatgtattaagtgtttgctatttaagataaaaatggcgttaaaaatacaattggttagaaatgaaccatttttttgttgtaaatgagtggtgcatcaacgatggtggatgccatcttttccttccaccatatacatctttgtcaaacaccatacacacaaattctcatatgtcaaagatattaattatcatgtccactgagtttgtaatataaatagatgatcaattacaacgagaaatatacactgaattctcatttctaaattgaagaatctctctctctctctctcatctctcagttttcaaagttttataagttactagtttatagtgtttctaattacttcatatttataacacgttatcagcacgagagctctaagcaagatatgcaattacttaataaagtttataaaaataaacaaatcataataaacaacatagatataaaattaattattaccatggacatgatggactactaacattttatttcttatactaacatttatttatgttaactaacatttatttatgtttctaacatttatttatgcatactaacatttatttatgttaactaacatttatttatgtttctaacatttatttatgcatactaacatttatttatgttactaacttttatttatgtatactaatgtttatttatcaaatttatttaacatttactattttttgattatattttttaattagccgatagaatttatttatttaattattctatttattaaacttatttttataaatatatatatatatataaccgttatatatataacggctatatatataacgtctatatatatataacggctatatttttacactataaataaacacatcattctccatttttataccacaacaaataatttctctcatcttccaaatcatcaagagaaaa

SEQ ID NO: 16 - DNA, 972 bp, Artificial Sequence
  parsley ubiquitin promoter sequence
aaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagc

SEQ ID NO: 17 - DNA, 4140 bp, Artificial Sequence
  Cas9
atggataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattcaagggctgatcctaagaagaagaggaaggtttga

SEQ ID NO: 18 - DNA, 80 bp, Artificial Sequence
  tomato U6 promoter
ggagtgatcaaaagtcccacatcgatcaggtgatatatagcagcttagtttatataatgatagagtcgacatagcgattg

SEQ ID NO: 19 - DNA, 20 bp, Artificial Sequence
  target specific sequence of RNA-1
catccggtggcatacacagg

SEQ ID NO: 20 - DNA, 20 bp, Artificial Sequence
  target specific sequence of RNA-2
ggagtagttgggttttggcg

SEQ ID NO: 21 - protein, 89 aa, Helianthus annuus
MSSPSKNKSKSSICPVCKRDLCHEKALNGHIRWHTQQEREAAGIGNAKAVASASVVIRDANVRRPETSKPVKLPDLNKSPPREEDKDAA

SEQ ID NO: 22 - protein, 157 aa, Lactuca sativa
MADDGNTARQQADAGGHGNARPNSSTPPSSPSQPPRRPRRAGATTPSKLSQAASSSTNLPPPPTPTPTPPTPSADGILLGTARRPVICPICKKDMYHEKALCGHIRWHTQEERLAASRDIARALSANVVSGQRGDGEQGPSKRFKLPDLNEPPPSED

SEQ ID NO: 23 - DNA, 36 bp, Taraxacum officinale
tgtggccacatccggtggcatacacaggaggaaaga

SEQ ID NO: 24 - DNA, 37 bp, Artificial Sequence
  pKG10821-1
tgtggccacatccggtggcatacaccaggaggaaaga

SEQ ID NO: 25 - DNA, 37 bp, Artificial Sequence
  pKG10821-4
tgtggccacatccggtggcatacacaaggaggaaaga

SEQ ID NO: 26 - DNA, 35 bp, Artificial Sequence
  pKG10821-5
tgtggccacatccggtggcatacaaggaggaaaga

SEQ ID NO: 27 - DNA, 33 bp, Artificial Sequence
  pKG10821-6
tgtggccacatccggtggcataaggaggaaaga

SEQ ID NO: 28 - protein, 12 aa, Artificial Sequence
  pKG10821-1
CGHIRWHTPGGK

SEQ ID NO: 29 - protein, 12 aa, Artificial Sequence
  pKG108121-4
CGHIRWHTQGGK

SEQ ID NO: 30 - protein, 11 aa, Artificial Sequence
  pKG10821-5
CGHIRWHTRRK

SEQ ID NO: 31 - protein, 11 aa, Artificial Sequence
  pKG10821-6
CGHIRWHKEER

SEQ ID NO: 32 - protein, 12 aa, Taraxacum officinale
CGHIRWHTQEER

SEQ ID NO: 33 - DNA, 22 bp, Artificial Sequence
  forward primer
gaaaccgaagcaaactctacca

SEQ ID NO: 34 - DNA, 20 bp, Artificial Sequence
  reversed primer
gcgctttctacaatcttaca

SEQ ID NO: 35 - DNA, 20 bp, Artificial Sequence
  forward primer
gatcgaactaattttaaacc

SEQ ID NO: 36 - DNA, 22 bp, Artificial Sequence
  reversed primer
gatcaacttccacgatctcaac

SEQ ID NO: 37 - protein, 21 aa, Artificial Sequence
  K2-2 like zinc finger domain
  misc_feature (2)..(3): Xaa can be any naturally occurring amino acid
  misc_feature (5)..(11): Xaa can be any naturally occurring amino acid
  MISC_FEATURE (12)..(12): Xaa can be K or R
  misc_feature (14)..(15): Xaa can be any naturally occurring amino acid
  misc_feature (18)..(18): Xaa can be any naturally occurring amino acid
  MISC_FEATURE (19)..(19): Xaa can be R or N
  misc_feature (20)..(20): Xaa can be any naturally occurring amino acid
CXXCXXXXXXXXAXXGHXXXH

SEQ ID NO: 38 - protein, 21 aa, Artificial Sequence
  consensus zinc finger domain
  misc_feature (2)..(3): Xaa can be any naturally occurring amino acid
  misc_feature (5)..(16): Xaa can be any naturally occurring amino acid
  misc_feature (18)..(20): Xaa can be any naturally occurring amino acid
CXXCXXXXXXXXXXXXHXXXH

SEQ ID NO: 39 - protein, 163 aa, cichorium endivia
MVDDGTTARQQVDDGGHGNPRPNPTTPPPSPSRTPRRTRRAGATTPSKLSPAVSSTTLPPPPTPPTPPADRLVTGTSSSLGTQRVRRSKSVICPICKKDMCHEKALCGHIRWHTHEERQAASSDIARALSSNFSSGHGGEEQGPSKRFKVPDLNKPPPPEEDD

SEQ ID NO: 40 - protein, 161 aa, Hieracium aurantiacum
MVDDGTAARQQVDDGGHGERRPTPTTPPPSPSRTPRRSHRPGTTPPSKLSPAASSTALPPPPTPPTPTVVAGSSSSLTTPRIKRSKSVICPICKKDMCHEKALCGHIRWHTQEERLAASGDIARALSANFVSGGNGDEEQGSSKRFKVPDLNKPPPPEEDD

SEQ ID NO: 41 - protein, 166 aa, Senecio cambrensis
MVNSGDASRSDDNPNTPNRRAGKAHAAPSTSPSSPPPPIGRLSATTPLSGSKRPHSPTKKSNFICPICGKDLYHSKALNGHIRWHSQMERELKKKALRDIRERDVAVVAAADAVQVAPVPATVPVPVPTIVRTRLFHEQPQPQPQEEVPYNYAYLLPDLNASADEE

SEQ ID NO: 42 - protein, 203 aa, Hevea brasiliensis
MVNSSPSSSSSSSSPTPPPEVQVGPGGSSVGGESEEGCSSRKKKAGSNDQQGDEEGVQKRLKRGEMDSPISEPVCCICNKKFGSWKGVFGHMRAHPEREWRGAFPPPKEKAGAININHQQILQQQLAPTLLSLGREALARMTNHHGSGAANAAAPSRRAGDTELNREQQELAGPSTIIHPSLRGFDLNLPPPPEEANEDDRNN

SEQ ID NO: 43 - protein, 230 aa, Cucurbita moschata
MEKNANTNVPSETSSDQRHGGERSPTGPTSPPPTHNRDNNPNPTTEHSSYSDTLRVPPAAETSRRDALLDVGTSYQNIVGSSEGEPGKKRGRGDGEEEEEQQQPPQQQVKAAKKKGELTEVPKGEPRCATCNKVFKSWKALFGHLRSHPERTYRGALPPPTAAELDIRRCQQQLASTLLTVAQEVAASRRGLDIDLNQPSTADDGDSPEKTGGVGFDLNADPPPESDDED

SEQ ID NO: 44 - protein, 242 aa, Eutrema salsugineum
MVTEEEINEISEDSGYLSSGEEGEIRELVCALPALNVTERLNMEEAGLHNAVVAAEFVMVASEEAPVSKENMQTVDAIDDDAKNTALGRRPRIDELDDAAGASGSTAIGSGGEDEPVKKAKKKGSSQLHAPPQGPPQCNICGRNFTSWKAVFGHLRAHKDRGYQGFLPPPTFNATEEGFSAVVLASDSSRGGVLGFGSSGIDLNLDPMEEEEEAVSGFIPKFDLNRSPPQEGQEEEEEDKAK

SEQ ID NO: 45 - protein, 244 aa, Arachis ipaensis
MEKNHSTSASTTSLPSLSLSTPVTEPPSSPLVDQPNLSGEEEKLNILKTPEKILTDLTTNNDANGGSALPPSSPPPSPPQMMVSGAIATAVGGGAVVLGGNNIIGPIKRRKASEIMDPPSGTPTCPICSKTFSSWKGAFGHMRKHPDRQYRGFFKPPTFAPSSSKAATNASGNRHEAAGEEGSSAASGEVGEGGASPAVRNLLFDLNHPVEGVKDEEEGKIILGFDLNEPAVEEEEEEKEDKPF

SEQ ID NO: 46 - protein, 202 aa, Cajanus cajan
MQEQNPSENSPSNDDAPNPQGGEGEIMKTPWQVLEELCGGSSTPVAVAPVSEGVLQPKKRKTSEVRDPPRGKPICPVCNKEFTSWKAAFGHMRAHPNRDYRGFYKPPAFPSSSSSKDQPPPSANNNKGGGAKKTNAASDGTDEVDSGGGKEVTPSPNQLFGFDLNEPVEGLGSSHAVEEGVQEEKDLGFDLNEMPPAEDHEG

SEQ ID NO: 47 - protein, 265 aa, Brassica rapa
MTNPEKMKVDGDTEKEKEDSSDEQWSDEESAMREIVLGLPALSISSATFGVSIAVDEEEEARLNEQAVVAAELVIAAAEEAVMKEKSDGKKKKVRRQRKTMKLNNDDEAGGSSKGGEAAKKPRKKTSEFTNLPRGPPVCNVCGRAFHSWKAVFGHLRSHKDRSYRGFLPPPRFSAPIRGVMIPGPNSAFVRVVARGGSSGVVVASGGGGGGGASGGEGGRGVGIVMDVDRVEKEKQEVTESGTAAKFDLNKSPPKDDDEEEDKTK

SEQ ID NO: 48 - protein, 237 aa, Lagenaria siceraria
MEKNTNANANDNTVSETSPDQRHGGERSPMASSPPPSARNDDVNNPTPNVVIELSSSVVVAAPESTTTGDVRRRQTVDATLLDVGTSSHDHVGGSSDIEQGKKRGRGDGGEQQQQVKAAKKKGELTEVPKGEPRCATCNKVFKSWKALFGHLRSHPERTYRGALPPPTAAELDIRRCQQQLASTLLTVAQQVATSRRGLNIDLNQPSAADDGDSPEKTGSVGFDLNVEPPPESDDEK

SEQ ID NO: 49 - protein, 243 aa, Arachis ipaensis
MDKNASSSSSSSSSSSSSPSTSTTEQPPPPSPQDVPVEEPPVILKTPEEVLAELTESGESPPPPPPSAAEEGGIGGSAMAIAVGVGGEGVVLGEAGSGGSGAVGASRKRKASEVKDPPTGTPTCPVCNKTFASWKGAFGHMRKHPDRQYRGFFKPPTFTSSVAAAATSGATTQGFVEGGSSAASGEGGSVGAAGQVGEGGSFDLNQPVMERGGAGDGDEGRNGDEGKDLGFDLNKPADEEDKA

SEQ ID NO: 50 - protein, 271 aa, Prunus persica
MANQQNTTTNNTNRLASSAEGQQQPGNDNVNVNVTGVAATGTPSFPNPPPPSTVQLNVARGSSVSPPGLNRGPSGRGSSNPRRVSPSGRKSPPGGRGSPSSGRGSSSFASSPGAGQITQIGGRNFTPLSVEITIGGGSGTAGGSMIPGSSSGSPPRAKRKAEITAPEGVSPVCSVCKRSNFSSWKALFGHMRCHPERQWRGCFPPPGFEEAQRALQQGERLGALRGFDLNEASDPEEENEGGGGGGFDLNMLPPDEDKDGGGSSGGAAKTG

SEQ ID NO: 51 - protein, 321 aa, Glycine max
MQNQNPSEDDSTNKAPSDDTPHPTNNDNNNPPGESLIMKTVRQVLEELGGSRDDNDGGVGDGTPVAVALEVGGGGDGSGGDGGGVGGGPPVAMEASDGGDEDNDNEHSDSIGDSVPVAVAVKVGGRGGDNSGGGATVTVSVALSDGDYGGSNGSGGSPVAVAVAVGGGSTVMESVVVGTEGVQGRKRKTPLVRDPPTGRPTCPLCQKEFQTWKGAFGHMRAHPDRDYRGFFKPPVFGSPSSTQDQPPSDGKGDDSAKKSTGEDNTAEKGSASLPVRVPMFDLNELIEEDGSSHAAEPAEDMSTGEGKGSGFDLNEMPPAED

SEQ ID NO: 52 - protein, 355 aa, Glycine max
MNHHKYSSHRHNLFNSFLTLAIFIILGFSFSATGMQNQNPSEDDSTNKAPSDDTPHPTNNDNNNPPGESLIMKTVRQVLEELGGSRDDNDGGVGDGTPVAVALEVGGGGDGSGGDGGGVGGGPPVAMEASDGGDEDNDNEHSDSIGDSVPVAVAVKVGGRGGDNSGGGATVTVSVALSDGDYGGSNGSGGSPVAVAVAVGGGSTVMESVVVGTEGVQGRKRKTPLVRDPPTGRPTCPLCQKEFQTWKGAFGHMRAHPDRDYRGFFKPPVFGSPSSTQDQPPSDGKGDDSAKKSTGEDNTAEKGSASLPVRVPMFDLNELIEEDGSSHAAEPAEDMSTGEGKGSGFDLNEMPPAED

SEQ ID NO: 53 - protein, 357 aa, Glycine max
MNHHKYSSHRHNLFNSFLTLAIFIILGFSFSATGMQNQNPSEDDSTNKAPSDDTPHPTNNDNNNPPGESLIMKTVRQVLEELGGSRDDNDGGVGDGTPVAVALEVGGGGDGSGGDGGGVGGGPPVAMEASDGGDEDNDNEHSDSIGDSVPVAVAVKVGGRGGDNSGGGATVTVSVALSDGDYGGSNGSGGSPVAVAVAVGGGSTVMESVVVGTEGVQGRKRKTPLVRDPPTGRPTCPLCQKEFQTWKGAFGHMRAHPDRDYRGFFKPPVFGSPSSTQDQPPSDGKVAGDDSAKKSTGEDNTAEKGSASLPVRVPMFDLNELIEEDGSSHAAEPAEDMSTGEGKGSGFDLNEMPPAED

SEQ ID NO: 54 - protein, 324 aa, Glycine max
MQNQNPSEDDSTKKSPSSPSDDTSHPTNNNNNPQGESLVMKTVWQVLEELGGSGDDSDGDSGFGDGAPVAVAVGGGGDDNGDGDGSVGGGATVAMEKSDDDSDNNGDNSGSNLAVAVVVGGGDGSGSSGVGATVTVSVAVSGGGGNHGGGSSGGGGGGPVAVSVAVGGGSTVVESVVVSTEGVQGRKRKTSIVRDPPTGRPTCPLCQKEFPTWKGAFGHMRAHPDRDYRGFFKPPVFGSPSSTQDQPPRDDKVAGDDSAKKSSGEVNNGEKGSASSHVRVPMFDLNEPIEEEGSSHAAEPAEDMSSEEGKGFGFDLNEMPPAED

SEQ ID NO: 55 - protein, 280 aa, Cicer arietinum
MKRFCHSFHKTEKHDDYVWPLLAHEMKKNGSRIFICDLCDKSFSSGNALGGHKASHNRSDLLQPPIKKHKLTIDSCSLSSPNDHDDVKHKHACVLCHKVFPSNKALYGHMRSHSQKDSKAIQPPLITTTSRDSKIQSNNTDDQPILPAIDLEKYFPPRSHQTKKRCSKSTIDYELINVAQILCDMSRSDPTKFTTNIDNQRNKEHVTIVKNNNNPKKLVVTFKIPKDKTFKIPKDKEEKNKEAEMEKESSHQLGSRAVAKDFDLNEIPVDLDLVADEQAP

SEQ ID NO: 56 - protein, 290 aa, Cicer arietinum
MDVDELQAAEREDRRSIIIMKRSCQSFDKPMKNDDFVWPLLANEMKKNGSRIFICDLCGKSFSSGNALGGHKASHNRSNLLQPPIKKHKLTIDSCSLSSPHDHDDVKHKHACVLCHKVFPSNKVLYGHMRSHSQKDSKAIQPPPITTTSPDSKKQSNNTHDQPILPTIDLEKYFPPRSHQTKKRRSKSIIDYELINVAQILCDMSRSDPKRLKLSTNIDNQKNKEHVTIVKNNNNEKKLVVRFKIPKDKEVKNNEGETKKESSHQLGSRGVVKDFDLNEIPVDVADEQAN

SEQ ID NO: 57 - protein, 246 aa, Cicer arietinum
MKKNGSRIFICDLCGKSFSSGNALGGHKASHNRSNLLQPPIKKHKLTIDSCSLSSPHDHDDVKHKHACVLCHKVFPSNKVLYGHMRSHSQKDSKAIQPPPITTTSPDSKKQSNNTDDQPILPTIDLEKYFPPRSHQTKKRRSKSIIDYELINVAQILCDMSRSDPKRLKLSTNIDNQKNKEHVTIVKNNNNEKKLVVRFKIPKDKEVKNNEGETKKESSHQLGSRGVVKDFDLNEIPVDVADEQAN

SEQ ID NO: 58 - protein, 6 aa, Artificial Sequence
  EAR motif
  misc_feature (4)..(5): Xaa can be any naturally occurring amino acid
DLNXXP

SEQ ID NO: 59 - protein, 5 aa, Artificial Sequence
  EAR motif
  misc_feature (4)..(4): Xaa can be any naturally occurring amino acid
DLNXP

SEQ ID NO: 60 - DNA, 1335 bp, Artificial Sequence
  Tar-MITE insert
actgctaccagggccggccagaaggctgtgcggcttgggctgtggcacaggggcagcagatttagtccatttattatggtctgctatttatttgggtccaataagatcagggcactaaaactcaaatacatcaattcaagtcggcggcaaaagaataaaaagcgccaatttttagaagacaaggagacagcacaataatcatgcttttccaatggccaattaattttagcctttgtgaggtataaatatgaatcactttttctccaaatatcattattgtaaactagtaaagagcgtaaattgttttatcgctaatcttcttctttttcaaatataattgtttgattctttgtagtaggtacgttaagttttatatcattcgtcctggaaaccttttcttatgccccttcaatttatgaatttgtgttttgtattgtcttgctattaatccaatgcaaactaattctgccgcatcgatctcacagagttggagcagcttaaaaggagttttggggcaacttaaaatgagggtagcttttttgttgttgttggtctactatttactttggtccttatcgttttagttttgtgttcattgaggatttgaatgactttgaacacaagtttataaaactaagtttttgtgattctaaatttttgatgtgtgcaaccgactaaatgaatataaaattctgatttgtgtatttctgatttctgatttaccggttattcattatgtttttctggatctcatatgaatttagtatataaaaatctcaggttcttcttactcagtctagtatattttggcattggggatcagggttaaaatcatcatttttctaagcactaacaagctttaaaaccatcaattaaaagttagttctccatacctggaattgtttattgctttagtattcatttcatagtgtcaataccttacaactgtttattgttttcaaatgtttactgttttgcactttagttgtttttagcatgtttcaactttcaaagttaattgacgatagtgtcatatacgaaatatcatcactcatacaaaattaaactggatagaattttttccgatgaaatctatttttaaatagttacgaatataataggagacttaatatgtttgtttcaaaattacacaaaaacaccatggaaatgggcattagtttgtataagcatccttttaaatataaagctttgttacattgatgccttctaaatatataatattgtaatgatttgtagtaaacaccatggaaatgggcattattaagatgtttcgcccagggcatgaaaaactatcggaccggccctgactgctac